(19) United States
(12) Patent Application Publication
(10) Pub. No.: US 2015/0267259 A1
(43) Pub. Date: Sep. 24, 2015

(54) METHOD OF PROGNOSIS AND STRATIFICATION OF OVARIAN CANCER

(71) Applicant: AGENCY FOR SCIENCE, TECHNOLOGY AND RESEARCH, Singapore (SG)

(72) Inventors: Vladimir Andreevich Kuznetsov, Singapore (SG); Zhiqun Tang, Singapore (SG); Ghim Siong Ow, Singapore (SG); Anna Vladimirovna Ivshina, Singapore (SG)

(73) Assignee: Agency for Science, Technology and Research, Singapore (SG)

(21) Appl. No.: 14/435,155

(22) PCT Filed: Oct. 11, 2013
(86) PCT No.: PCT/SG2013/000436

§ 371 Date: Apr. 10, 2015

(30) Foreign Application Priority Data
Oct. 12, 2012 (SG) 201207691-5

(51) Int. Cl. C12Q 1/68; C12N 15/113

(52) U.S. Cl. CPC C12Q 1/6886; C12N 15/1135; C12Q 2600/118; C12Q 2600/158; C12N 2310/141; C12Q 2600/178

(57) ABSTRACT

A method for the prognosis of overall survival or prediction of therapeutic outcome for a patient suffering from epithelial ovarian cancer (EOC), comprising: a. providing a metabolism response sample from the patient, b. determining the expression level of microRNA family lethal-7b (let-7b) in the sample; c. using the expression level of the let-7b to obtain the prognosis of overall survival or prediction of therapeutic outcome for the patient.

### TECHNICAL FIELD

The present disclosure relates to a method and system for prognosis of ovarian cancer, to a system and method for identifying candidate genes for use in a prognostic method, and in prognostic kits.

### BACKGROUND

Ovarian cancers are very heterogeneous diseases which lack robust diagnostic, prognostic and predictive clinical biomarkers. Conventional clinical biomarkers (stages, grades, tumor mass etc) and molecular biomarkers (CA125, KRAS, p53 etc) are not appropriate for early diagnosis, differential diagnosis, prognosis and prediction of the disease outcome for individual patients. The most common type of human ovarian cancers is human epithelial ovarian cancer (EOC). This cancer is characterized by having one of the lowest survival rates among cancers.

For the past 30 years, epithelial ovarian cancer (EOC) mortality rate has remained high and unchanged, despite considerable efforts directed toward this disease (Siegel et al, 2012). This is because EOC patients are usually diagnosed at late stage with a 5-year survival rate of only 30% (Cho et al, 2009; Karst et al, 2011; Kim et al, 2012). This high-grade epithelial ovarian cancer (HG-EOC) is normally treated as a single entity, regardless of histological or molecular subtypes. However, HG-EOC frequently exhibits very high tumor heterogeneity, genome instability and altered gene expression (Levanon et al, 2008; Shih et al, 2011), which makes the proper subtype identification and signature discovery of HG-EOC essential tasks for facilitating the development of more effective therapeutic regimens.

Previous studies of OC signature discovery have focused on the differences in the gene expression profiles in OC cancer samples or cell lines relative to normal ovarian tissue samples (Nam et al, 2008; Dahiya et al, 2008; Zhang et al, 2008; Wang et al, 2012). Given that some cell lines might not represent actual patho-biological complexity and clonal evolution of the tumors, results from cell line based studies could not be easily interpreted in the context of a paradigm shift of OC etiology and molecular classification (Vaughan et al, 2011). Recent studies suggest that the majority of HG-EOC originates from the fimbriae of the fallopian tubes, or metastasis from carcinoma of the breast, colon or other tissues (Tuma, 2010). Therefore, two HG-EOC tissue samples with similar histological subtype could display distinct biological and clinical heterogeneity in the cellular context (Cho et al, 2009; Shih et al, 2011; TOGA, 2011; Wang et al, 2005; Helfand et al, 2011; Calin et al, 2006; Chan et al, 2012), which implies a more complex HG-SOC pathobiology and complicates the search for signatures that characterize this disease.

MicroRNAs (miRNAs) are small regulatory RNA molecules processed from hairpin-shaped nucleotide precursors (pre-miRNAs) that can be incorporated into RNA-induced silencing complexes (RISC), and regulate mRNA translation and/or transcription (Lagos-Quintana et al, 2001). Most miRNAs play critical roles in vital cellular processes, as they are highly conserved across species. Human miRNAs can regulate both oncogenes and tumor suppressors, and modulate diverse cellular processes, such as development, metabolism, cell division, differentiation, and apoptosis (Calin et al, 2006; Chan et al, 2012; Valastyan et al, 2011). The oncogenic or tumor suppressive properties of specific miRNAs are complex and often ambiguous. For example, miR-138, which was identified previously as a tumor suppressor in multiple carcinomas, can function as a pro-survival oncomiR in malignant gliomas. Moreover, work has showed that overexpression of mir-138 in gliomas plays a vital role in tumor-initiating cells with self-renewal potential and is clinically significant as a prospective prognostic biomarker and chemotherapeutic target (Chan et al, 2012). Therefore, the function of a miRNA is often cell type- and context-dependent.

There remains a need to determine biomarkers for prognosis of EOC and to find improved methods for the prognosis of EOC.

### SUMMARY

The present invention proposes, in general terms, methods, systems and kits for providing a prognosis of overall survival or prediction of therapeutic outcome (for example, chemotherapeutic outcome) for a patient suffering from epithelial ovarian cancer, in which expression of let-7b and/or miRNAs with which it is associated and/or genes within which it is associated are used to provide the prognosis and/or prediction of the therapeutic outcome. In another aspect the invention proposes methods and systems for identifying miRNA and/or gene signatures for use in a prognosis or and/or prediction of the therapeutic outcome

Embodiments relate to an analytical method to identify biologically meaningful and survival-significant microRNA biomarkers and their pro-oncogenic functions and their direct and indirect gene interactors. The method may involve integrating transcriptomic and clinical information with biological knowledge to assist in selection of the most clinically relevant biomarkers.

In certain embodiments, integrative genomics and survival analysis are used to identify associations of tumor transcriptome variations and clinical heterogeneity of HG-EOC. One-dimensional Data-driven grouping (DDg) survival prediction (Motakis et al, 2009) and clustering analyses may be used to assess the prognostic ability of individual let-7 members and their gene network interactors. In certain embodiments, EOC patients may be stratified based on analysis of transcriptional co-expression patterns, biological pathways and networks of miRNAs, integrated with clinical information via consequent application of the DDg and a statistically-weighted voting grouping (SWVg) method (Kuznetsov et al, 1996; Kuznetsov et al, 2006), adapted here to multivariate survival prediction analyses assessing stratification performance of a patient cohort using the measure(s) that minimized intercomparable p-values of two or more Kaplan-Meier (K-M) curves. Following the DDg and SWVg analysis, biological pathway and network enrichment analyses, and categorical agreement analysis (Agresti, 2007) between clinical markers and the stratified sub-groups from the SWVg analysis, may be used to select the most patho-biologically reasonable and clinically significant biomarker(s) for prognoses or predictions of therapeutic outcome.

In certain embodiments, a method of prognosis and therapeutic outcome prediction of high-grade epithelial ovarian cancer (HG-EOC) based on the measurements of microRNA let-7b and/or a set of 21 let-7b associated miRNAs and/or a set of 36 let-7b associated mRNAs in a patient tumor sample is also provided. Embodiments may relate to both the methods of identification of gene or microRNA signatures, and the resulting signatures themselves.

Embodiments relate to prognostic methods and computational methods which employ let-7b and/or let-7 associated non-coding and protein-coding entities for the purpose of ovarian cancer patient stratification and disease survivability prognosis. The method may involve stratification of high-grade epithelial ovarian carcinoma patients with respect to their disease prognosis. Advantageously, the method may be carried out as an unsupervised patient stratification method, using a survival model (Cox proportional hazards model) which includes expression profile data for selection of the most statistically significant expressed genes, leading to identification of new complex biomarkers which form a statistically weighted combination of genes related to let-7b miRNA expression. Not only does the method select survival significant features, it also provides statistically-based optimal stratification of the patients regarding the risk of death or (chemo)therapeutic resistance.

The 36-protein-coding-gene and 21-non-coding-miRNA prognostic signatures of embodiments of the invention are based on the expression patterns, in patient samples, of protein-coding genes and non-coding miRNAs correlated with the let-7b expression pattern in the samples.

Particular examples are directed to:

(i) HG-EOC prognostic ability of let-7b and the 36 mRNAs encoded by protein-coding genes associated with expression pattern of let-7b;
(ii) HG-EOC prognostic ability of let-7b and the 21 coding/non-coding genes associated with expression pattern of let-7b and its associations;
(iii) let-7b as an individual or collective (i.e., together with other biomarkers including members of the 21-miRNA prognostic signature or 36-mRNA prognostic signature) biomarker of HG-EOC;
(iv) methods of patient stratification.

### BRIEF DESCRIPTION OF THE DRAWINGS

FIG. 1. illustrates analysis of let-7 family members in ovarian cancer and includes the following:

(A) Multiple sequence alignment of mature miRNA sequences of let-7 family.

(B) Heat-map of expressions of let-7 family members based on k-means clustering for TCGA dataset (top) and GSE27290 dataset (below). Greyness represents the expression values of the let-7 family members. Dark grey and light grey represent up-regulated and down-regulated miRNAs respectively.

(C) Kaplan-Meier (K-M) survival curves of three subgroups of patients (low risk 110 and 140, intermediate risk 120 and 150, high risk 130 and 160) based on SWVg analysis in TCGA (top) and GSE27290 (below) datasets, based on overall survival (OS). Stratification performance is assessed by a minimization of intercomparable p-values of K-M curves in an overall survival analysis. The log-rank P-values of the three curves are listed.

(D) K-M survival curves of two subgroups of patients with different prognosis (and risks) of death, separated by DDg analysis of the expression profiles of a possible tumor suppressor, let-7a (top), and a possible oncogene, let-7b (below), in the TCGA dataset, based on OS. The log-rank P-values of two curves are listed. In the top panel, curve 170 represents the subgroup having high expression of let-7a, and curve 175 represents the subgroup having low expression of let-7a. In the lower panel, curve 180 represents the subgroup having low expression of let-7b, and curve 185 the subgroup having high expression of let-7b.

FIG. 2 illustrates results of an embodiment of a 1-dimensional data driven grouping (1DDg) method which stratifies a patient cohort into three subgroups. The figure on the left panel indicates that the patient cohort may be represented by three subgroups which are stratified by the two expression cutoffs c₁ and c₂ associated with minimization of the log-rank p-values. The corresponding Kaplan-Meier survival curves of three groups of patients with different risks of death using cross validation, using one gene PIK3R1 (212239_at) of a 36-mRNA signature as an example, is illustrated on the right panel. In the left panel, curve 205 lying to the left of cutoff c₁ represents a first, low-risk subgroup, having survival curve 220 (right panel). Similarly, curve 210 lying between cutoffs c₁ and c₂ represents an intermediate risk group having survival curve 225, and curve 215 lying to the right of cutoff c₂ represents a high-risk group, having survival curve 230.

FIG. 3 illustrates the Kaplan-Meier overall survival curves (305: low-risk, 310: intermediate risk, 315: high-risk) of the patient subgroups, stratified via cross-validation analyses of a 36-gene signature of embodiments. The results of the cross-validation procedures showed strong agreement with the results of 1DDg-SWVg analysis, which provides a strong indication that the parameters of 1D DDg and SWVg are stable.

FIG. 4 is a summary of datasets used in examples of the invention.

FIG. 5 shows Kaplan-Meier survival curves of two subgroups of patients of TCGA dataset separated by DDg analysis of the expression profiles of individual let-7 members. In FIGS. 5A-5G, the top survival curve represents patients having high (i.e., above an expression cutoff) expression of the let-7 member, and the bottom survival curve represents patients having low (below the cutoff) expression of the let-7 member. In the FIGS. 5H and 5I, the top survival curve represents patients having low (i.e., below an expression cutoff) expression of the let-7 member, and the bottom survival curve represents patients having high (above the cutoff) expression of the let-7 member.

FIG. 6 shows survival curves generated using MIRUMIR (http://www.bioprofiling.de/GEO/MIRUMIR/mirumir.html) to assess the relationship between expression levels of let-7b and let-7c with clinical outcomes in ovarian cancer (GSE27290), breast cancer (GSE22216) and prostate cancer (GSE21036) datasets. ‘Low expression’ (L) and ‘high expression’ (H) subgroups are those where expression rank of miRNA is less or more than average expression rank across the dataset, respectively.

FIG. 7 shows correlation matrices of let-7 members in Shih's (Shih et al, 2008) and TOGA (TOGA, 2011) datasets, generated from the (A) whole dataset, (B) low-risk subgroup, (C) intermediate-risk subgroup and (D) high-risk subgroup. The number in each cell indicates the Kendall tau correlation coefficient value in cases where the p-value <0.05. An empty cell indicates that the Kendall tau correlation for that pair of miRNAs is not significant (p-value>0.05). The top left triangle in each panel shows the correlation matrix for data from the TCGA dataset, and the lower right triangle in each panel shows the correlation matrix for data from Shih's dataset.

FIG. 8 shows:

(A-B) Heatmaps of correlation values between let-7 members and 141 miRNAs for (A) TCGA and (B) Shih's dataset.

(C-D) Heatmaps of correlation values between let-7 members and 21 significant miRNAs for (C) TCGA and (D) Shih's dataset.

(E-F) Kaplan-Meier survival curves for dataset (E) TCGA and (F) GSE27290, generated via 1DDg and SWVg. In panels E and F, curves for low-risk (L), intermediate-risk (I) and high-risk (H) subgroups are shown.

Greyness in the heatmaps represents the correlation values of miRNA-mRNA probe pairs respectively. Dark grey and light grey represent positively and negatively-correlated respectively.

FIG. 9 illustrates analysis of correlated genes of let-7 family members and includes the following:

(A) Frequency distribution plots of Kendall-tau correlation coefficients across all 364 samples for each member of let-7 family, compared to the let-7 family and the entire background consisting of 2,571,080 miRNA-mRNA pairs (136 miRNAs vs 18905 mRNAs). The vertical dotted lines located at Tau=−0.122 and +0.122 specify the statistically significant FDR cut-off of 0.01.

(B) Flow-chart of extracting significant probesets for GO and pathway analysis. A Benjamini-Hochberg corrected p-value (FDR or q-value) of 0.01 was imposed and 2,971 mRNA probes that were significantly correlated with let-7b in both positive and negative direction were extracted. GO analysis was performed for both the positively correlated genes and negatively correlated genes of let-7b (DAVID Bioinformatics). Venn diagram of significant GO terms (q-value <0.05) revealed that gene functions associated with positively correlated genes and negatively correlated genes are distinct.

(C) Pathway enrichment analyses on both sets of probes were performed using Metacore™ from GeneGo Inc. A total of 162 genes (corresponding to 238 probes) were extracted from significant pathways (q-value <0.001) for further survival prediction analysis and signature selection.

(D) Survival significance of each of the 162 genes was assessed using one-dimensional data-driven grouping (DDg) method. The top-ranked survival-significant genes were further assessed via statistically weighted voting grouping (SWVg) to generate a survival gene signature. The 36-mRNA prognostic signature with involvement in DNA damage repair, cell cycle, cell adhesion, regulation of epithelial-to-mesenchymal transition and immune response, can provide strong stratification of the patients according to Kaplan-Meier survival curves for overall survival (OS) derived by SWVg via minimization of p-values in inter-comparison of Kaplan-Meier survival curves p-value=1.27E-19. Survival curves for low-risk (L), intermediate-risk (I) and high-risk (H)_subgroups stratified using the 36-mRNA signature are shown.

FIG. 10 is a heatmap showing clusters of significantly correlated mRNA probes with the 9 miRNAs of the let-7 family. Only mRNA probes that show significant correlation (FDR ≦0.01) with at least one of the 9 let-7 miRNAs are considered in this clustering analysis. Hierarchical clustering algorithm (clustering method: centroid linkage; similarity metric: Kendall-tau) was implemented. Greyness represents the correlation values of miRNA-mRNA probe pairs respectively. Dark grey and light grey represent positively and negatively-correlated respectively.

FIG. 11 shows Kaplan-Meier survival curves of Clinical indicators (FIG. 11A-FIG. 11E) and conventional biomarkers (FIG. 11F-FIG. 11I) of SOC disease. The survival curves in FIG. 11F-FIG. 11I) were obtained from the 1DDg analysis of the TCGA dataset. FIG. 11J shows the Kaplan-Meier survival curves of four gene-based clusters from TCGA data analysis in literatures (TCGA group, Nature 474:609-15, 2011). In FIG. 11A, curve 1101 represents stage I-II tumors while curve 1102 represents stage III-IV tumors; in FIG. 11B, curve 1103 represents low grades (1,2) while curve 1104 represents high grades (3, 4); in FIG. 11C, curve 1105 represents patients having residual disease with tumor size >1 mm and curve 1106 represents patients with no macroscopic disease; in FIG. 11D, curve 1107 represents patients having complete response to primary chemotherapy, curve 1108 partial response, curve 1109 progressive disease, and curve 1110 stable disease; in FIG. 11E, curve 1111 represents loco-regional recurrence and curve 1112 metastasis. In each of FIGS. 11F to 11I, H indicates the high-risk group and L indicates the low-risk group.

FIG. 12 relates to validation of the 36-mRNA prognostic signature in the TOGA dataset and shows a comparison of the log-rank p-value of our 36-mRNA prognostic signature with the log-rank p-values of randomly generated signatures having the same size. (FDR=3.01e-03).

FIG. 13 illustrates independent evaluation and function analysis of the 36-mRNA prognostic signature and includes the following:

(A)-(C) Independent evaluation of the 36-mRNA prognostic signature. The three subgroups from independent datasets were predicted using the prediction model generated by our method from The Cancer Genome Atlas (TCGA) dataset (with same gene design and weight). The survival curves in Figure A, B and C were obtained from 230 tumor samples in GSE9899, 130 samples from GSE26712, and 157 samples from GSE13876, respectively. One of 36 genes (TUBB) is absent in dataset GSE13876. So, the 35 genes were utilized to generate the SWVg stratification model. L=low-risk, I=intermediate-risk, H=high-risk.

(D) Boxplots of log 2-expression levels for representative survival prognostic signature (SPS) genes that are survival significant as selected by our voting algorithm and that are also differentially expressed between the distinct prognostic (and risk) groups, as defined by the SPS.

(E) A model of let-7b-mediated transcriptional regulation in HG-SOC prognoses chemotherapy response and overall patient survival.

FIG. 14 illustrates EMT pathways where seven EMT pathway genes are included within the 36-mRNA prognostic signature. Each of the 7 EMT genes, for example HGF and FZD1, exhibits significant oncogenic pattern in context of disease progression: an over-expression of these genes is associated with poor prognosis in TCGA SOC patients (see FIG. 15).

FIG. 15 shows survival patterns of seven EMT genes included within the 36-mRNA prognostic signature. Each of the 7 EMT genes exhibit significant oncogenic pattern in TCGA SOC patients. H=high expression, L=low expression.

### DETAILED DESCRIPTION

Bibliographic references mentioned in the present specification are for convenience listed in the form of a list of references and added at the end of the examples. The whole content of such bibliographic references is herein incorporated by reference.

The present inventors have found from computational analyses of EOC datasets that let-7b is an important member of the let-7 family exhibiting pro-oncogene characteristics and directly involved in progression of HG-EOC. Based on this, embodiments of the invention (i) identify 21 non-coding microRNAs which are significantly correlated with let-7b, (ii) identify a subset of let-7b associated genes significantly enriched for biological pathways which are critical for cancer progression and prognosis of patient survival, (iii) identify a let-7b associated 36 protein-coding gene prognostic signature from (ii) that can stratify HG-EOC patients into three survival significant clinical subgroups (low-, intermediate- and high-disease prognostic risk subgroups, significantly differentiated by the minimization of intercomparable p-values of K-M curves in the overall survival (OS) analysis, the corresponding tumors of which are considered to be distinct by virtue of the statistical significance of enrichment of the genes involved in specific biological pathways, and which differ in sensitivity to primary therapy. Embodiments also make use of the results of (i-iii) and propose the use of let-7b and/or the let-7b associated 21-miRNA prognostic signature and/or let-7b associated 36-mRNA prognostic signature in a kit pr prognostic assay for prediction of overall survival time and treatment outcome of individual HG-EOC patients in a clinical setting.

The present inventors have found that genes of the 36-mRNA prognostic signature are involved in pathways of immune response, cell-adhesion, DNA damage repair, cell cycle, and regulation of epithelial-to-mesenchymal transition which could constitute, independently or in various combinations, small-dimension survival prediction signatures of HG-EOC.

Currently, patients diagnosed with stage III-IV HG-EOC have poor prognosis where only 20-30% survive after 5 years. However, embodiments of the present invention can further stratify these patients into one of three disease prognostic risk subgroups, of which the low-risk subgroup has a relatively good 5-year survival rate of 65-72%. On the other hand, the intermediate- and high-risk subgroups have 5-year survival rates of 20-35% and 0-10% respectively. Furthermore, the high-risk subgroup is significantly correlated with the mesenchymal molecular subtype, which often exhibited stem-cell like properties of which chemo-resistance do not respond favorably to treatment, which contributes to a very poor mortality rate. The high-risk subgroup is also significantly associated with large tumor residual size or poor patient response after primary therapy. Contrary to that, the low-risk subgroup is significantly correlated with proliferative-subtype, of which the fast-dividing cancer cells could be sensitive to chemo-therapy. Embodiments use the biologically and clinically relevant 36-mRNA prognostic signature as a high-confidence prognostic tool to significantly stratify HG-EOC patients into three survival-significant, molecularly different and clinically distinct subclasses, which can improve patient risk assessment, management and counseling, as well as provide a solution for the optimization of personalized medicine strategy of treating human ovarian cancers in a clinical setting. Embodiments relate to a method of prognosis and outcome prediction of high-grade epithelial ovarian cancer (HG-EOC) based on the measurements of microRNA let-7b, the 21 let-7b associated miRNAs and the 36 let-7b associated mRNAs in the patient tumor samples.

Embodiments relate to the methods of identification and use of the resulting gene or microRNA signatures.

Embodiments may include one or more of the following features:

i) the identification of let-7b as an important master regulator and pro-oncogenic miRNA of the let-7 family in HG-EOC. This is based on a modification of data-driven grouping (DDg) analysis method predicting patient survival based on let-7b expression level in tumor cells and correlation analyses of let-7 family members' gene expression with expression levels of direct and indirect gene targets defined in the HG-EOC patient transcriptomes using microarray signals. DDg is a computational method, which classifies the patients into low and high-risk subgroups through the optimization of statistical difference between the two (or three) Kaplan-Meier survival curves generated by the optimal expression cut-off value of each gene. The cutoff value for a gene is generated based on expression data of that gene across a plurality of patient samples.
ii) the use of expression correlation analysis to identify microRNAs which are significantly associated with let-7b. In a particular example, the expression correlation analysis generates a 21-miRNA signature.
iii) the use of expression correlation and pathway enrichment analyses to identify a representative subset of let-7b-associated mRNA genes that are both significantly correlated with let-7b across all HG-EOC patients and are involved in the most statistically significantly enriched biological pathways which are critical for progression and metastasis of cancer.
iv) the use of DDg and a statistically-weighted voting grouping (SWVg) method to identify from (iii), a subset of biologically meaningful and survival significant genes that can provide clinically distinct and statistically significant stratification of HG-EOC patients into low-, intermediate- and high-risk subgroups, defined by the SWVg method, adapted to survival prediction analysis. The SWVg is a computational disease outcome prediction method that performs a goodness-of fit analysis to separate a cohort of patients into two or more subgroups belonging to distinct K-M curves. The K-M curves are constructed in a survival analysis using the multivariate Cox proportional model. The SWVg is used to obtain a consensus grouping decision from the grouping information (e.g. groups based on individual survival significant genes) generated from the DDg method. The initial patient cohort splitting performance is assessed via minimization by the SWVg via an assessment of intercomparable p-values of K-M curves in the multivariate overall survival data analysis. The log-rank p-values are used in the assessment. SWVg can be applicable to data generated from different kind of assays including but not limited to microarrays, PCR-based and sequencing-based detection systems (e.g. TaqMan, RNA-seq)

In a particular example, the combination of DDg and SWVg generates a 36-mRNA signature which provides the separation of a given patent group into the three statistically different overall survival subgroups.

Embodiments of the method may involve the analysis of gene and/or miRNA expression in tumour tissue samples, which can be obtained by biopsy. Expression analysis may also be performed using peritoneal sample tests, smear tests and blood tests. Samples used in expression analysis can be obtained from body fluids, for example blood, lympha, ascites, pleural fluid, peritoneal fluid, pericardial fluid, sputum, saliva, and urine.

Embodiments of the present invention provide the following advantages:

i) provide the stratification of large cohorts of HG-EOC patients into three distinct molecular subgroups with differential overall survival based on the expression values of the let-7b and the genes of the 36-mRNA signature.
ii) facilitate the study of each molecular subgroups defined in (i), with respect to their molecular features and tumor etiology of HG-EOC. In particular, regulation of EMT appears to be a practically important mechanism, and allows identification of biomarkers which can assist in discriminating into low-, intermediate- and high-risk subgroups.
iii) be used as a prognostic and primary (chemo)therapy outcome predictive tool in the clinics for patients diagnosed with HG-EOC based on the expression values of let-7b, let-7b associated 21-miRNA non-coding genes and let-7b associated 36-mRNA protein coding genes.

Embodiments may relate to one or more of the following:

1. A method of identifying biologically meaningful (significantly enriched with specific biological categories) and survival-significant gene signatures via integrating the sub-transcriptome of the genes correlated with the expression pattern of a given microRNA, and clinical information about patient survival with biological knowledge derived by application of pathway and/or network enrichment analysis, Data-Driven Grouping (DDg) analysis followed by Statistically-weighted voting grouping (SWVg).

2. A method of identifying therapeutic gene targets via integrating the sub-transcriptome of the genes correlated with expression pattern of a given microRNA and clinical information about patient survival with biological knowledge derived by application of pathway/network enrichment analysis and Data-Driven Grouping (DDg) analysis followed by Statistically-weighted voting grouping (SWVg).

3. A method to predict therapy outcome and classify cancer patients into low-, intermediate- and high-risk subgroups by measuring the expression levels of microRNA let-7b, a 21-miRNA prognosis signature and/or a 36-mRNA prognosis signature. Prediction of therapeutic outcome includes predicting whether a patient is likely to respond to therapeutics such as chemotherapeutic agents.

4. A 36-mRNA signature for prognosis of EOC as follows—DNMT1, CFD, CD93, MMP13, ARPC1B, CD44, PIK3R1, GNG12, CCL2, PLAUR, LAMA4, COL3A1, VCL, CAV2, FZD1, CALD1, EDNRA, TGFBR2, PDGFRA, FGFR1, HGF, POLR2D, POLR2J, CDK4, CHEK1, CCT2, CDC6, TUBB, NCAPD2, NCAPG2, POLA2, MCM2, TCP1, NCAPH, CBX3, and MIS12. In exemplary embodiments, a low-risk subgroup defined by the 36-mRNA prognosis signature has a 5-year overall survival rate of 65-72%, an intermediate-risk subgroup has a 5-year overall survival rate of 20-35%, and a high-risk subgroup has a 5-year overall survival rate of 0-10%.

5. A 21-miRNA survival signature for EOC prognosis as follows—miR-107, miR-103, miR-106b, miR-18a, miR-17-5p, miR-20b, miR-183, miR-25, miR-324-5p, miR-517c, miR-200a, miR-429, miR-200b, miR-96, miR-362, miR-127, miR-214, miR-136, miR-22, miR-320 and miR-486. In exemplary embodiments, a low-risk subgroup defined by the 21-miRNA prognosis signature has a 5-year overall survival rate of 53%, an intermediate-risk subgroup has a 5-year overall survival rate of 22%, and a high-risk subgroup has a 5-year overall survival rate of 8%.

6. A method of treating cancer in a subject by modulating the expression of protein-coding and/or non-coding genes that are positively correlated or negatively correlated with let-7b.

Results of analyses performed by the present inventors suggest that genes that are positively correlated or negatively correlated with let-7b in epithelial ovarian cancer could be involved in anti-apoptotic and apoptotic processes respectively. Furthermore, classification of the patients into the three distinct risk subgroups, followed by differential expression analysis revealed that genes up-regulated in the high-risk subgroup with respect to the low-risk subgroup are significantly enriched in negative regulation of apoptosis (FDR=0.0070) and anti-apoptosis (FDR=0.0072).

The 36-mRNA prognosis signature stratifies patients into three subgroups with different overall survival and primary therapy outcome. The mRNA signature may offer some suggestions (supported by statistical testing) whether a patient is likely to respond to primary (chemo) therapy.

Advantageously, embodiments of the presently disclosed method can perform prognostic feature selection on very high-dimensionality, noisy and mixture biomarker spaces and stratification. The prognostic feature selection method can be broadly used in prognosis of many types of diseases and medical conditions. Via survival data modeling and integration with statistically significant and biologically meaningful prognostic features, this method can be applied for analyzing any complex clinical data sets and used in disease subtypes classification, disease prognosis prediction, treatment assignment making decision, clinical trials design and clinical biomarkers discovery.

In an exemplary embodiment, a DDg-SWVg-based analysis was used to identify a subset of 36 mRNAs associated with let-7b that could stratify HG-EOC patients into three distinct disease prognosis risk subgroups where the low-risk subgroup has a 5-year overall survival rate of 65-72%. The p-values discriminating survival subgroups are 1.27E-19 (TCGA as training dataset) and 2.54E-17 (AOCS dataset, GEO accession number GSE27290, as test dataset). The 36-mRNA prognosis signature is represented by 7 genes (FZD1, CALD1, EDNRA, TGFBR2, PDGFRA, FGFR1, and HGF) involved in regulation of epithelial-to-mesenchymal transition, which suggests that the signature reflects specific molecular mechanisms related to ovarian cancer progression and to HG-EOC patient survival. The 36-mRNA signature is represented by 6 genes (PDGFRA, CDK4, CCL2, DNMT1, LAMA4 and GNG12) which were found in the published literature to be related to ovarian cancer, and 30 genes not previously associated with ovarian cancer. The 36-mRNA signature, as a composite biomarker, is able to stratify patients with HG-EOC into survival significant subgroups based on their risk of death or (chemo)therapeutic resistance. Accordingly, embodiments of the present invention provide for classification of patients already diagnosed with the disease into more discriminative survival subgroupings/stratification as compared to previously known methods. The signature can be implemented as a test/kit for survival prognosis of the HG-EOC patients.

In another exemplary embodiment, a DDg-SWVg-based analysis was used to identify 21 microRNAs which are significantly correlated with let-7b. Among the 21 microRNAs, 14 of them (miR-107, miR-103, miR-106b, miR-18a, miR-17-5p, miR-20b, miR-183, miR-25, miR-324-5p, miR-517c, miR-200a, miR-429, miR-200b, miR-96) are negatively correlated with let-7b and let-7c, while 7 of them (miR-362, miR-127, miR-214, miR-136, miR-22, miR-320, miR-486) are positively correlated. Overexpression of the 7 miRNA subset positively correlated with expression of let-7b provides relatively poor prognosis for HG-EOC, while overexpression of the 14 miRNA subset provides relatively good prognosis for the disease. Six miRNAs (miR-324-5p, miR-320, miR-136, miR-214, miR-17, and miR-18a) are survival significant (DDg p-value 0.01). Combining the 6 miRNAs into a survival signature could provide strong classification of patients according to their survival profile (p-value=6.26E-11). Furthermore, a signature comprising of all 21 miRNAs that are correlated with let-7b could provide further improvement in patient stratification (p-value=1.03E-12). The 21 miRNAs can significant stratify patients diagnosed with HG-EOC into low-, intermediate- and high-risk subgroups, where the 5-year survival rate is 8%, 22% and 53% respectively (p-value=1E-12). This result suggests that a signature comprising of 21-miRNAs or a signature comprising a subset of the 21 miRNAs could also be used as potential biomarkers of HG-EOC patient stratification.

Advantageously, generation of biologically meaningful gene signatures can be performed in an automated and unsupervised fashion.

In certain embodiments, methods of identifying candidate genes make use of a data-driven grouping (DDg) method which stratifies a patient cohort into two partitions, as described in Motakis et al (2009), US Patent Publication 20110320390 and US Patent Publication 20120004135, the entire contents of each of which are hereby incorporated by reference. In other embodiments, a generalization of the two-partition DDg method is possible, in which the DDg method can be used to partition a patient cohort into three (or possibly more than three) partitions wherever appropriate or meaningful. Briefly, DDg is a computational statistical-based method of identification of survival significant genes. This method is based on fitting a semi-parametric Cox proportional hazard regression model, which is used to fit patients' disease free survival times (t) and events (e) to a gene's expression data (y). The model estimates the optimal partition (cut-off) of a gene's expression level by maximizing the separation of the survival curves related to the high- and low-risk of the disease behavior (for two partitions) or low, intermediate and high-risk of the disease behavior (for three partitions). The method can identify single genes that exhibit a statistically significant influence on patients' survival and can divide patients into two or three distinct subgroups. In the presently described DDg analysis, an individual gene is ranked based on its ability to significantly classify patients into two or three subgroups. As a further optional step, the SWVg procedure uses the ranked list of genes from the DDg analysis to obtain a consensus grouping decision from the respective groups generated by two or more genes. The SWVg method selects statistically significant genes which were derived from a plurality of DDg models, each of which represents a way of partitioning a set of patients based on the optimal cut-off values of gene expression. Those genes are identified based on which one of the models has a high prognostic significance.

Embodiments of the present invention can be used as a prognostic tool to significantly stratify HG-EOC patients into three survival-significant molecularly different and clinically distinct subclasses can improve patient risk assessment, management and counseling, as well as provide a solution for the optimization of personalized medicine strategy of treating human ovarian cancers in a clinical setting. Currently, patients diagnosed with stage III HG-EOC have poor prognosis where only 30% survive after 5 years. Embodiments of the present invention, via the 36-mRNA (protein-coding) or 21-miRNA (non-protein coding) signature can further stratify these patients into more discriminative risk subgroups (low-risk, intermediate-risk and high-risk) which is an indication of the heterogeneous nature of this disease. In a clinical setting the present methods may be used by clinicians for patient prognosis, prediction of primary (chemo)therapy efficacy as well as the design of future personalized therapeutic intervention. Let-7b, as well as individual genes, subsets, and all genes of 36-mRNA and/or 21-miRNA prognostic signatures could be used as prognostic biomarker kits and assays.

Having now generally described the invention, the same will be more readily understood through reference to the following examples which are provided by way of illustration, and are not intended to be limiting of the present invention.

A person skilled in the art will appreciate that the present invention may be practised without undue experimentation according to the method given herein. The methods, techniques and chemicals are as described in the references given or from protocols in standard biotechnology and molecular biology text books.

### EXAMPLES

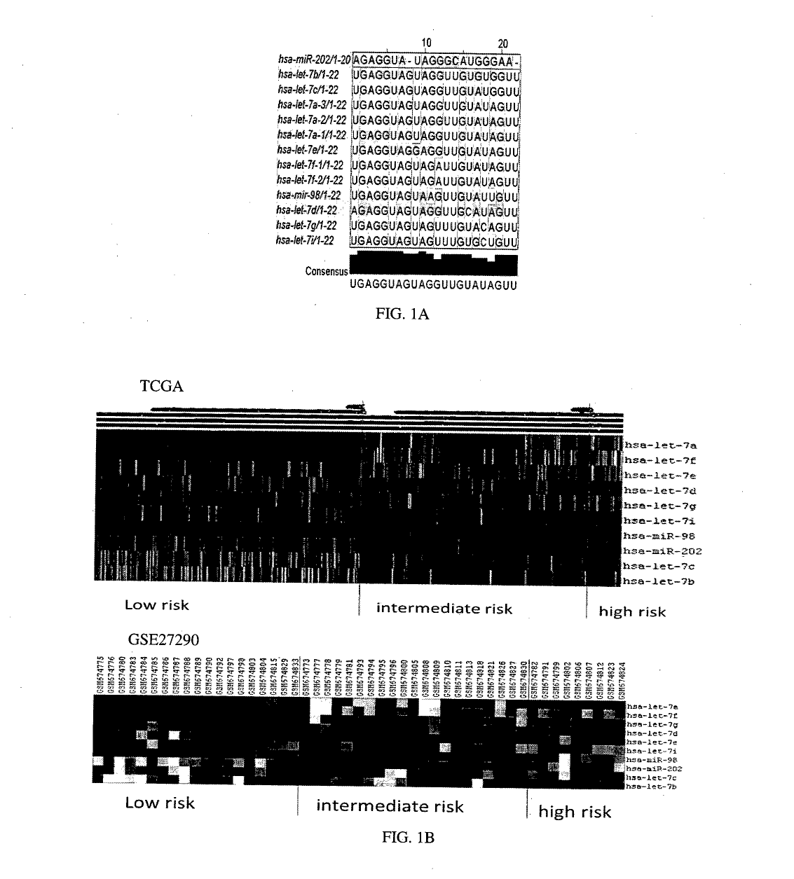

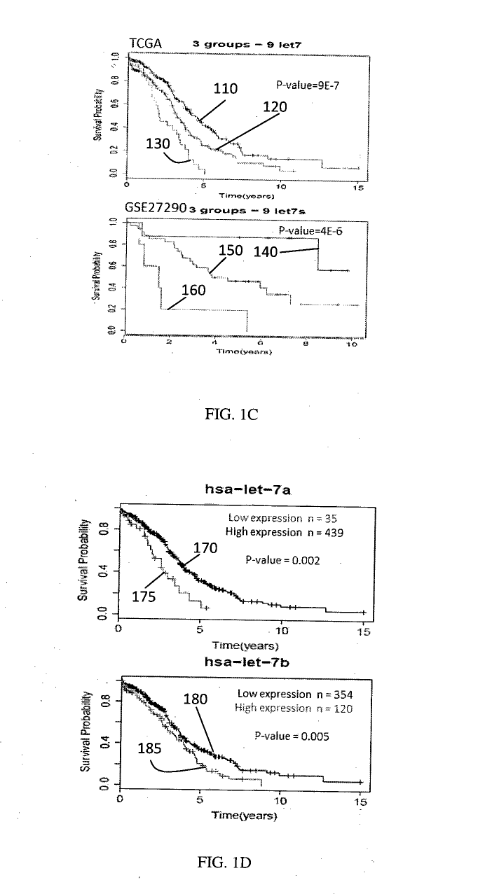

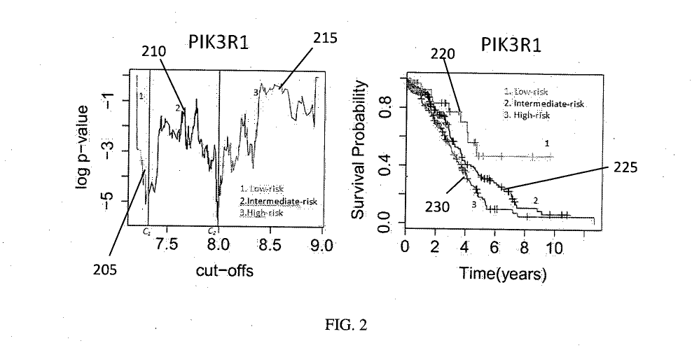

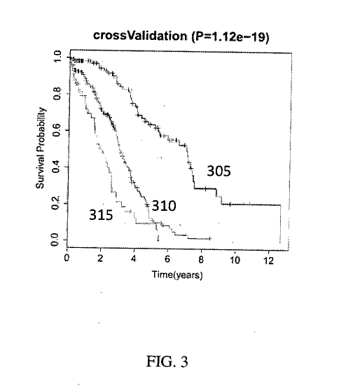

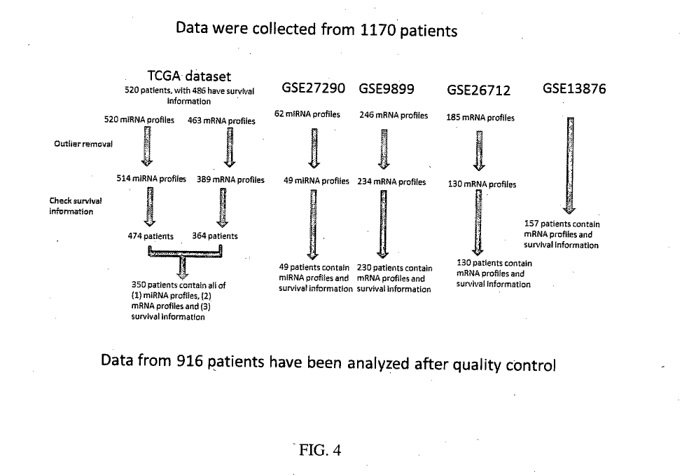

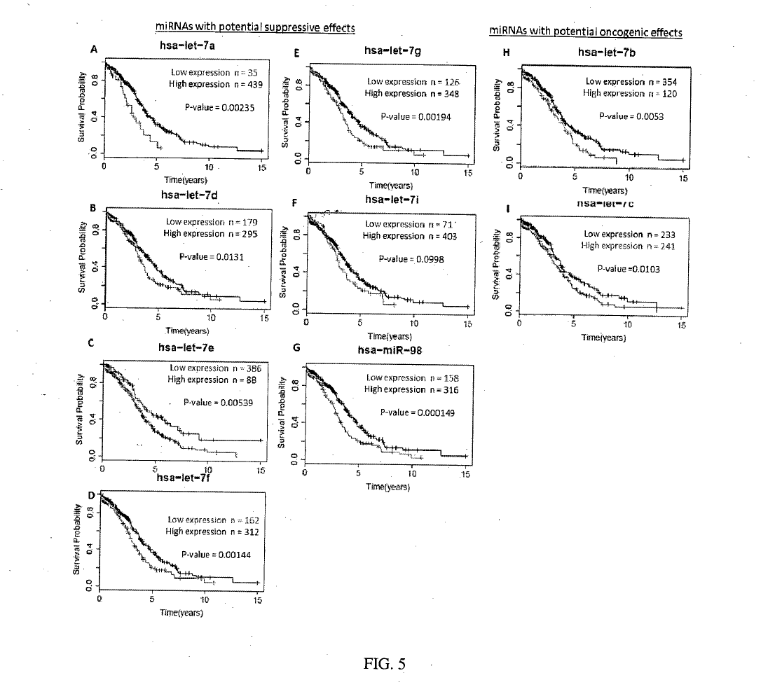

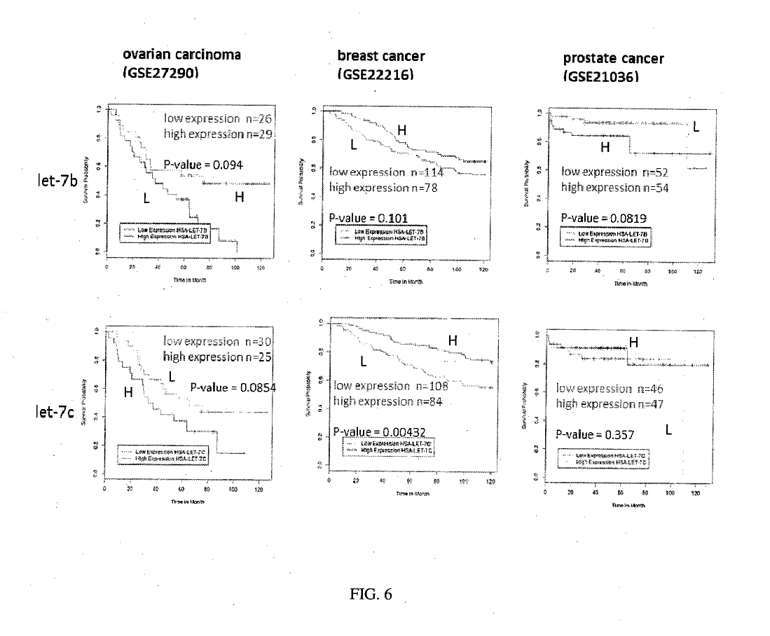

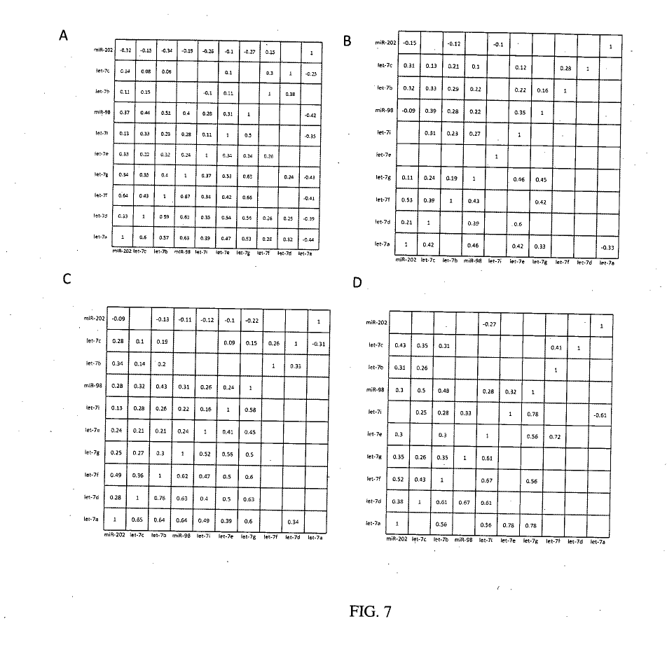

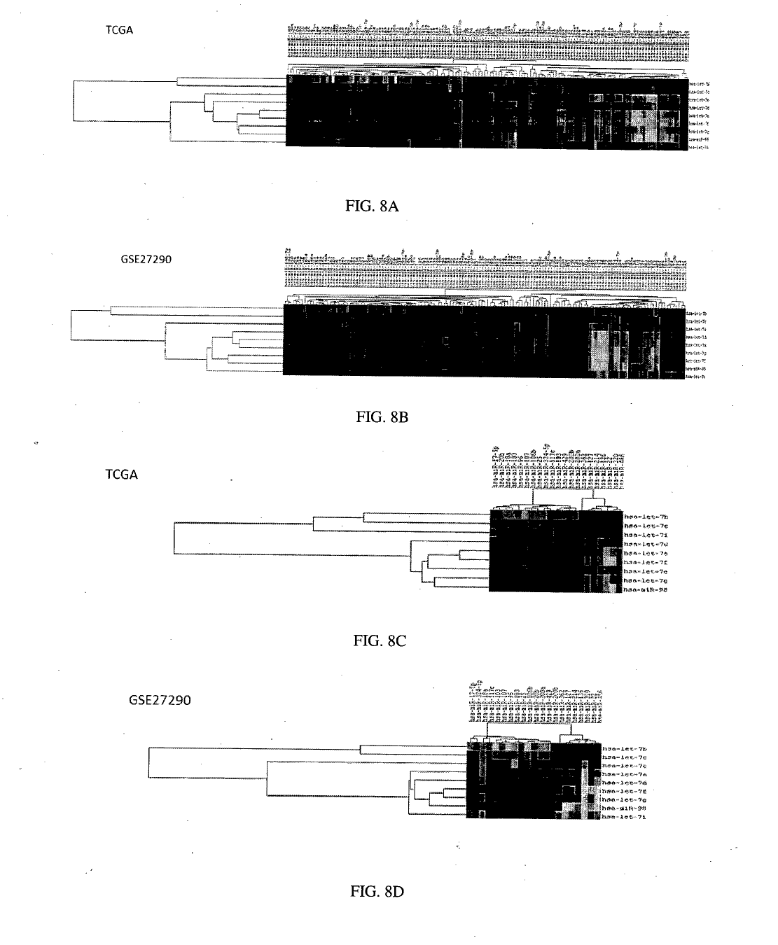

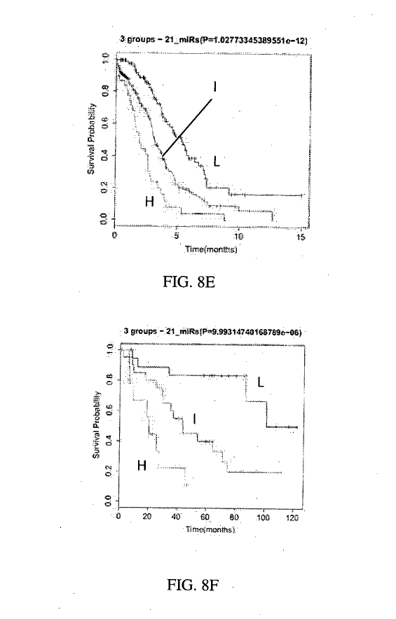

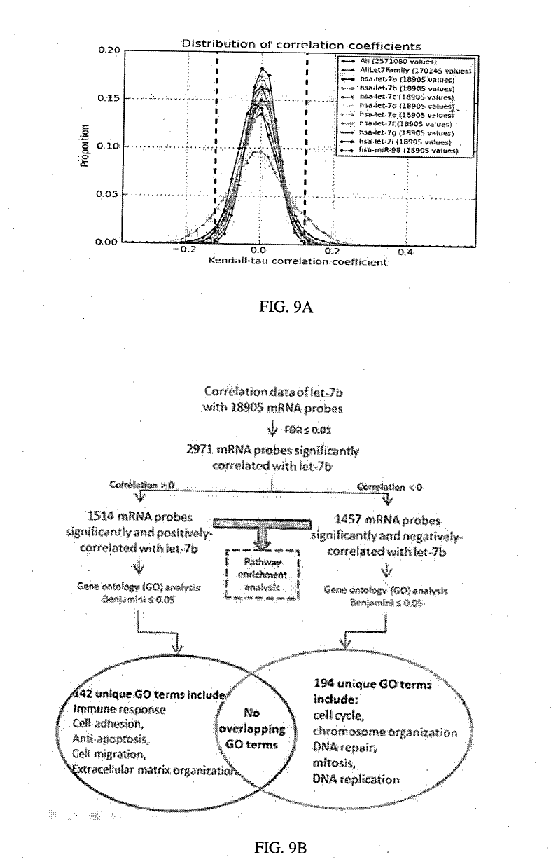

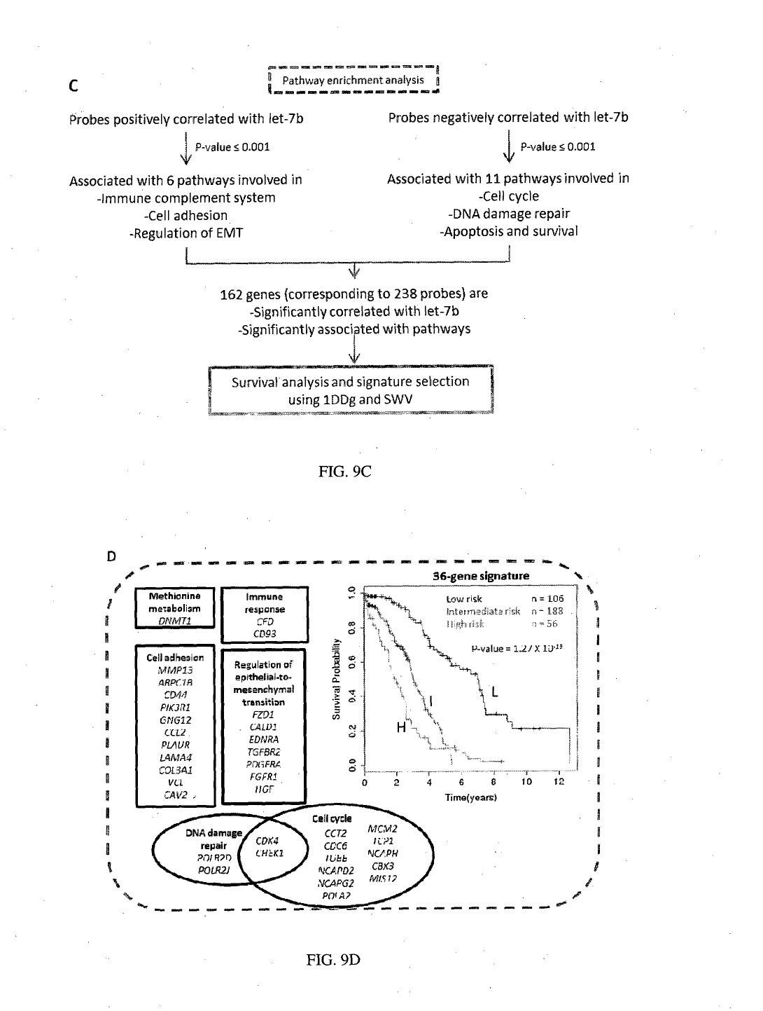

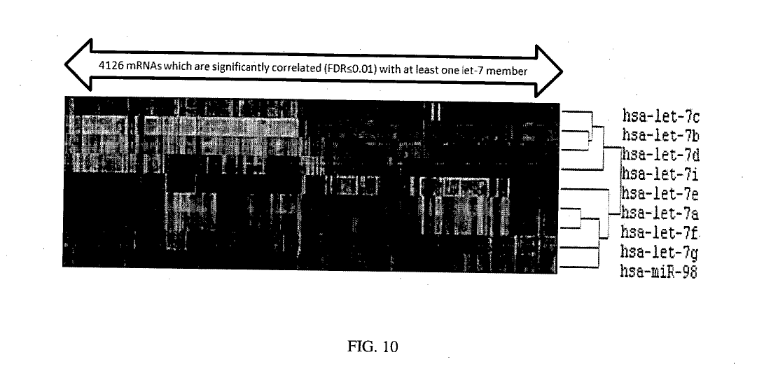

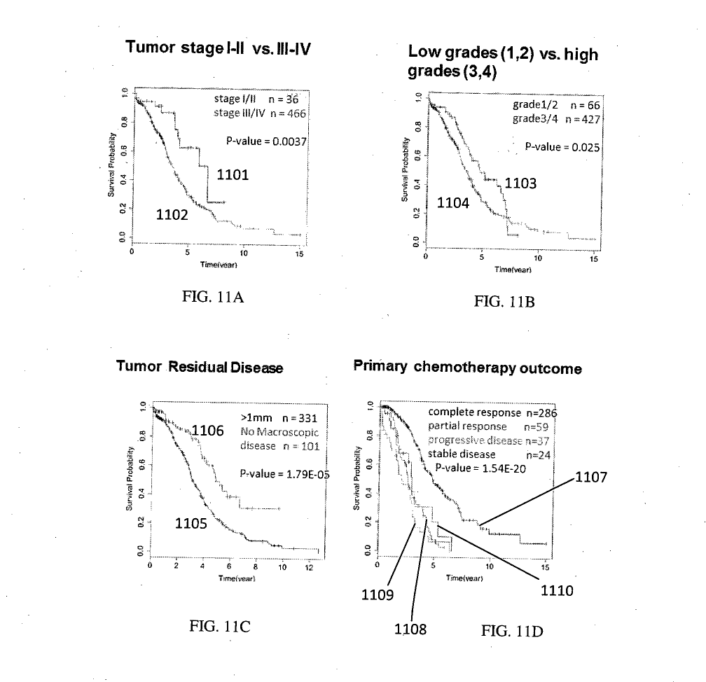

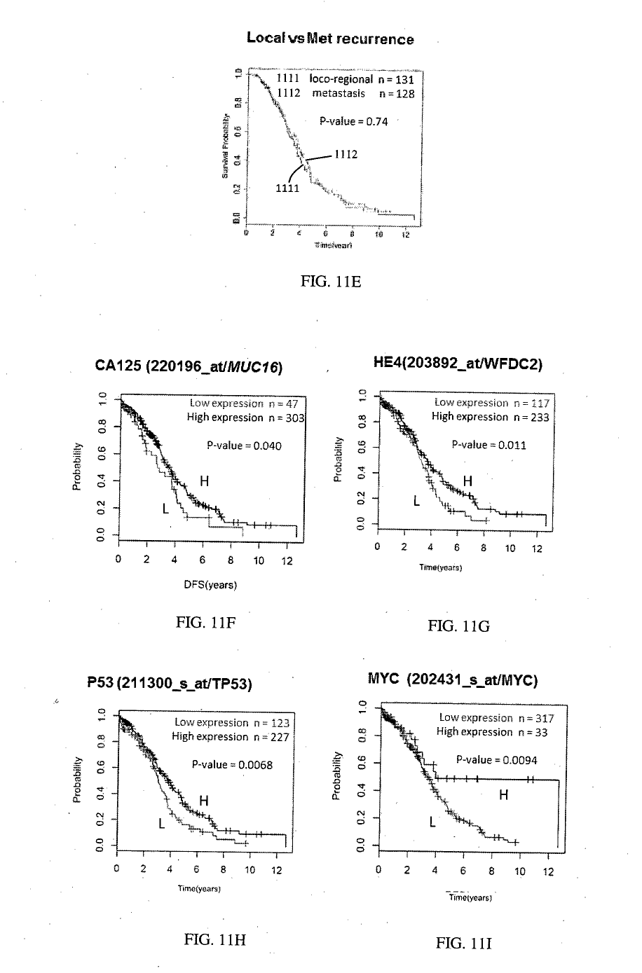

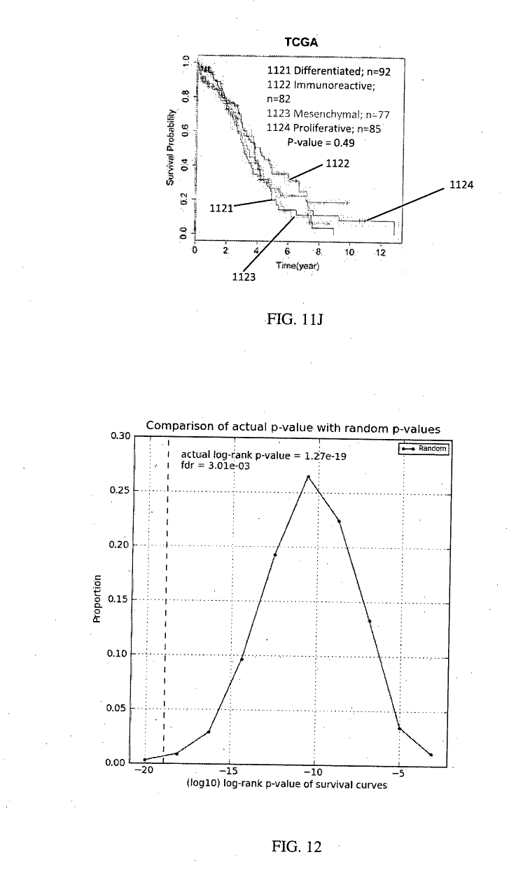

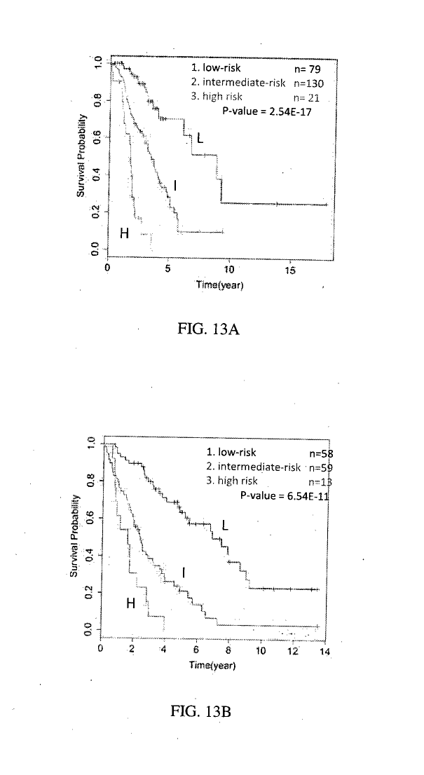

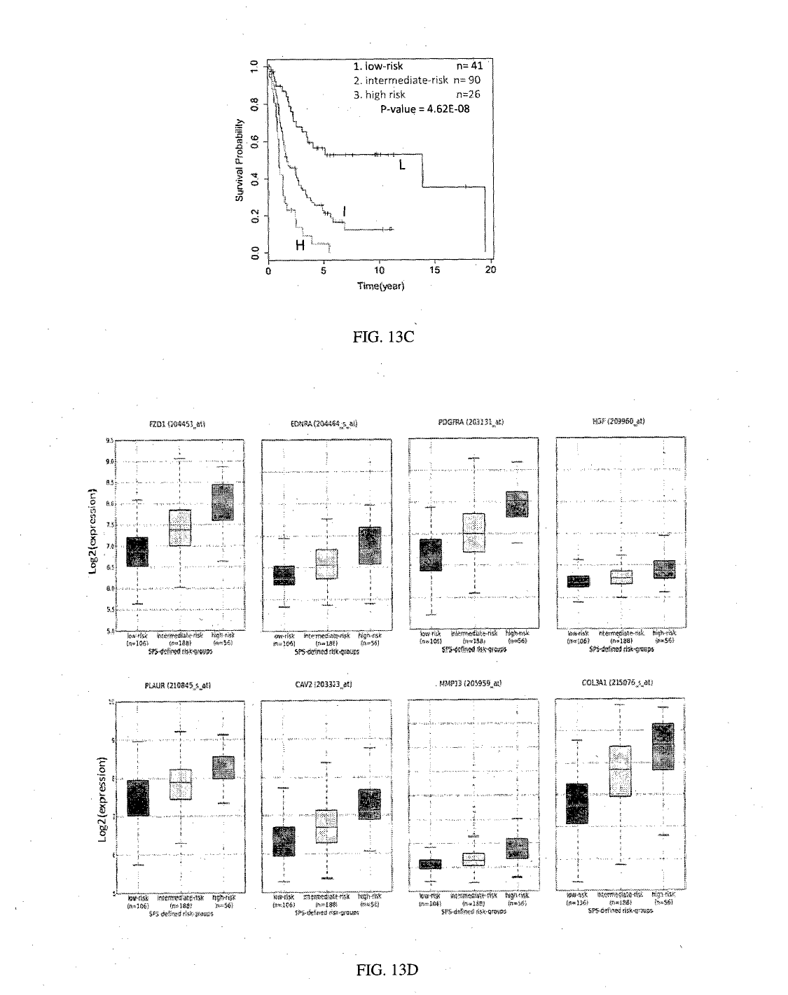

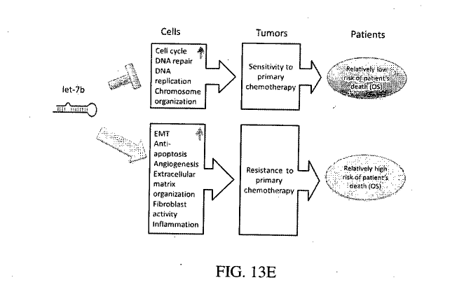

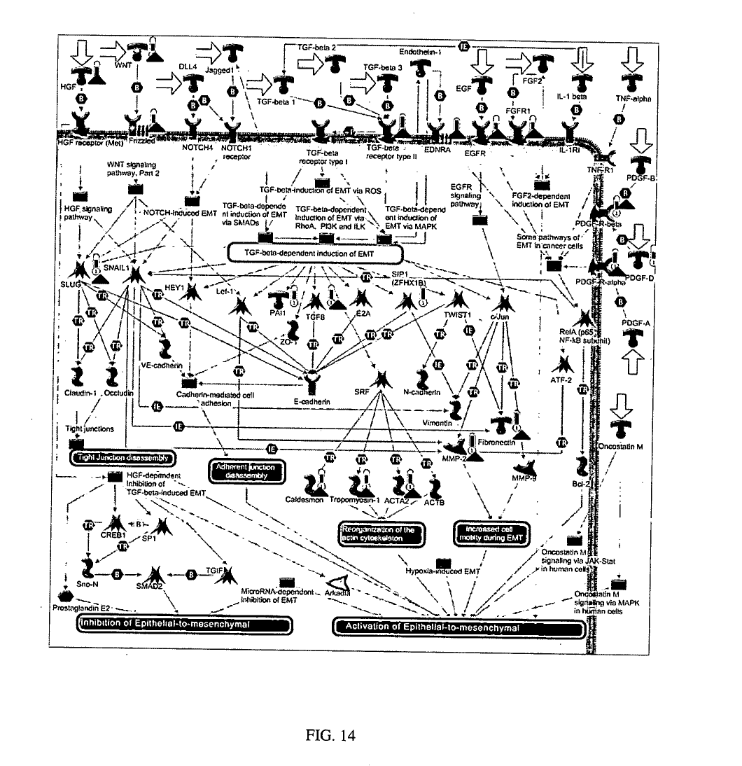

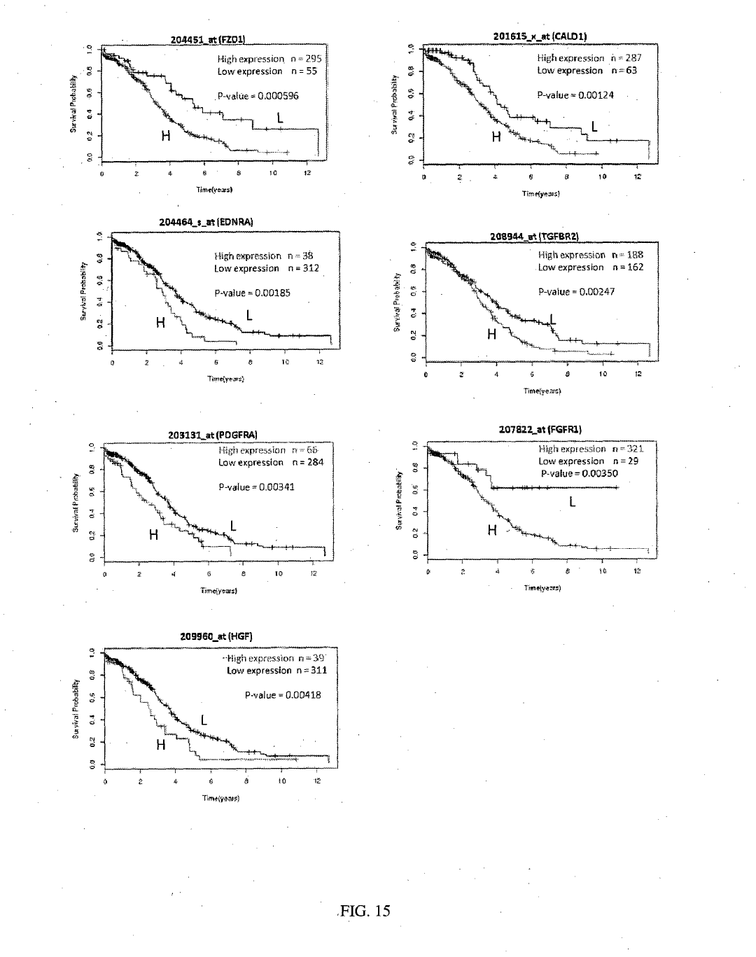

As will be described in more detail below, individual let-7 members exhibited diverse evolutionary, regulatory and functional characteristics (FIG. 1). Specifically, DDg analysis modified for the identification of three survival significant subgroups and k-means clustering of microarray miRNA expression signals revealed pro-oncogenic functions of let-7b and let-7c. Remarkably, the method we developed demonstrated that let-7b can display a dual synergistic master regulator activity which controls hundreds of genes involved in HG-EOC progression. The mRNA which significantly correlated with let-7b provided clear dichotomization of biological functions related to cancer progression. DDg-SWVg analysis revealed that a subset of 36 let-7b associated mRNAs could stratify HG-EOC patients into three distinct risk subgroups where the low-risk subgroup has a 5-year survival rate of 65-72%. In addition, a subset of 21 let-7b associated miRNAs could stratify HG-EOC patients into three distinct risk subgroups, where the low-risk subgroup has a 5-year survival rate of 53%. In a clinical setting, the 21-miRNA signature and/or 36-mRNA prognosis signature would be useful to clinicians during patient prognosis, prediction of primary therapy efficacy as well as the design of future personalized therapeutic intervention.

Thus, this methodological approach suggests the development of a novel class of combined biomarkers related to the regulatory pathways of pro-oncogenic agent let-7b. Let-7b associated 36-mRNA prognostic signature and 21-miRNA prognostic signature is clinically significant in HG-EOC, where the patients can be classified into one of low-, intermediate- or high-risk subgroups, with eventual implications on patient risk prognosis, assessment, management and patient therapy.

### Expression Datasets

TCGA datasets containing miRNA and mRNA expression profiles and clinical data of SOC samples were obtained through The Cancer Genome Atlas (TCGA) data portal (Cancer Genome Atlas Research Network, 2008). The TOGA miRNA dataset contains 13 batches of 520 samples in total, with 8-47 samples in each batch. Most of the patients (>90%) in this dataset were classified as stage III SOC. The miRNA expression data were generated using the Agilent Human miRNA Microarray Platform 8X15K, based on the Sanger miRBase (release 10.1). Agilent oligo 60-mer probes used in this platform were produced by SurePrint Technology. The microarray dataset was generated from the same patient reservoir as the miRNA dataset on an Affymetrix U133A platform, which contains 22,277 probe sets. This dataset contained 11 batches of 463 primary solid ovarian cancer tissue samples, with 21-47 samples in each batch.

A second miRNA dataset, generated in the Australian Ovarian Cancer Study (AOCS) by Shih et al. consisted of 62 microRNA samples generated from advanced SOC patients (stage III and IV) (Shih et al, 2011). This dataset was obtained from the Gene Expression Omnibus (GEO) website under accession number GSE27290 (http://www.ncbi.nlm.nih.gov/geo/). The Shih et al miRNA expression dataset was generated using the Agilent Human MicroRNA Microarray Platform 8X15K, V1.0 (beta version of G4470A) based on the Sanger Database, 9.1. The Agilent oligo 60-mer probes used in this platform were also produced by SurePrint Technology.

We evaluated the performance of our signature on three independent mRNA expression datasets obtained from GEO under accession numbers GSE9899 (Tothill et al, 2008), GSE26712 (Bonome et al, 2008), and GSE13876 (Crijns et al, 2009). In the GSE9899 dataset, 246 samples with Malignant Ser/PapSer were selected. Among them, 22 samples were in stage I/II, 222 were in stage III/IV, and 2 were of an unknown stage. Ninety-six samples were in grade 1/2, 148 samples were in grade 3, and 2 were of an unknown grade. GSE26712 and GSE13876 datasets contained 185 late-stage HG-OC samples and 157 advanced-stage SOC samples, respectively.

Currently, grading systems for OC are qualitative and rather subjective, with high intra- and inter-observer viability (Hernandez et al, 1984). As there are borderline differences between low grade (grade 1/2) and high grade (3/4) SOC in TCGA dataset, we included few samples (<10%) with grade 1 and grade 2 in TOGA and GSE9899 datasets.

### Pre-Processing and Quality Assessment

For each dataset, quality assessments were initially performed within each batch to identify poor quality chips. Background correction and normalization were then conducted within each batch. Finally, data from all batches were combined after batch effect adjustment.

For miRNA expression datasets, quality assessments were performed within each batch to identify poor quality chips, utilizing several visualization methods and statistical indicators on four typical signals from the Agilent platform (MeanSignal, ProcessedSignal, TotalProbeSignal, TotalGeneSignal). The statistical indicators were the median of log₂ intensity, log intensity ratio M (difference of log intensity), relative log expression (RLE), and correlation among samples, Box plot statistics were utilized to identify outliers for each of the above indicators in each signal. Density plots and MA plots were used to visualize the homogeneity of the data. Samples that failed in more than two indicators for more than two signals were identified as outliers and subsequently removed. The indicators were estimated again for the remaining samples. This procedure was performed iteratively, until no more outliers were present. Background correction and normalization were performed within each batch. We utilized invariant set normalization (ISN), in which a subset of probesets with small rank differences in their intensities in a series of arrays were selected to serve as references ad hoc as the basis for fitting a normalization curve. The fitted curve, the cubic smoothing spline to the probe intensities of these arrays, was used to calculate the correction to all probesets. The probe-level expression values were summarized by the median across arrays. Alternative normalization methods such as quantile normalization could also be used. Non-parametric ComBat software (http://jlab.byu.edu/ComBat/; Johnson et al., 2007) was utilized to correct for batch effects.

For the mRNA expression datasets, box plot statistics, MA plots and density plots were utilized to perform the outlier identification before pre-processing. In each batch, scale factor, average background, percentage of present call, GAPDH 3′:5′ ratio, GAPDH 3′:M ratio, Beta-actin 3′:5′ ratio, Beta-actin 3′:M ratio, slope of the RNA degradation plot, Normalized unscaled standard error (NUSE) median, NUSE IQR, Relative Log Expression (RLE) median, and RLE IQR were used as quality metrics. A sample was identified as an outlier if was an outlier with respect to more than two of these metrics. This procedure was performed iteratively, until no more samples could be identified as outliers. Following background correction and normalization, the Model-based expression index (MBEI) method was used to calculate probe set summaries. Other probe set summary methods such as RMA, or MAS5 or PLIER of Affymetrix are also possible. Analysis Of Variance (ANOVA)-based models (Kerr and Churchill, 2001) were adopted to correct possible batch effects in the microarray data.

Filtration of Unreliable miRNA and mRNA Microarray Probe-Sets

For the miRNA microarrays, the average expression of each of the 723 miRNA probesets was calculated across all arrays. Only 136 miRNA probesets were significantly expressed after setting a minimum untransformed (i.e., on the original scale) expression cut-off value of 25, based on the distribution of average miRNA probe expression.

For the mRNA microarray, the APMA database (Orlov et al, 2007) was used to remove unreliable probe-sets where discrepancies were found in annotation and target sequence mapping. Subsequently, using HGNC database (downloaded on 8 Dec. 2010), existing Affymetrix symbols were converted whenever possible to approved gene symbols, and Affymetrix probesets that did not map to an approved gene symbol were removed and unused in subsequent analysis. A total of 18,905 reliable Affymetrix probe-sets were retained.

### Data-Driven Grouping Survival Analysis

The Data-Driven grouping approach (DDg) for the two-group partitioning as described in Motakis et al. (2009) was applied to each dataset. In a generalization of DDg method, described in further detail below, a three-group partitioning of a patient cohort can be performed. DDg methods, whether they provide two-group or three-group partitioning, are based on fitting a semi-parametric Cox proportional-hazard regression model. The model was used to fit patients' overall survival (OS) times and events to gene expression data. The model estimates the optimal partition (cut-off) for the expression level of a gene by maximizing the separation of the survival curves related to the high- and low-risks of the disease behavior (for two subgroups partitioning), or low, intermediate and high-risks of the disease behavior (for three subgroups partitioning). The DDg method identifies single genes that exhibit a statistically significant influence on patients' survival or therapeutic outcome, and can divide patients into two or three distinct subgroups.

### A. Two Groups Partition Based on 1D DDg.

In this example, the 1D DDg method for feature selection procedure is used. Let the M×N matrix

$$X = {( {x}_{ij} )}_{{i = 1 , … , M}_{j = 1 , … , N}}$$

denote preprocessed expression data (as described above) for N genes in M patients. xᵢⱼ is the expression level of the j^{th} gene in the i^{th} patient. Let numeric array T=(tᵢ) denote the clinical outcome (survival time) of patients and nominal array E (eᵢ) denote the clinical event (1=deceased, 0=alive). For the j^{th} gene, let us rank-order the M patients according to the value of expression level of the gene. According to our model, in the case of unfavorable clinical outcome, a positive correlation between risk of death and gene expression level could be observed; alternatively, in the case of favorable clinical outcome, a negative correlation between risk of death and gene expression level could be observed. Assuming that the clinical outcomes are negatively (or positively) correlated with the expression of gene j, patient i can be separated into two subgroups (1=“high-risk”, 0=“low-risk”) at a pre-defined expression cutoff value cⱼ of the expression level of the j-th gene with the following formulae:

$${y}_{i}^{j} = \{ \begin{matrix}1 ( high - risk ) , & if {x}_{ij} > {c}_{j} \\ 0 ( low\_risk ) , & if {x}_{ij} \leq {c}_{j}\end{matrix} , \qquad ( 1 a )$$

in the case of unfavorable clinical outcome (positive correlation between risk of death and gene expression level), and

$${y}_{i}^{j} = \{ \begin{matrix}1 ( high - risk ) , & if {x}_{ij} \leq {c}_{j} \\ 0 ( low\_risk ) , & if {x}_{ij} > {c}_{j}\end{matrix} \qquad ( 1 b )$$

in the case of favorable clinical outcome (negative correlation between risk of death and gene expression level).

The survival curves corresponding to a favorable clinical outcome, given cutoff value cⱼ, can be described by K-M curves, characterizing a time-course of the probability of clinical outcome/events. The K-M curves could be fitted by a Cox proportional hazard regression model:

log hᵢ^{j}(tᵢ|yᵢ^{j},β^{j})=α^{j}+β^{j}·yᵢ^{j}, (2)

where hᵢ^{j} the hazard function, α^{j}=log hᵢ^{j}(t) represents the unspecified log-baseline hazard function when all of the y's are zero, and β^{j} is the regression parameter, and can be estimated by using the univariate Cox partial likelihood function:

$$L ( {β}^{j} ) = {\prod }_{i = 1}^{M} {\{ \frac{exp ( {β}^{i} {y}_{i}^{j} )}{{\sum }_{k ∈ R ( {t}_{i} )} exp ( {β}^{j} {y}_{k}^{j} )} \}}^{{e}_{i}} , \qquad ( 3 )$$

where R(tₖ)={k: tₖ≧tᵢ} is the risk set at time tᵢ.

For gene j at optimized cutoff value cⱼ, the Wald statistic (W) of the \hat{β}^{j} for each Cox proportional hazard regression model is estimated and serves as a measure of the subgroup discrimination. The genes with the largest βⁱ Wald Statistics (Wⱼ's) and having a p-value equal to or smaller than a predetermined threshold (typically, p-value ≦0.05) are considered. The method uses all potential predictors (e.g. all Affymetrix microarray probesets representing the expressed genes) as an input of the univariate or multivariate survival analysis. Our method processes these potential predictors/features and provides selection of the features as long as the p-value of the survival test statistic (e.g. the Wald statistic) for a given feature is equal to or less than the predetermined cut-off value (for instance, p≦0.05). The features providing p-values equal to or less than the cut-off value are picked up, rank-ordered by their p-value, and finally considered as the survival significant predictors.

Equations 1a and 1b suggest that the selection of prognostic-significant genes relies on the pre-defined expression cutoff value cⱼ of gene j based on which patients could be separated into two subgroups. A data-driven method (DDg) was developed to identify ‘the optimal’ cⱼ of gene j, which could ‘most successfully’ discriminate two subgroups corresponding to the minimum log-rank p-value with Wald estimation of β^{j}. The optimal value cⱼ of gene j provides a maximization of the difference between two K-M curves corresponding to the favorable and unfavorable clinical outcomes. The searching interval for optimal value cⱼ is defined between the 10^{th} quantile and 90^{th} quantile of the distribution of the signal intensity values for gene j. The detailed procedure can be found in the reference by Motakis et. al. (2009), the contents of which are incorporated by reference herein.

### B. Three Groups Partition Based on 1D DDg.

When 1D-DDg analysis is applied to separating three groups, two expression cutoffs of a mRNA or miRNA corresponding to local minimum p-values (e.g. corresponding to the Wald statistics) of a potential survival plot (left panel of FIG. 2) on the two deepest valleys of p-values of a survival curve plot could separate patients into three groups, as shown in FIG. 2. The cutoffs and p-values are obtained via fitting clinical outcomes/events to two patient groups by a Cox proportional hazard regression model. Assuming that the clinical outcomes are negatively correlated with the expression of mRNA or miRNA j, two cutoff values c₁ⱼ and c₂ⱼ (c₁ⱼ<c₂ⱼ) could be obtained which correspond to the local minima of two valleys in the curve of log(p-values) when comparing two groups separated by each cutoff value, and three groups could be found according to following equation, in which yᵢ^{j} is a group label for the i^{th} patient for mRNA or miRNA j:

$${y}_{i}^{j} = \{ \begin{matrix}1 ( high \text{-} risk ) & if {x}_{ij} > {c}_{2 j} \\ 0 ( intermediate \text{-} risk ) & if {c}_{1 j} < {x}_{ij} \leq {c}_{2 j} \\ - j ( low \text{-} risk ) & if {x}_{ij} \leq {c}_{1 j}\end{matrix} \qquad ( 4 )$$

Similar calculation procedures as in 1D-DDg could be applied. The data-driven “goodness-of-fit” method is utilized to identify the optimal cutoffs c₁ⱼ and c₂ⱼ of miRNA j, which could ‘most successfully’ discriminate three groups corresponding to two minimum values of the score estimated as a multiplication of three pairwise Wald p-values among three survival curves.

### Statistically-Weighted Voting Grouping (SWVg) Analysis

A Statistically weighted voting (SWVg) procedure based on DDg was utilized to obtain consensus grouping decisions from the grouping information generated by multiple covariates (e.g. microarray expressed genes).

A list of genes is ordered in ascending values according to their p-values generated from the DDg procedure above. The numeric grouping value for sample i could be calculated by the formula Gᵢ^{N}=Σⱼ₌₁^{N}wⱼGᵢⱼ, where N is the number of genes and Gᵢⱼ is the group allocation for sample i assigned by gene j in the DDg. The weight wⱼ is calculated by the formula

$${w}_{j} = \frac{- log ( {p}_{h} )}{{\sum }_{m = 1}^{N} ( - log ( {p}_{m} ) )} ,$$

where pⱼ is the p-value of gene j in the DDg procedure.

In a particular example where samples are divided into two groups, patient i could be separated into two subgroups (1=“high-risk”, 0=“low-risk”) at a pre-defined cutoff value (G_{C}) of Gᵢ^{N} with the following formula:

$${y}_{i}^{N} = \{ \begin{matrix}1 ( high \text{-} risk ) , & if {G}_{i}^{N} > {G}_{C} \\ 0 ( {low}_{risk} ) , & if {G}_{j}^{N} \leq {G}_{C}\end{matrix}$$

A Cox proportional hazard regression model is estimated by using a univariate Cox partial likelihood function with the method described in the DDg procedure.

Wald statistic of \hat{β}^{j} is estimated and serves as an indicator to evaluate the ability of group discrimination for gene j at cutoff G_{C}. The searching space of G_{C} is from 0.2 to 0.8, with an increment of 0.01 for each step. The G_{C} that provides the minimum log-rank p-values in the searching space is the optimized G_{C}. The above-described procedure is repeated for different N, which varies from 3 to the number of genes assigned. The number (Nₒₚₜ) and combination of genes are optimized for minimum log-rank p-values.

In a particular example where the samples are divided into three subgroups, two cutoff values (G_{C1}, G_{C2}, G_{C1}<G_{C2}) of yᵢ^{N} are calculated according to the following formula:

$${y}_{i}^{N} = \{ \begin{matrix}1 & ( high risk ) if {G}_{i}^{N} > {G}_{C 2} \\ 0 & ( intermediate risk ) if {G}_{C 1} < {G}_{1}^{N} \leq {G}_{C 2} \\ - 1 & ( low risk ) if {G}_{i}^{N} \leq {G}_{C 1}\end{matrix}$$

A Cox proportional hazard regression model and log-rank statistic estimates are computed. G_{C1} is searched in the range from 0.2 and 0.44, with an increment of 0.01 for each step; while G_{C2} is searched in the range from 0.56 to 0.8, with an increment of 0.01 for each step. G_{C1}, G_{C2} and Nₒₚₜ are optimized for the minimum value of multiplication of pair-wise log-rank p-values of 3 survival curves.

### Clustering Analysis of Let-7 Family Members' Expression

Open source clustering software Cluster 3.0 and visualization software Java Treeview (Eisen et al, 1998) were utilized to perform K-means clustering with k=3. Kendall tau correlation was used to measure the distance matrix. The Kaplan-Meier survival analysis was used to calculate the survival status of each cluster. The log-rank test was used to compare the survival distribution of the three samples.

### Gene Ontology Analysis

Gene ontology analyses were performed via DAVID Bioinformatics tools (Huang et al, 2009) and MetaCore™ (version 6.8 build 29806, from GeneGo Inc). In both analyses, the filtered list of 18,905 reliable Affymetrix probe-sets was uploaded as background to prevent any systematic bias during the statistical calculations. In DAVID Bioinformatics tools, categories of interest included OMIM, GO_BP_GAT, GO_CC_FAT, GO_MF_FAT, Panther_BP_AII, Panther_MF_AII, BBID, BIOCARTA, KEGG, Interpro, PIR_Superfamily, SMART and UP_TISSUE. In MetaCore, gene enrichment reports in curated pathways, processes, and diseases were generated.

### Differential Expression Analysis of the Patient Subgroups

From the let-7b-associated mRNA signatures comprising 36 genes, 350 patients from TCGA ovarian cancer database were able to be stratified into three distinct subgroups, where the low-, intermediate- and high-risk subgroups showed distinct 5-year survival rates of 64%, 12% and 10%, respectively. For each miRNA and mRNA probe, pair-wise differential expression was performed among the three subgroups, which contained 106, 188 and 56 patients in the low-, intermediate- and high-risk subgroups, respectively. The significances of the differential expression were calculated using non-parametric Mann-Whitney test and corrected for multiple probe testing (across all probsets in U133A platform) via the Benjamini-Hochberg Step-Up FDR method. Subsequently, for each pair of risk subgroup transition (i.e., low to intermediate-risk or high to low-risk), the differentially expressed probesets (FDR≦0.05) were extracted to perform gene ontology analysis.

### Cross Validation Analysis

To assess the stability of the groupings obtained via 1D DDg and SWVg, a ten-fold cross validation procedure can be performed as follows:
1) The patient cohort is first split into 10 distinct bins and 10 simulations are performed.
2) In each simulation, patients from one bin are used as the validation set, whereas the rest are used as the training set.
   a. For the training set, the patients are stratified into 2 or 3 risk subgroups based on optimized parameters of 1D DDg and SWVg.
   b. The optimized parameters derived from the training set of patients are then applied to the remaining bin of patients which has been designated as the validation set (10% of all patients). For each patient in the validation set, his/her gene expression profile is evaluated using the optimized 1D DDg parameters. Subsequently, the patient is assigned a predicted risk grouping (i.e. low, intermediate or high-risk) based on the optimized SWVg parameters.
   c. The analysis is repeated until all 10 patient bins have been used as the validation set.
3) After ten rounds of cross validation, the 10 validation grouping results are combined together to procedure a single grouping estimation of the whole samples.

Comparison of the patient grouping from ten-fold cross validation with the original DDg-SWVg provides strong indication that the parameters of 1D DDg and SWVg are stable, and can be applied reliably to independent patient or set of patients (Table 1, FIG. 3). SWVg provides strong indication that the parameters of 1D DDg and SWVg are stable. Results of cross-validation analysis presented in Table 1.

TABLE 1
Confusion matrix table (Overall accuracy: 73%)

| | | Grouping using all | | | Positive |
|---|---|---|---|---|---|
| | | samples by DDg-SWVg | | | predictive |
| | | 1 | 2 | 3 | value |
| Cross | 1 | 67 | 21 | 0 | 76% |
| validation | 2 | 40 | 163 | 32 | 69% |
| | 3 | 0 | 3 | 24 | 80% |

| | | | |
|---|---|---|---|
| sensitivity | 63% | 87% | 43% |

Comparison of the Let-7b-Associated 36-mRNA Prognosis Signature with Random Gene ID Lists

Prior to survival analyses, 162 Affymetrix U133A probesets correlated with let-7b and significantly associated with biological pathways were selected. For each of these 162 probesets, survival significance of the individual probeset was evaluated. Finally, via statistically-weighted voting, the let-7b-associated 36-mRNA prognosis signature comprising of the top 36 survival-significant genes were able to separate patients into three distinct risk subgroups of which the significance of separation is measured by a log-rank p-value.

To validate our biomarker selection methods, a set of negative control probes were defined as those that were not 1D DDg survival significant (p-value >0.1). From this set of negative control probesets, 999 probeset lists, each containing 162 probesets, were randomly generated without replacement within each list. Each list was generated independently from the list of negative control probesets. For each randomly generated list, similar 1D DDg and SWVg analyses were performed on the 162 probes to eventually generate the let-7b-associated 36-mRNA prognosis signature.

The log-rank p-value of our actual 36-mRNA prognosis signature was compared to the distribution of the random log-rank p-values.

### Correlation Analysis and Clustering Analysis

Tests on the associations of two miRNAs or miRNA-mRNA pairs were calculated using Kendall's tau correlation. To correct for multiple observations, we adjusted the P-value using Benjamini-Hochberg step-up FDR correction. Clustering analysis of the correlation coefficients of all of the combinations of let-7s and mRNA probes were performed. We extracted a subset of Affymetrix mRNA probe-sets that showed a strong correlation (FDR <0.01) for any of the let-7 members and performed hierarchical clustering analysis.

### Survival Significant Pathways Analysis

Pathway enrichment analyses were performed for positively and negatively correlated genes of let-7b independently. Pathways that were significantly associated with the positively and negatively correlated probes of let-7b (p-value <0.001) were generated by MetaCore. The expression values of specific genes were obtained from the probes with the most significant correlation with let-7b. The values were then used in an integrative analysis of the individual gene expression with the clinical data across all patients to examine the prognostic ability of each of these genes to predict HG-SOC patients' post-surgery survivability. Significant mRNAs were utilized in a SWVg procedure, where weights were assigned to the ranked list of DDg survival-significant genes to derive a representative gene signature to discriminate patients into low-, intermediate- and high-risk post-surgery treatment outcomes.

### Univariate, Multivariate Analyses and Kappa Correlation Test of Association

Univariate hazard ratios (HR) were calculated with 95-percent confidence intervals (95% CI) in Cox proportional-hazards model. Probabilities of overall survival (OS) were estimated by the Kaplan-Meier method, and the Wald test from the corresponding models was utilized to compare time-to-event distributions. Other co-variates included tumor stage, histologic grade, primary therapy outcome success, and tumor residual disease. The simultaneous prognostic effect of various factors was determined in a multivariate analysis in a Cox proportional-hazards model. The level of agreement between our predicted molecular subgroups and the clinical subgroups were evaluated by weighted Kappa correlation value (StatXact-9). The significance of the agreement was estimated by Mantel-Haenszel (MH) test (Agresti, 2007). All P-values are two-sided.

### Example 1

### Expression Patterns of Let-7 Family Members in HG-SOC can Classify Patients into Three Distinct Risk Subgroups

The reporting recommendations for tumor marker prognostic studies (REMARK; McShane et al, 2005) were adopted to identify potential biomarkers. We analyzed two independent miRNA expression datasets (TCGA and GSE27290, as discussed above) collected from HG-SOC patients (Tables 2 and 3).

TABLE 2
Clinical characteristics of The Cancer Genome Atlas
(TCGA) and GSE27290 datasets (OS: Overall survival)

| | | | |
|---|---|---|---|
| Survival | | average OS | average age |
| status | Recurrent status | (month) | (year) |

TCGA dataset

| | | |
|---|---|---|
| all 514 samples | 33.94 | 59.67 |

| | | | | |
|---|---|---|---|---|
| 223 | 81 | recurrent | 45.83 | 57.28 |
| alive | 139 | non-recurrent | 24.41 | 58.6 |
| | 3 | unknown | NA | 67.33 |
| 265 | 179 | recurrent | 41.66 | 59.93 |
| dead | 86 | non-recurrent | 22.45 | 63.42 |

GSE27290 dataset

| | | |
|---|---|---|
| all 49 samples | 50.25 | 63.01 |

| | | | | |
|---|---|---|---|---|
| 21 alive | 6 | recurrent | 80.98 | 59.79 |
| | 14 | no-recurrent | 73.58 | 64.42 |
| | 1 | unknown | 0.73 | 65.93 |
| 28 dead | 24 | recurrent | 35 | 61.14 |
| | 1 | non-recurrent | 87.03 | 75.33 |
| | 3 | unknown | 6.22 | 72.8 |

TABLE 3
Number and distribution of cases and relative survival rates of the TCGA dataset (486
primary solid tumor samples)

| | | median | | | | | | | |
|---|---|---|---|---|---|---|---|---|---|
| | | survival | Case (relative survival Rate (%) ) | | | | | | |
| | Cases | time ⁺ | <1 year | 1-year | 2-Year | 3-Year | 4-Year | >= 5-Year | Others ^{*} |
| Total | 486 | 2.43 | 48(9.9) | 48(9.9) | 54(11.1) | 48(9.9) | 29(6) | 70(14.4) | 189(38.9) |

Race

| | | | | | | | | | |
|---|---|---|---|---|---|---|---|---|---|
| white | 422 | 2.81 | 42(10) | 42(10) | 48(11) | 45(11) | 26(6) | 65(15) | 154(36) |
| others | 35 | 2.25 | 4(11) | 5(14) | 5(14) | 2(6) | 3(9) | 2(6) | 14(40) |
| unknown | 29 | 2.02 | 2(7) | 1(3) | 1(3) | 1(3) | 0(0) | 3(10) | 21(72) |

Age at initial pathologic diagnosis

| | | | | | | | | | | |
|---|---|---|---|---|---|---|---|---|---|---|
| <40 | Years | 16 | 3.95 | 0(0) | 1(6) | 2(13) | 1(6) | 0(0) | 3(19) | 9(56) |
| 40-60 | year | 248 | 3.08 | 14(6) | 23(9) | 22(9) | 26(10) | 22(9) | 34(14) | 107(43) |
| 60-80 | year | 200 | 2.30 | 33(17) | 22(11) | 29(15) | 19(10) | 7(4) | 31(16) | 59(30) |

| | | | | | | | | | |
|---|---|---|---|---|---|---|---|---|---|
| >80 | 15 | 2.02 | 1(7) | 1(7) | 1(7) | 2(13) | 0(0) | 2(13) | 8(53) |
| unknown | 7 | 1.54 | 0(0) | 1(14) | 0(0) | 0(0) | 0(0) | 0(0) | 6(86) |

Stages

| | | | | | | | | | |
|---|---|---|---|---|---|---|---|---|---|
| I | 14 | 0.36 | 2(14) | 0(0) | 0(0) | 0(0) | 0(0) | 2(14) | 10(71) |
| II | 21 | 3.69 | 0(0) | 1(5) | 1(5) | 3(14) | 1(5) | 7(33) | 8(38) |
| III | 366 | 2.9 | 32(9) | 40(11) | 44(12) | 41(11) | 23(6) | 49(13) | 137(37) |
| IV | 72 | 2.24 | 14(19) | 7(19) | 9(13) | 4(6) | 4(6) | 12(17) | 22(31) |
| unknown | 13 | 2.69 | 0(0) | 0(0) | 0(0) | 0(0) | 1(8) | 0(0) | 12(92) |

Grade

| | | | | | | | | | |
|---|---|---|---|---|---|---|---|---|---|
| 1 | 4 | 5.38 | 0(0) | 0(0) | 0(0) | 0(0) | 1(25) | 1(25) | 2(50) |
| 2 | 57 | 3.47 | 3(5) | 2(4) | 8(14) | 9(16) | 3(5) | 19(33) | 13(23) |
| 3 | 410 | 2.71 | 44(11) | 45(11) | 45(11) | 35(9) | 24(6) | 49(12) | 168(41) |
| 4 | 1 | 3.67 | 0(0) | 0(0) | 0(0) | 1(100) | 0(0) | 0(0) | 0(0) |
| unknown | 14 | 3.25 | 1(7) | 1(7) | 1(7) | 3(21) | 1(7) | 1(7) | 8(57) |

Chemotherapy

| | | | | | | | | | |
|---|---|---|---|---|---|---|---|---|---|
| Yes | 439 | 2.92 | 28(6) | 44(10) | 50(11) | 47(11) | 28(6) | 65(15) | 177(40) |
| no | 23 | 0.18 | 13(57) | 1(4) | 1(4) | 1(4) | 1(4) | 2(9) | 4(17) |
| unknown | 24 | 0.89 | 7(29) | 3(13) | 3(13) | 0(0) | 0(0) | 3(13) | 8(33) |

Primary therapy outcome success

| | | | | | | | | | |
|---|---|---|---|---|---|---|---|---|---|
| complete_response | 270 | 3.63 | 4(1) | 15(6) | 24(9) | 31(11) | 19(7) | 61(23) | 116(43) |
| partial_response | 56 | 2.39 | 4(7) | 14(25) | 13(23) | 4(7) | 5(9) | 3(5) | 13(23) |
| progressive_disease | 36 | 1.75 | 9(25) | 6(17) | 6(17) | 5(14) | 2(6) | 1(3) | 7(19) |
| stable_disease | 23 | 2.60 | 3(13) | 3(13) | 3(13) | 1(4) | 1(4) | 2(9) | 10(43) |
| unknown | 101 | 1.25 | 28(28) | 10(10) | 8(8) | 7(7) | 2(2) | 3(3) | 43(43) |

Site of tumor first recurrence

| | | | | | | | | | |
|---|---|---|---|---|---|---|---|---|---|
| loco-regional | 124 | 3.02 | 6(5) | 18(15) | 18(15) | 21(17) | 13(10) | 20(16) | 28(23) |
| metastasis | 118 | 3.17 | 5(4) | 15(13) | 18(15) | 17(14) | 12(10) | 21(18) | 30(25) |
| unknown | 244 | 1.49 | 37(15) | 15(6) | 18(7) | 10(4) | 4(2) | 29(12) | 131(54) |

Tumor residual disease

| | | | | | | | | | | |
|---|---|---|---|---|---|---|---|---|---|---|
| >20 | mm | 79 | 2.08 | 8(10) | 13(16) | 11(14) | 5(6) | 3(4) | 12(15) | 27(34) |
| 11-20 | mm | 26 | 2.79 | 4(15) | 2(8) | 4(15) | 3(12) | 1(4) | 5(19) | 7(27) |
| 1-10 | mm | 212 | 2.87 | 20(9) | 24(11) | 32(15) | 29(14) | 16(8) | 22(10) | 69(33) |

| | | | | | | | | | |
|---|---|---|---|---|---|---|---|---|---|
| no_macroscopic_disease | 95 | 3.21 | 7(7) | 4(4) | 4(4) | 7(7) | 5(5) | 15(16) | 53(56) |
| unknown | 74 | 2.60 | 9(12) | 5(7) | 3(4) | 4(5) | 4(5) | 16(22) | 33(45) |

Anatomic organ: subdivision

| | | | | | | | | | |
|---|---|---|---|---|---|---|---|---|---|
| bilateral | 323 | 2.84 | 31(10) | 37(11) | 34(11) | 34(11) | 24(7) | 43(13) | 120(37) |
| left | 67 | 2.87 | 5(7) | 4(6) | 9(13) | 7(10) | 2(3) | 15(22) | 25(37) |
| right | 46 | 2.43 | 9(20) | 2(4) | 6(13) | 4(9) | 2(4) | 4(9) | 19(41) |
| unknown | 50 | 2.20 | 3(6) | 5(10) | 5(10) | 3(6) | 1(2) | 8(16) | 25(50) |

Person neoplasm cancer status

| | | | | | | | | | |
|---|---|---|---|---|---|---|---|---|---|
| tumor_free | 112 | 4.62 | 1(1) | 0(0) | 0(0) | 0(0) | 3(3) | 24(21) | 84(75) |
| with_tumor | 308 | 2.81 | 34(11) | 45(15) | 48(16) | 41(13) | 25(8) | 40(13) | 75(24) |
| unknown | 66 | 2.52 | 13(20) | 3(5) | 6(9) | 7(11) | 1(2) | 6(9) | 30(45) |

Venous invasion

| | | | | | | | | | |
|---|---|---|---|---|---|---|---|---|---|
| yes | 72 | 2.89 | 5(7) | 4(6) | 9(13) | 3(4) | 4(6) | 12(17) | 35(49) |
| no | 68 | 2.58 | 6(9) | 5(7) | 6(9) | 6(9) | 2(3) | 11(16) | 32(47) |
| unknown | 346 | 2.81 | 37(11) | 39(11) | 39(11) | 39(11) | 23(7) | 47(14) | 122(35) |

Lymphatic invasion

| | | | | | | | | | |
|---|---|---|---|---|---|---|---|---|---|
| yes | 109 | 2.62 | 13(12) | 10(9) | 14(13) | 6(6) | 6(6) | 11(10) | 49(45) |
| no | 74 | 2.65 | 7(9) | 5(7) | 5(7) | 7(9) | 4(5) | 12(16) | 34(46) |
| unknown | 303 | 2.83 | 28(9) | 33(11) | 35(12) | 35(12) | 19(6) | 47(16) | 106(35) |

⁺ median survival time is calculated from the information of the deceased patients only
^{*} Alive patients with follow-up <5 years or patient with no follow-up information

After removing outlier samples, 514 profiles in TCGA dataset, and 49 profiles in GSE27290 qualified for the analysis (FIG. 4). We found that the relative expression level of let-7 family members were higher than many other miRNAs in the studied cancer samples. DDg coupled with SWVg and k-means cluster analyses were performed on the expression profiles of both datasets (Tables 4 and 5). Table 4 contains information about p-values and cutoff values for individual miRNAs of let-7 miRNA family and p-value score of SWVg. The same list of let-7 miRNA family members could provide significant partition of the patients taken from GSE27290 dataset (p-value=0.00000385).

TABLE 4
The parameters and P-values generated from DDg and p-
value from SWVg analysis in TCGA dataset

| | | | p-value; | Statistical- |
|---|---|---|---|---|
| | | | Data-driven | weighted |
| | | | grouping | voting |
| miRNAs | Cutoff | Design* | procedure | prognosis |
| hsa-miR-98 | 4.70 | 1 | 1.49E−04 | 9.48E−07 |
| hsa-let-7f | 7.83 | 1 | 1.44E−03 | |
| hsa-let-7g | 6.91 | 1 | 1.94E−03 | |
| hsa-let-7a | 7.60 | 1 | 2.35E−03 | |
| hsa-let-7b | 8.50 | 2 | 5.30E−03 | |
| hsa-let-7e | 6.77 | 1 | 5.39E−03 | |
| hsa-let-7c | 7.18 | 2 | 1.03E−02 | |
| hsa-let-7d | 6.35 | 1 | 1.31E−02 | |
| hsa-let-7i | 6.60 | 1 | 9.98E−02 | |

*1: pro-tumor suppressor; 2: pro-oncogene

TABLE 5
Confusion matrix of the group information acquired
from SWV and k-means clustering analysis. The
number of samples that were consistently grouped into
same groups by both methods is highlighted in bold font.

| Kmeans clustering | | | |
|---|---|---|---|
| Low risk | intermediate risk | high risk | total |

TCGA dataset

| | | | | | |
|---|---|---|---|---|---|
| SWV | Low risk | 238 | 0 | 0 | 238 |
| | intermediate | 0 | 191 | 0 | 191 |
| | risk | | | | |
| | high risk | 2 | 2 | 32 | 36 |
| | total | 240 | 193 | 32 | 465 |

TCGA27290 dataset

| | | | | | |
|---|---|---|---|---|---|
| SWV | Low risk | 12 | 6 | 0 | 18 |
| | intermediate | 7 | 12 | 6 | 25 |
| | risk | | | | |
| | high risk | 2 | 1 | 3 | 6 |
| | Total | 21 | 19 | 9 | 49 |

For the GSE27290 dataset, 49 samples were separated into three risk subgroups (low-, intermediate- and high-risk), and 27 of these samples (55%) were clustered consistently by the two methods (Table 5). The log-rank test showed significant differences in the OS among the three subgroups. Specifically, the expressions of let-7b and let-7c were higher in the high-risk subgroup as compared with that in the low-risk subgroup. In contrast, the expression levels of let-7a, let-7f and let-7g were lower in both high- and intermediate-risk subgroups as compared with those in the low-risk subgroup. Similar sub-groupings and results were obtained by analyzing the samples in TCGA dataset. The expression of let-7b and let-7c were higher in the high-risk subgroup than that in the low-risk subgroup, suggesting unfavorable influences of both miRNAs on post-surgery treatment responses of HG-SOC patients (FIG. 5). In contrast, the expressions of let-7a and let-7f in the low-risk subgroup were significantly higher than those in the high-risk subgroup. The consistent results obtained from two independent datasets using two distinct unsupervised approaches suggest that HG-SOC may contain three distinct molecular and clinical tumor subtypes, and that an elevation of let-7b and let-7c expression in HG-SOC may lead to disease progression and poor post-surgery treatment outcome.

Furthermore, we utilized an online tool MIRUMIR (Antonov et al., 2012; www.bioprofiling.de/GEO/MIRUMIR/mirumir.html) to assess the relationship between expression levels of let-7 members with clinical outcomes (particularly, OS) and found that let-7b and let-7c have different functions in different cancer types. The higher expression levels were associated with relatively poor prognosis for HG-SOC patients, relatively good prognosis for breast cancer patients and no survival significance among prostate cancer patients (FIG. 6). While previous publications have reported that let-7 family members in OC are expressed at lower levels than in normal ovarian epithelial tissue (Nam et al, 2008; Yang et al, 2008), there are seldom reports comparing their functions in different risk subtypes of HG-SOC, which is the objective of our study.

### Example 2

### Let-7b as a Master Regulator in HG-SOC with Dichotomization of Patho-Biological Functions

A correlation analysis of miRNA expression between let-7 members for both datasets (FIG. 7) indicated that the expression of miR-202 was negatively correlated with the other members; this suggested that it is an outlier within this family. The expression levels of let-7b and let-7c, while significantly and positively correlated with each other, were less correlated with other let-7 members, which were significantly and positively correlated. An analysis of the sequence and co-expression patterns of let-7b and let-7c indicated their grouping in one distinct cluster and hinted toward their similar functions in HG-SOC.

Hierarchical clustering analysis was performed on the correlation coefficients of let-7 with 141 miRNAs present in both TCGA and GSE27290 datasets (FIG. 8). Let-7b and let-7c shows different pattern with other members. Of the 141 miRNA, 103 miRNA (73%) were in the same clusters in both datasets. In particular, we found 21 miRNAs, whose expression levels showed correlations with all of the let-7 family members in both datasets. SWVg analysis revealed that the 21 miRNAs consists of a high-confidence prognostic signature stratifying patients into three distinct survival subclasses. Besides, in both datasets the 21 miRNAs form two groups, reflecting a cluster structure of the let-7 family (FIGS. 8C and 8D). Among them, four miRNAs (hsa-miR-22, hsa-miR-214, hsa-miR-127, hsa-miR-136) were significantly positive-correlated, while three (hsa-miR-103, hsa-miR-106b, hsa-miR-96) were significantly negative-correlated with let-7b in both TCGA and GSE27290 datasets.

To achieve an understanding of the correlation patterns of the miRNAs across the genome, we performed correlation analysis between miRNA and mRNA probesets represented in the TCGA microarray datasets, and identified classes of protein-coding genes potentially controlled by the let-7 family. For each member, the distribution curves of correlation coefficients with all mRNA probes were compared with the background distribution. The correlation pattern associated with let-7b was distinct from the background distribution for all miRNA-mRNA pairs. Specifically, the frequency distribution of the correlation coefficients for let-7b had a wider profile, suggesting that let-7b was strongly correlated with a large number of mRNAs in the HG-SOC genome (FIG. 9A).

In total, the expression levels of 4,126 Affymetrix U133A probesets were significantly correlated with the expression levels of any of the let-7 family members (FDR<0.01, FIG. 10). Among them, 2,971 (72%) probesets were due to let-7b. Hierarchical clustering analysis of the correlation coefficients of the 4,126 probesets and let-7 signals revealed two distinct clusters for the mRNA probesets that were significantly correlated with let-7b expression signal. Let-7b, let-7c and let-7d exhibited similar correlation patterns with the mRNAs, but the correlations of let-7b were significantly stronger. Analysis of the mRNAs in the two clusters via gene ontology (GO) analysis revealed that the two sets of genes were remarkably enriched with entirely distinct gene functions (FIG. 9B). Positively correlated mRNA-miRNA pairs were significantly associated with EMT and ECM-receptor interactions, while negatively correlated mRNA-miRNA pairs were associated with cell cycle-related functions.

To investigate whether mRNAs correlated with let-7b could be significantly enriched in any biological pathways, we performed enrichment analysis using MetaCore (FIG. 9). From 1514 probesets that were positively correlated with let-7b (FDR <0.01), 116 unique probesets were significantly enriched in six pathways including immune response, ECM remodeling, chemokines, adhesion and the regulation of EMT pathway (P-value <0.001, FIG. 9C, Table 6).

TABLE 6
Significant pathway maps of mRNA probes positively correlated with let-7b (FDR < 0.01). 116 unique probesets correlated with expression let-7b are significantly enriched in six pathways including immune
response/classical complement and alternative complement pathways, ECM remodeling, chemokines, adhesion and the regulation of EMT pathway.

| | | In List | | | | | In Background | | |
|---|---|---|---|---|---|---|---|---|---|
| | | # | | | | | # | | |
| | | metacore | # gene | gene | # | | metacore | # gene | gene |
| Maps | pValue | objects | symbols | symbols | probes | probes | objects | symbols | symbols |
| Immune response | 7.31E−07 | 15 | 13 | C1R, C1S, C2, C3, C4A, C4B, | 14 | 200985_s_at, 201925_s_at, | 30 | 48 | C1QA, C1QB, C1QC, C1R, C1S, C2, C3, |
| Classical complement | | | | CD55, CD59, CD93, CLU, ITGAM, | | 201926_s_at, 202803_s_at, | | | C3AR1,C4A, C4B, C4BPA, C4BPB, C5, |
| pathway | | | | ITGAX, ITGB2 | | 202877_s_at, 202878_s_at, | | | C5AR1, C6, C7, C8A, C8B, C8G, C9, |
| | | | | | | 203052_at, 205786_s_at, | | | CD46, CD55, CD59, CD93, CFI, CLU, |
| | | | | | | 208747_s_at, 208791_at, | | | CR1, CR2, CRP, IGH@, IGHD@, IGHG1, |
| | | | | | | 210184_at, 212067_s_at, | | | IGHJ@, IGHM, IGHV3-23, IGHV@, IGK@, |
| | | | | | | 214428_x_at, 217767_at | | | IGKC, IGKJ@, IGKV@, IGL@, IGLC@, |
| | | | | | | | | | IGLJ@, IGLV@, ITGAM, ITGAX, ITGB2, |
| | | | | | | | | | SERPING1 |
| Immune response | 1.74E−06 | 14 | 10 | C3, CD55, CD59, CFB, CFD, CFH, | 11 | 200985_s_at, 201925_s_at, | 28 | 24 | C3, C3AR1, C5, C5AR1, C6, C7, C8A, |
| Alternative complement | | | | CLU, ITGAM, ITGAX, ITGB2 | | 201926_s_at, 202357_s_at, | | | C8B, C8G, C9, CD46, CD55, CD59, CFB, |
| pathway | | | | | | 202803_s_at, 205382_s_at, | | | CFD, CFH, CFI, CFP, CLU, CR1, CR2, |
| | | | | | | 205786_s_at, 208791_at, | | | ITGAM, ITGAX, ITGB2 |
| | | | | | | 210184_at, 215388_s_at, | | | |
| | | | | | | 217767_at | | | |
| Cell adhesion_ECM | 1.59E−05 | 17 | 22 | CD44, COL1A1, COL1A2, COL3A1, | 45 | 200600_at, 200665_s_at, | 45 | 61 | CD44, COL1A1, COL1A2, COL2A1, |
| remodeling | | | | EGFR, FN1, HBEGF, IGFBP4, | | 201069_at, 201148_s_at, | | | COL3A1, COL4A1, COL4A2, COL4A3, |
| | | | | ITGA5, LAMA3, LAMA4, LAMC2 | | 201149_s_at, 201150_s_at, | | | COL4A4, COL4A5, COL4A6, CXCR1, |
| | | | | MMP13, MMP2, MSN, NID1, PLAU, | | 201389_at, 201508_at, | | | EGFR, ERBB4, EZR, FN1, HBEGF, IGF1, |
| | | | | PLAUR, SERPINE1, SPARC, | | 201852_x_at, 201983_s_at, | | | IGF1R, IGF2, IGFBP4, IL8, ITGA1, ITGA5, |
| | | | | TIMP3, VCAN | | 202007_at, 202202_s_at, | | | ITGB1, KLK1, KLK2, KLK3, LAMA1, |
| | | | | | | 202267_at, 202310_s_at, | | | LAMA3, LAMA4, LAMB1, LAMB3, LAMC1, |
| | | | | | | 202311_s_at, 202403_s_at, | | | LAMC2, MMP1, MMP10, MMP12, MMP13, |
| | | | | | | 202404_s_at, 202627_s_at, | | | MMP14, MMP15, MMP16, MMP2, MMP3, |
| | | | | | | 202628_s_at, 203726_s_at, | | | MMP7, MMP9, MSN, NID1, PLAT, PLAU, |
| | | | | | | 204489_s_at, 204490_s_at, | | | PLAUR, PLG, SDC2, SERPINE1, |
| | | | | | | 204619_s_at, 204620_s_at, | | | SERPINE2, SPARC, TIMP1, TIMP2, |
| | | | | | | 205479_s_at, 205959_at, | | | TIMP3, VCAN, VTN |
| | | | | | | 209835_x_at, 210495_x_at, | | | |
| | | | | | | 210845_s_at, 211571_s_at, | | | |
| | | | | | | 211668_s_at, 211719_x_at, | | | |
| | | | | | | 211924_s_at, 212014_x_at, | | | |
| | | | | | | 212063_at, 212464_s_at, | | | |
| | | | | | | 214701_s_at, 214702_at, | | | |
| | | | | | | 215076_s_at, 215646_s_at, | | | |
| | | | | | | 216442_x_at, 217430_x_at, | | | |
| | | | | | | 217523_at, 221731_x_at, | | | |
| | | | | | | 38037_at | | | |
| Immune response_Lectin | 4.48E−05 | 13 | 11 | C2, C3, C4A, C4B, CD55, CD59, | 12 | 200985_s_at, 201925_s_at, | 31 | 32 | C2, C3, C3AR1, C4A, C4B, C4BPA, |
| induced complement | | | | CD93, CLU, ITGAM, ITGAX, ITGB2 | | 201926_s_at, 202803_s_at, | | | C4BPB, C5, C5AR1, C6, C7, C8A, C8B, |
| pathway | | | | | | 202877_s_at, 202878_s_at, | | | C8G, C9, CD46, CD55, CD59, CD93, CFI, |
| | | | | | | 203052_at, 205786_s_at, | | | CLU, CR1, CR2, FCN2, FCN3, ITGAM, |
| | | | | | | 208791_at, 210184_at, | | | ITGAX, ITGB2, MASP1, MASP2, MBL2, |
| | | | | | | 214428_x_at, 217767_at | | | SERPING1 |
| Cell adhesion_Chemokines | 1.83E−04 | 20 | 32 | ACTA2, ACTN1, AKT3, ARPC1B, | 58 | 200600_at, 200859_x_at, | 68 | 154 | ACTA1, ACTA2, ACTB, ACTC1, ACTG1, |
| and adhesion | | | | CAV2, CCL2, CCR1, CD44, | | 200931_s_at, 200974_at, | | | ACTG2, ACTN1, ACTN2, ACTN3, ACTN4, |
| | | | | COL1A1, COL1A2, CXCL1, FLNA, | | 201040_at, 201069_at, | | | ACTR2, ACTR3, ACTR3B, AKT1, AKT2, |
| | | | | FN1, GNAI2, GNG12, GNG7, ILK, | | 201108_s_at, 201109_s_at, | | | AKT3, ARPC1A, ARPC1B, ARPC2, |
| | | | | ITGA3, ITGB4, LAMA4, LIMK2, | | 201110_s_at, 201234_at, | | | ARPC3, ARPC4, ARPC5, BCAR1, BRAF, |
| | | | | MAPK3, MMP13, MMP2, MSN, | | 201474_s_at, 201954_at, | | | CAV1, CAV2, CCL2, CCR1, CD44, CD47, |
| | | | | PIK3CG, PIK3R1, PLAU, PLAUR, | | 202193_at, 202202_s_at, | | | CDC42, CFL1, CFL2, COL1A1, COL1A2, |
| | | | | SERPINE1, THBS1, VCL | | 202310_s_at, 202311_s_at, | | | COL4A1, COL4A2, COL4A3, COL4A4, |
| | | | | | | 202403_s_at, 202404_s_at, | | | COL4A5, COL4A6, CRK, CTNNB1, |
| | | | | | | 202627_s_at, 202628_s_at, | | | CXCL1, CXCL5, CXCL6, CXCR1, CXCR2, |
| | | | | | | 203323_at, 203324_s_at, | | | DBN1, DOCK1, FLNA, FLOT2, FN1, |
| | | | | | | 204470_at, 204489_s_at, | | | GNAI1, GNAI2, GNAI3, GNAO1, GNAZ, |
| | | | | | | 204490_s_at, 204989_s_at, | | | GNB1, GNB2, GNB3, GNB4, GNB5, |
| | | | | | | 204990_s_at, 205098_at, | | | GNG10, GNG11, GNG12, GNG13, GNG2, |
| | | | | | | 205479_s_at, 205959_at, | | | GNG3, GNG4, GNG5, GNG7, GNG8, |
| | | | | | | 206370_at, 206896_s_at | | | GNGT1, GNGT2, GRB2, GSK3B, HRAS, |
| | | | | | | 208636_at, 208637_x_at | | | IL8, ILK, ITGA11, ITGA3, ITGA6, ITGA8, |
| | | | | | | 209835_x_at, 210495_x_at, | | | ITGAV, ITGB1, ITGB4, JUN, KDR, LAMA1, |
| | | | | | | 210582_s_at, 210845_s_at, | | | LAMA4, LAMB1, LAMC1, LEF1, LIMK1, |
| | | | | | | 211160_x_at, 211668_s_at, | | | LIMK2, MAP2K1, MAP2K2, MAPK1, |
| | | | | | | 211719_x_at, 211905_s_at, | | | MAPK3, MMP1, MMP13, MMP2, MSN, |
| | | | | | | 211924_s_at, 212014_x_at, | | | MYC, NFKB1, NFKB2, PAK1, PIK3CA, |
| | | | | | | 212046_x_at, 212063_at, | | | PIK3CB, PIK3CD, PIK3CG, PIK3R1, |
| | | | | | | 212239_at, 212294_at, | | | PIK3R2, PIK3R3, PIK3R5, PIP5K1C, |
| | | | | | | 212464_s_at, 212607_at, | | | PLAT, PLAU, PLAUR, PLG, PTEN, PTK2, |
| | | | | | | 213746_s_at, 214701_s_at, | | | PXN, RAC1, RAF1, RAP1A, RAP1GAP, |
| | | | | | | 214702_at, 214752_x_at, | | | REL, RELA, RELB, RHOA, ROCK1, |
| | | | | | | 216442_x_at, 216598_s_at, | | | ROCK2, SDC2, SERPINE1, SERPINE2, |
| | | | | | | 217430_x_at, 217523_at | | | SHC1, SOS1, SOS2, SRC, TCF7, |
| | | | | | | | | | TCF7L1, TCF7L2, THBS1, TLN1, TLN2, |
| | | | | | | | | | TRIO, VAV1, VCL, VEGFA, VTN, WASL, |
| | | | | | | | | | ZYX |
| Development_Regulation | 7.22E−04 | 17 | 19 | ACTA2, CALD1, EDNRA, EGFR, | 34 | 200974_at, 201069_at, | 59 | 90 | ACTA2, ACTB, ATF2, BCL2, CALD1, |
| of epithelial-to- | | | | FGFR1, FN1, FZD1, HGF, MMP2, | | 201615_x_at, 201616_s_at, | | | CDH1, CDH2, CDH5, CLDN1, CREB1, |
| mesenchymal transition | | | | PDGFD, PDGFRA, PDGFRB; | | 201617_x_at, 201983_s_at, | | | DLL4, EDN1, EDNRA, EGF, EGFR, FGF2, |
| (EMT) | | | | SERPINE1, SNAI2, TGFBR2, TPM1, | | 202273_at, 202627_s_at, | | | FGFR1, FN1, FZD1, FZD10, FZD2, FZD3, |
| | | | | WNT7A, ZEB1, ZEB2 | | 202628_s_at, 203131_at, | | | FZD4, FZD5, FZD6, FZD7, FZD8, FZD9, |
| | | | | | | 203603_s_at, 204451_at, | | | HEY1, HGF, IL1B, IL1R1, JAG1, JUN, |
| | | | | | | 204463_s_at, 204464_s_at, | | | LEF1, MET, MMP2, MMP9, NOTCH1, |
| | | | | | | 207822_at, 208944_at, | | | NOTCH4, OCLN, OSM, PDGFA, PDGFB, |
| | | | | | | 209960_at, 210248_at, | | | PDGFD, PDGFRA, PDGFRB, RELA, |
| | | | | | | 210495_x_at, 210986_s_at, | | | RNF111, SERPINE1, SKIL, SMAD2, |
| | | | | | | 210987_x_at, 211719_x_at, | | | SNAI1, SNAI2, SP1, SRF, TCF3, TGFB1, |
| | | | | | | 212077_at, 212464_s_at, | | | TGFB2, TGFB3, TGFBR1, TGFBR2, |
| | | | | | | 212758_s_at, 212764_at, | | | TGIF1, TJP1, TNF, TNFRSF1A, TPM1 , |
| | | | | | | 213139_at, 214701_s_at, | | | TWIST1, VIM, WNT1, WNT10A, WNT10B, |
| | | | | | | 214702_at, 214880_x_at, | | | WNT11, WNT16, WNT2, WNT2B, WNT3, |
| | | | | | | 215305_at, 216235_s_at, | | | WNT3A, WNT4, WNT5A, WNT5B, WNT6, |
| | | | | | | 216442_x_at, 219304_s_at | | | WNT7A, WNT7B, WNT8A, WNT8B, |
| | | | | | | | | | WNT9A, WNT9B, ZEB1, ZEB2 |

In contrast, from 1457 probesets that were negatively correlated with let-7b (FDR <0.01), 122 unique probesets were significantly enriched in eleven pathways associated with processes such as cell cycle regulation, metaphase checkpoints, DNA replication start, damage and DNA repair, role of BRCA1 and BRCA2 in DNA repair, spindle assembly, role of APC in cell cycle regulation, chromosome separation and condensation, apoptosis and survival (P-value<0.001, FIG. 9B, Table 7).

TABLE 7
Significant pathway maps of mRNA probes negatively correlated with let-7b (FDR < 0.01). 122 unique probesets are significantly enriched in eleven pathways associated with processes such as cell cycle
regulation, metaphase checkpoints, DNA replication start, damage and DNA repair, role of BRCA1 and BRCA2 in DNA repair, spindle assembly, role of APC in cell cycle regulation, chromosome
separation and condensation, apoptosis and survival

| | | In List | | | | | In Background | | |
|---|---|---|---|---|---|---|---|---|---|
| | | # | | | | | # | # | |
| | | metacore | # gene | gene | # | | metacore | gene | gene |
| Maps | pValue | objects | symbols | symbols | probesets | probesets | objects | symbols | symbols |
| Cell cycle_Role of | 8.36E−11 | 14 | 19 | ANAPC5, AURKA, AURKB, | 30 | 200098_s_at, 201327_s_at, 201897_s_at, | 22 | 54 | ANAPC1, ANAPC10, ANAPC11, |
| APC in cell cycle | | | | BUB1, BUB1B, CCNA2, CCT2, | | 201946_s_at, 201947_s_at, 203362_s_at, | | | ANAPC13, ANAPC2, ANAPC4, |
| regulation | | | | CCT6A, CDC25A, CDC6, | | 203418_at, 203625_x_at, 203755_at, | | | ANAPC5, ANAPC7, AURKA, |
| | | | | CDCA3, CDK2, CKS1B, | | 203968_s_at, 204092_s_at, 204252_at, | | | AURKB, BUB1, BUB1B, BUB3, |
| | | | | FBXO5, GMNN, MAD2L1, | | 204641_at, 204695_at, 208079_s_at, | | | CCNA1, CCNA2, CCNB1, |
| | | | | NEK2, SKP2, TCP1 | | 208080_at, 208721_s_at, 208722_s_at, | | | CCNB2, CCNB3, CCT2, CCT3, |
| | | | | | | 208778_s_at, 209464_at, 209642_at, | | | CCT4, CCT5, CCT6A, CCT6B, |
| | | | | | | 210567_s_at, 211036_x_at, 211080_s_at, | | | CCT7, CCT8, CDC14A, CDC16, |
| | | | | | | 211804_s_at, 213226_at, 215509_s_at, | | | CDC20, CDC23, CDC25A, |
| | | | | | | 218350_s_at, 218875_s_at, 221436_s_at | | | CDC26, CDC27, CDC6, CDCA3, |
| | | | | | | | | | CDK1, CDK2, CKS1B, FBXO5, |
| | | | | | | | | | FZR1, GMNN, KIF22, MAD2L1, |
| | | | | | | | | | MAD2L2, NEK2, ORC1, PLK1, |
| | | | | | | | | | PRKACA, PRKACB, PRKACG, |
| | | | | | | | | | PTTG1, RASSF1, SKP2, TCP1 |
| Cell cycle_The | 2.83E−10 | 16 | 16 | AURKA, AURKB, BUB1, | 23 | 200037_s_at, 201091_s_at, 203362_s_at, | 31 | 36 | AURKA, AURKB, AURKC, |
| metaphase checkpoint | | | | BUB1B, CBX3, CBX5, | | 203755_at, 204026_s_at, 204092_s_at, | | | BIRC5, BUB1, BUB1B, BUB3, |
| | | | | CENPA, CENPF, KNTC1, | | 204162_at, 4 204641_at, 204962_s_at, | | | CASC5, CBX3, CBX5, CDC20, |
| | | | | MAD2L1, MIS12, NDC80, | | 206316_s_at, 208079_s_at, 208080_at, | | | CENPA, CENPB, CENPC1, |
| | | | | NEK2, NSL1, ZWILCH, ZWINT | | 209172_s_at, 209464_at, 209484_s_at, | | | CENPE, CENPF, CENPH, |
| | | | | | | 209642_at, 209715_at, 210821_x_at, | | | DSN1, DYNC1H1, INCENP, |
| | | | | | | 211080_s_at, 212126_at, 215509_s_at, | | | KNTC1, MAD1L1, MAD2L1, |
| | | | | | | 218349_s_at, 221559_s_at | | | MAD2L2, MIS12, NDC80, NEK2, |
| | | | | | | | | | NSL1, NUF2, PLK1, PMF1, |
| | | | | | | | | | SPC24, SPC25, ZW10, |
| | | | | | | | | | ZWILCH, ZWINT |
| Cell cycle_Start of | 8.42E−08 | 12 | 18 | CBX5, CDC6, CDC7, CDK2, | 22 | 201528_at, 201555_at, 201930_at, | 24 | 43 | CBX5, CCNE1, CDC45, CDC6, |
| DNA replication in | | | | DBF4, GMNN, H1FX, MCM10, | | 202107_s_at, 203351_s_at, 203352_at, | | | CDC7, CDK2, CDT1, DBF4, |
| early S phase | | | | MCM2, MCM3, MCM6, MCM7, | | 203968_s_at, 204244_s_at, 204252_at, | | | DBF4B, E2F1, GMNN, H1F0, |
| | | | | ORC2, ORC4, POLA2, PRIM1, | | 204441_s_at, 204510_at, 204805_s_at, | | | H1FOO, H1FX, HIST1H1A, |
| | | | | PRIM2, RPA1 | | 204853_at, 205053_at, 208795_s_at, | | | HIST1H1B, HIST1H1C, |
| | | | | | | 209715_at, 210983_s_at, 211804_s_at, | | | HIST1H1D, HIST1H1E, |
| | | | | | | 212126_at, 215708_s_at, 218350_s_at, | | | HIST1H1T, MCM10, MCM2, |
| | | | | | | 220651_s_at | | | MCM3, MCM4, MCM5, MCM6, |
| | | | | | | | | | MCM7, ORC1, ORC2, ORC3, |
| | | | | | | | | | ORC4, ORC5, ORC6, POLA1, |
| | | | | | | | | | POLA2, PPP2CA, PPP2CB, |
| | | | | | | | | | PRIM1, PRIM2, RPA1, RPA2, |
| | | | | | | | | | RPA3, TFDP1 |
| Cell cycle_Spindle | 5.75E−07 | 10 | 18 | ANAPC5, AURKA, AURKB, | 29 | 200098_s_at, 200703_at, 200750_s_at, | 19 | 94 | ACTB, ACTR10, ACTR1A, |
| assembly and | | | | CSE1L, DCTN2, DYNLL1, | | 200932_s_at, 201090_x_at, 201111_at, | | | ACTR1B, ANAPC1, ANAPC10, |
| chromosome | | | | ESPL1, KPNB1, MAD2L1, | | 201112_s_at, 202293_at, 203362_s_at, | | | ANAPC11, ANAPC13, ANAPC2, |
| separation | | | | NDC80, NEK2, RAN, STAG1, | | 204092_s_at, 204162_at, 204641_at, | | | ANAPC4, ANAPC5, ANAPC7, |
| | | | | TPX2, TUBA1B, TUBA3C, | | 204817_at, 208079_s_at, 208080_at, | | | AURKA, AURKB, CAPZA1, |
| | | | | TUBB, TUBB2B | | 208721_s_at, 208722_s_at, 208975_s_at, | | | CAPZA2, CAPZA3, CAPZB, |
| | | | | | | 209026_x_at, 209464_at, 210052_s_at, | | | CCNB1, CCNB2, CCNB3, |
| | | | | | | 210527_x_at, 210766_s_at, 211036_x_at, | | | CDC16, CDC20, CDC23, |
| | | | | | | 211080_s_at, 211714_x_at, 213646_x_at, | | | CDC26, CDC27, CDK1, CSE1L, |
| | | | | | | 214023_x_at, 38158_at | | | DCTN1, DCTN2, DCTN3, |
| | | | | | | | | | DCTN4, DCTN5, DCTN6, |
| | | | | | | | | | DYNC1H1, DYNC1I1, DYNC1I2, |
| | | | | | | | | | DYNC1LI1, DYNC1LI2, |
| | | | | | | | | | DYNLL1, DYNLL2, DYNLRB1, |
| | | | | | | | | | DYNLRB2, DYNLT1, DYNLT3, |
| | | | | | | | | | ESPL1, IPO5, KIF11, KIF22, |
| | | | | | | | | | KPNA1, KPNA2, KPNA3, |
| | | | | | | | | | KPNA4, KPNA5, KPNA6, |
| | | | | | | | | | KPNB1, MAD1L1, MAD2L1, |
| | | | | | | | | | NDC80, NEK2, NUMA1, PTTG1, |
| | | | | | | | | | RAD21, RAN, RCC1, SMC1A, |
| | | | | | | | | | SMC3, STAG1, STAG2, TNPO1, |
| | | | | | | | | | TPX2, TUBA1A, TUBA1B, |
| | | | | | | | | | TUBA1C, TUBA3C, TUBA3D, |
| | | | | | | | | | TUBA3E, TUBA4A, TUBA4B, |
| | | | | | | | | | TUBA8, TUBAL3, TUBB, |
| | | | | | | | | | TUBB1, TUBB2A, TUBB2B, |
| | | | | | | | | | TUBB3, TUBB4A, TUBB4B, |
| | | | | | | | | | TUBB6, TUBB7P, TUBB8, UBB, |
| | | | | | | | | | UBC, ZW10 |
| DNA | 4.42E−05 | 9 | 10 | BLM, BRCA1, CCNA2, | 14 | 201202_at, 202246_s_at, 203418_at, | 23 | 43 | ATM, ATR, ATRIP, BARD1, |
| damage_ATM/ATR | | | | CDC25A, CDK2, CDK4, | | 204252_at, 204531_s_at, 204695_at, | | | BLM, BRCA1, CCNA1, CCNA2, |
| regulation of G1/S | | | | CHEK1, CHEK2, FANCL, | | 205393_s_at, 205394_at, 205733_at, | | | CCND1, CCND2, CCND3, |
| checkpoint | | | | PCNA | | 210416_s_at, 211804_s_at, 211851_x_at, | | | CCNE1, CDC25A, CDK2, CDK4, |
| | | | | | | 213226_at, 218397_at | | | CDKN1A, CHEK1, CHEK2, |
| | | | | | | | | | CLSPN, FANCD2, FANCL, |
| | | | | | | | | | GADD45A, GADD45B, MDC1, |
| | | | | | | | | | MDM2, MYC, NBN, NFKB1, |
| | | | | | | | | | NFKB2, NFKBIA, NFKBIB, |
| | | | | | | | | | NFKBIE, PCNA, RAD9A, |
| | | | | | | | | | RAD9B, REL, RELA, RELB, |
| | | | | | | | | | SMC1A, TP53, UBB, UBC, |
| | | | | | | | | | USP1 |
| DNA | 9.53E−05 | 6 | 10 | EXO1, MSH2, MSH6, PCNA, | 12 | 201202_at, 201528_at, 202911_at, | 11 | 20 | EXO1, MLH1, MSH2, MSH3, |
| damage_Mismatch | | | | PMS2, POLE, RFC2, RFC4, | | 203209_at, 203210_s_at, 203696_s_at, | | | MSH6, PCNA, PMS1, PMS2, |
| repair | | | | RFC5, RPA1 | | 204023_at, 204603_at, 209421_at, | | | POLE, POLE2, POLE3, POLE4, |
| | | | | | | 209805_at, 211450_s_at, 216026_s_at | | | POLH, RFC2, RFC3, RFC4, |
| | | | | | | | | | RFC5, RPA1, RPA2, RPA3 |
| Cell | 9.53E−05 | 6 | 11 | AKAP8, AURKA, AURKB, | 18 | 200080_s_at, 201292_at, 201774_s_at, | 11 | 33 | AKAP8, AURKA, AURKB, |
| cycle_Chromosome | | | | CCNA2, H1FX, H3F3A, | | 203418_at, 203847_s_at, 204092_s_at, | | | CCNA1, CCNA2, CCNB1, |
| condensation in | | | | NCAPD2, NCAPG, NCAPG2, | | 204805_s_at, 208079_s_at, 208080_at, | | | CCNB2, CCNB3, CDK1, H1F0, |
| prometaphase | | | | NCAPH, TOP2A | | 208755_x_at, 209464_at, 211940_x_at, | | | H1FOO, H1FX, H3F3A, H3F3B, |
| | | | | | | 212949_at, 213226_at, 213828_x_at, | | | HIST1H1A, HIST1H1B, |
| | | | | | | 218662_s_at, 218663_at, 219588_s_at | | | HIST1H1C, HIST1H1D, |
| | | | | | | | | | HIST1H1E, HIST1H1T, |
| | | | | | | | | | HIST3H3, INCENP, NCAPD2, |
| | | | | | | | | | NCAPD3, NCAPG, NCAPG2, |
| | | | | | | | | | NCAPH, NCAPH2, SMC2, |
| | | | | | | | | | SMC4, TOP1, TOP2A, TOP2B |
| Cell cycle_Role of | 9.54E−05 | 9 | 10 | ANAPC5, CDC25A, CDC34, | 16 | 200098_s_at, 201897_s_at, 202246_s_at, | 25 | 42 | ANAPC1, ANAPC10, ANAPC11, |
| SCF complex in cell | | | | CDK2, CDK4, CHEK1, | | 203625_x_at, 204252_at, 204695_at, | | | ANAPC13, ANAPC2, ANAPC4, |
| cycle regulation | | | | CKS1B, CUL1, FBXO5, SKP2 | | 205393_s_at, 205394_at, 207614_s_at, | | | ANAPC5, ANAPC7, BTRC, |
| | | | | | | 208721_s_at, 208722_s_at, 210567_s_at, | | | CCND1, CCNE1, CDC16, |
| | | | | | | 211036_x_at, 211804_s_at, 212540_at, | | | CDC23, CDC25A, CDC26, |
| | | | | | | 218875_s_at | | | CDC27, CDC34, CDK1, CDK2, |
| | | | | | | | | | CDK4, CDKN1A, CDKN1B, |
| | | | | | | | | | CDTI, CHEK1, CKS1B, CUL1, |
| | | | | | | | | | E2F1, FBXO5, FBXW11, |
| | | | | | | | | | FBXW7, FZR1, NEDD8, PLK1, |
| | | | | | | | | | RBL2, RBX1, SKP1, SKP2, |
| | | | | | | | | | SMAD3, UBA1, UBB, UBC, |
| | | | | | | | | | WEE1 |
| Methionine | 1.78E−04 | 6 | 6 | AHCY, CTH, DNMT1, | 8 | 200903_s_at, 201475_x_at, 201697_s_at, | 12 | 15 | AHCY, AHCYL1, AHCYL2, |
| metabolism | | | | DNMT3A, MARS, MAT1A | | 205813_s_at, 213671_s_at, 213672_at, | | | BHMT, BHMT2, CBS, CTH, |
| | | | | | | 217127_at, 218457_s_at | | | DNMT1, DNMT3A, DNMT3B, |
| | | | | | | | | | MARS, MAT1A, MAT2A, |
| | | | | | | | | | MTFMT, MTR |
| Apoptosis and | 3.07E−04 | 6 | 6 | BLM, BRCA1, CHEK1, | 9 | 204531_s_at, 205393_s_at, 205394_at, | 13 | 16 | ABL1, ATM, ATR, BLM, BRCA1, |
| survival_DNA- | | | | CHEK2, FANCL, PRKDC | | 205733_at, 208694_at, 210416_s_at, | | | CHEK1, CHEK2, E2F1, |
| damage-induced | | | | | | 210543_s_at, 211851_x_at, 218397_at | | | FANCD2, FANCL, H2AFX, NBN, |
| apoptosis | | | | | | | | | PRKDC, RAD9A, RAD9B, TP53 |
| DNA damage_Role of | 5.94E−04 | 8 | 10 | BRCA1, CHEK2, FANCL, | 12 | 201202_at, 202911_at, 203616_at, | 25 | 40 | ATF1, ATM, ATR, BARD1, |
| Brca1 and Brca2 in | | | | MSH2, MSH6, PCNA, POLB, | | 204531_s_at, 205024_s_at, 209421_at, | | | BRCA1, BRCA2, BRIP1, |
| DNA repair | | | | POLR2D, POLR2J, RAD51 | | 210416_s_at, 211450_s_at, 211851_x_at, | | | CHEK2, DDB2, FANCD2, |
| | | | | | | 212782_x_at, 214144_at, 218397_at | | | FANCL, H2AFX, MDC1, MLH1, |
| | | | | | | | | | MRE11A, MSH2, MSH3, MSH6, |
| | | | | | | | | | NBN, NTHL1, PCNA, POLB, |
| | | | | | | | | | POLR2A, POLR2B, POLR2C, |
| | | | | | | | | | POLR2D, POLR2E, POLR2F, |
| | | | | | | | | | POLR2G, POLR2H, POLR2I, |
| | | | | | | | | | POLR2J, POLR2J2, POLR2K, |
| | | | | | | | | | POLR2L, RAD50, RAD51, TP53, |
| | | | | | | | | | TP53BP1, XPC |

Overall, within the significantly enriched biological pathways, a total of 238 probesets (corresponding to 162 unique genes) were significantly correlated with let-7b (FIG. 9C, Tables 6 and 7). Subsequently, for each of the 162 genes, we selected a representative probeset that exhibits the highest correlation with let-7b and performed DDg analysis (FIG. 9D). Our results revealed that of the 162 genes, 103 genes (63.5%) could significantly and independently stratify patients into low and high-risk subgroups, based on post-surgery OS (P-value <0.05). Next, from the list of 103 survival significant genes, we identified a survival prognostic signature (SPS) comprising the top 36 survival significant genes, which was able to discriminate patients into three distinct subgroups with relatively low-, intermediate- and high-risk outcomes (P-value=1.27E-19, FIG. 9D, Table 8).

TABLE 8
Compositions and associated pathways of 36 genes generated from statistical-
weighted voting procedure. SWVg gave 106 patients in the low-risk group, 188 in the
intermediate-risk group, and 56 in the high-risk group. The log-rank p-value from the SWVg
procedure was 1.27E−19.

| | | | Targets of let-7b | | 1 DDg |
|---|---|---|---|---|---|
| Probeset | Gene | Gene name | based on literature | Involvement in pathways | P-value |
| 205382_s_at | CFD | complement factor D | | Immune response_Alternative complement | 3.17E−04 |
| | | (adipsin) | | pathway | |
| 204451_at | FZD1 | frizzled homolog 1 | | Development_Regulation of epithelial-to- | 5.96E−04 |
| | | (Drosophila) | | mesenchymal transition (EMT) | |
| 202246_s_at | CDK4 | cyclin-dependent | | DNA damage_ATM/ATR regulation of G1/S | 6.64E−04 |
| | | kinase 4 | | checkpoint\|Cell cycle_Role of SCF complex in | |
| | | | | cell cycle regulation | |
| 201947_s_at | CCT2 | chaperonin | Predicted | Cell cycle_Role of APC in cell cycle | 8.42E−04 |
| | | containing TCP1, | | regulation | |
| | | subunit 2 (beta) | | | |
| 205959_at | MMP13 | matrix | | Cell adhesion_ECM remodeling\|Cell | 9.02E−04 |
| | | metallopeptidase 13 | | adhesion_Chemokines and adhesion | |
| | | (collagenase 3) | | | |
| 201615_x_at | CALD1 | caldesmon 1 | Predicted\|TargetScan | Development_Regulation of epithelial-to- | 1.24E−03 |
| | | | | mesenchymal transition (EMT) | |
| 201954_at | ARPC1B | actin related protein | Predicted | Cell adhesion_Chemokines and adhesion | 1.65E−03 |
| | | 2/3 complex, subunit | | | |
| | | 1B, 41 kDa | | | |
| 204464_s_at | EDNRA | endothelin receptor | | Development_Regulation of epithelial-to- | 1.85E−03 |
| | | type A | | mesenchymal transition (EMT) | |
| 203968_s_at | CDC6 | cell division cycle 6 | | Cell cycle_Role of APC in cell cycle | 1.89E−03 |
| | | homolog (S. cerevisiae) | | regulation\|Cell cycle_Start of DNA replication | |
| | | | | in early S phase | |
| 209026_x_at | TUBB | tubulin, beta | Predicted\|TargetScan | Cell cycle_Spindle assembly and chromosome | 2.03E−03 |
| | | | | separation | |
| 201774_s_at | NCAPD2 | non-SMC condensin | | Cell cycle_Chromosome condensation in | 2.17E−03 |
| | | I complex, subunit | | prometaphase | |
| | | D2 | | | |
| 208944_at | TGFBR2 | transforming growth | | Development_Regulation of epithelial-to- | 2.47E−03 |
| | | factor, beta receptor | | mesenchymal transition (EMT) | |
| | | II (70/80 kDa) | | | |
| 212063_at | CD44 | CD44 molecule | | Cell adhesion_ECM remodeling\|Cell | 2.79E−03 |
| | | (Indian blood group) | | adhesion_Chemokines and adhesion | |
| 214144_at | POLR2D | polymerase (RNA) II | Predicted\|TargetScan | DNA damage_Role of Brca1 and Brca2 in | 2.88E−03 |
| | | (DNA directed) | | DNA repair | |
| | | polypeptide D | | | |
| 212239_at | PIK3R1 | phosphoinositide-3- | | Cell adhesion_Chemokines and adhesion | 3.23E−03 |
| | | kinase, regulatory | | | |
| | | subunit 1 (alpha) | | | |
| 203131_at | PDGFRA | platelet-derived | Validated | Development_Regulation of epithelial-to- | 3.41E−03 |
| | | growth factor | | mesenchymal transition (EMT) | |
| | | receptor, alpha | | | |
| | | polypeptide | | | |
| 212782_x_at | POLR2J | polymerase (RNA) II | | DNA damage_Role of Brca1 and Brca2 in | 3.48E−03 |
| | | (DNA directed) | | DNA repair | |
| | | polypeptide J, | | | |
| | | 13.3 kDa | | | |
| 207822_at | FGFR1 | fibroblast growth | TargetScan | Development_Regulation of epithelial-to- | 3.50E−03 |
| | | factor receptor 1 | | mesenchymal transition (EMT) | |
| 209960_at | HGF | hepatocyte growth | Predicted\|TargetScan | Development_Regulation of epithelial-to- | 4.18E−03 |
| | | factor (hepapoietin A; | | mesenchymal transition (EMT) | |
| | | scatter factor) | | | |
| 212294_at | GNG12 | guanine nucleotide | | Cell adhesion_Chemokines and adhesion | 4.51E−03 |
| | | binding protein (G | | | |
| | | protein), gamma 12 | | | |
| 219588_s_at | NCAPG2 | non-SMC condensin | Validated | Cell cycle_Chromosome condensation in | 4.77E−03 |
| | | II complex, subunit | | prometaphase | |
| | | G2 | | | |
| 216598_s_at | CCL2 | chemokine (C-C | | Cell adhesion_Chemokines and adhesion | 4.92E−03 |
| | | motif) ligand 2 | | | |
| 204441_s_at | POLA2 | polymerase (DNA | | Cell cycle_Start of DNA replication in early S | 6.12E−03 |
| | | directed), alpha 2 | | phase | |
| | | (70 kD subunit) | | | |
| 210845_s_at | PLAUR | plasminogen | Predicted | Cell adhesion_ECM remodeling\|Cell | 7.17E−03 |
| | | activator, urokinase | | adhesion_Chemokines and adhesion | |
| | | receptor | | | |
| 202202_s_at | LAMA4 | laminin, alpha 4 | | Cell adhesion_ECM remodeling\|Cell | 7.21E−03 |
| | | | | adhesion_Chemokines and adhesion | |
| 201697_s_at | DNMT1 | DNA (cytosine-5-)- | | Methionine metabolism | 7.45E−03 |
| | | methyltransferase 1 | | | |
| 202107_s_at | MCM2 | minichromosome | | Cell cycle_Start of DNA replication in early S | 7.57E−03 |
| | | maintenance | | phase | |
| | | complex component 2 | | | |
| 215076_s_at | COL3A1 | collagen, type III, | Predicted\|TargetScan | Cell adhesion_ECM remodeling | 8.57E−03 |
| | | alpha 1 | | | |
| 208778_s_at | TCP1 | t-complex 1 | | Cell cycle_Role of APC in cell cycle | 9.41E−03 |
| | | | | regulation | |
| 200931_s_at | VCL | vinculin | Predicted\|TargetScan | Cell adhesion_Chemokines and adhesion | 9.47E−03 |
| 212949_at | NCAPH | non-SMC condensin | | Cell cycle_Chromosome condensation in | 1.01E−02 |
| | | I complex, subunit H | | prometaphase | |
| 201091_s_at | CBX3 | chromobox homolog 3 | | Cell cycle_The metaphase checkpoint | 1.04E−02 |
| 205393_s_at | CHEK1 | CHK1 checkpoint | Predicted | DNA damage_ATM/ATR regulation of G1/S | 1.12E−02 |
| | | homolog (S. pombe) | | checkpoint\|Cell cycle_Role of SCF complex in | |
| | | | | cell cycle regulation\|Apoptosis and | |
| | | | | survival_DNA-damage-induced apoptosis | |
| 203323_at | CAV2 | caveolin 2 | | Cell adhesion_Chemokines and adhesion | 1.16E−02 |
| 202877_s_at | CD93 | CD93 molecule | | Immune response_Classical complement | 1.19E−02 |
| | | | | pathway\|Immune response_Lectin induced | |
| | | | | complement pathway | |
| 221559_s_at | MIS12 | MIS12, MIND | | Cell cycle_The metaphase checkpoint | 1.21E−02 |
| | | kinetochore complex | | | |
| | | component, homolog | | | |
| | | (S. pombe) | | | |

The majority of the SPS genes could be considered as novel prospective biomarkers, with only six SPS genes (PDGFRA, CDK4, CCL2, DNMT1, LAMA4 and GNG12) previously known to be in an OC signature.

Importantly, the 5-year OS rates for the low- and high-risk subgroups by our SPS signature were 64% and 10%, respectively. The univariate analysis showed that the hazard ratio (HR) of high-risk with respect to low-risk was 7.78, with a confidence interval (CI) of 4.84 to 12.52 (P-value <1E-16, Table 9).

TABLE 9
A Univariate Cox proportional hazard analysis of
factors associated with overall survival rates

| Characteristics | | | HR | 95% CI | p-value |
|---|---|---|---|---|---|
| 2 groups | DDg groups | low risk group | 1 | | |
| | (9 let-7s) | high and intermediate risk | 1.71 | 1.33-2.20 | 2.34E−05 |
| | | groups | | | |
| | DDg groups | high risk group | 1 | | |
| | (9 let-7s) | good and intermediate risk | 0.42 | 0.29-0.64 | 4.19E−05 |
| | | groups | | | |
| | DDg groups | low risk group | 1 | | |
| | (36 mRNAs) | high and intermediate risk | 4.55 | 3.10-6.67 | 8.99E−15 |
| | | groups | | | |
| | DDg groups | high risk group | 1 | | |
| | (36 mRNAs) | good and intermediate risk | 0.34 | 0.24-0.48 | 2.16E−09 |
| | | groups | | | |
| | Tumor stage | low (stage I, II) | 1 | | |
| | | high (stage III, IV) | 3.26 | 1.34-7.92 | 0.0092 |
| | Tumor grade | low (grade 1, 2) | 1 | | |
| | | high (grade 3, 4) | 1.52 | 1.01-2.27 | 0.043 |
| | Tumor residual disease | No Macroscopic disease | 1 | | |
| | | >1 mm | 1.98 | 1.23-3.20 | 0.0048 |
| | Venous invasion | No | 1 | | |
| | | Yes | 0.55 | 0.29-1.07 | 0.07682 |
| | Primary therapy | complete response | 1 | | |
| | outcome success | partial response, progressive | 3.3 | 2.36-4.61 | 2.47E−12 |
| | | disease and stable disease | | | |
| 3 groups | DDg groups | low risk group | 1 | | |
| | (9 let-7) | intermediate risk group | 1.58 | 1.22-2.05 | 0.00056 |
| | | high risk group | 2.93 | 1.91-4.50 | 9.32E−07 |
| | DDg groups | low risk group | 1 | | |
| | (36 mRNAs) | intermediate risk group | 4.06 | 2.74-6.02 | 2.93E−12 |
| | | high risk group | 7.78 | 4.84-12.52 | <1E−16 |
| | Tumor residual disease | >20 mm | 1 | | |
| | | 1-20 mm | 1.05 | 0.73-1.51 | 0.78 |
| | | No Macroscopic disease | 0.52 | 0.30-0.91 | 0.021 |
| | Age | age <= 52 | 1 | | |
| | | 53 <= age <= 66 | 1.2 | 0.81-1.78 | 0.36 |
| | | age >= 67 | 1.71 | 1.12-2.61 | 0.012 |
| | Primary therapy | complete response | 1 | | |
| | outcome success | partial response | 3.7 | 2.49-5.51 | 1.21E−10 |
| | | progressive disease and stable | 2.92 | 1.91-4.45 | 6.63E−07 |
| | | disease | | | |

In Table 9, patients belonging to the TCGA ovarian cancer dataset were analyzed. P-values were obtained from the Wald statistic. Only significant factors are included here.

Multivariate and survival analyses indicated that SPS could provide a strong post-surgery prognostic classification of patients that surpasses clinicopathological parameters, such as histological grade/stage, or conventional biomarkers, such as CA125, HE4, P53, or MYC (Table 10, FIG. 11A-11J).

TABLE 10
Multivariate Cox proportional hazard analysis
of factors associated with overall survival rates

| characteristics | | | HR | 95% CI | p-value |
|---|---|---|---|---|---|
| DDg | DDg groups | low risk subgroup | 1 | | |
| groups (9 | | intermediate risk subgroup | 0.37 | 0.15-0.91 | 0.030 |
| let-7s) with | | high risk subgroup | 0.18 | 0.02-1.58 | 0.12 |
| other | Tumor stage | low (stage I, II) | 1 | | |
| clinical | | high (stage III, IV) | 2.47 | 0.44-13.94 | 0.30 |
| indicators | Tumor grade | low (grade 1, 2) | 1 | | |
| | | high (grade 3, 4) | 0.95 | 0.26-3.43 | 0.93 |
| | Tumor residual | No Macroscopic disease | 1 | | |
| | disease | 1-10 mm | 1.57 | 0.59-4.20 | 0.36 |
| | | 11-20 mm | 4.45 | 0.98-20.29 | 0.054 |
| | | >20 mm | 3.22 | 0.94-11.00 | 0.062 |
| | Age | age <= 52 | 1 | | |
| | | 53 <= age <= 66 | 1.22 | 0.49-3.04 | 0.67 |
| | | age >= 67 | 1.27 | 0.45-3.63 | 0.65 |
| | Race | White | 1 | | |
| | | others | 5.48 | 1.49-20.12 | 0.010 |
| | Venous invasion | No | 1 | | |
| | | Yes | 0.15 | 0.03-0.72 | 0.018 |
| | Lymphatic | No | 1 | | |
| | invasion | yes | 2.76 | 0.57-13.42 | 0.21 |
| DDg | DDg groups | low risk subgroup | 1 | | |
| groups (36 | | intermediate risk subgroup | 2.85 | 1.06-7.67 | 0.038 |
| mRNAs) | | high risk subgroup | 28.12 | 5.21-151.85 | 1.05E−04 |
| with other | Tumor stage | low (stage I, II) | 1 | | |
| clinical | | high (stage III, IV) | 1.84 | 0.34-10.08 | 0.48 |
| indicators | Tumor grade | low (grade 1, 2) | 1 | | |
| | | high (grade 3, 4) | 1.47 | 0.39-5.57 | 0.57 |
| | Tumor residual | No Macroscopic disease | 1 | | |
| | disease | 1-10 mm | 0.94 | 0.34-2.59 | 0.91 |
| | | 11-20 mm | 3.66 | 0.82-16.28 | 0.088 |
| | | >20 mm | 1.25 | 0.35-4.46 | 0.73 |
| | Age | age <= 52 | 1 | | |
| | | 53 <= age <= 66 | 1.13 | 0.44-2.89 | 0.80 |
| | | age >= 67 | 0.92 | 0.29-2.89 | 0.89 |
| | Race | White | 1 | | |
| | | others | 5.42 | 1.46-20.12 | 0.011 |
| | Venous invasion | No | 1 | | |
| | | Yes | 0.17 | 0.03-0.91 | 0.038 |
| | Lymphatic | No | 1 | | |
| | invasion | yes | 2.78 | 0.52-14.84 | 0.23 |

### Example 3

### Validation of Prognostic Biomarker Selection and SPS

To validate our procedures of biomarker selection and the computational algorithms used, we randomly generated 999 probeset lists, each containing 162 probesets from a list of negative control probesets and performed similar DDg and SWVg analyses as described earlier. Within, the same TCGA dataset, our SPS significantly outperformed those of the negative controls (FDR=3E-3, FIG. 12).

Next, we validated our SPS and prediction model on three independent datasets—GSE9899, GSE26712, and GSE13876—which contain 246 OC samples (90% in stage III/IV), 185 late-stage HG-OC samples and 157 advanced-stage SOC samples, respectively (FIG. 13). Using the prediction model constructed from TCGA dataset and the 36 SPS genes, each cohorts could be separated into three distinct risk subgroups with log-rank P-value=2.54E-17, 6.54E-11, and 4.62E-8 respectively (FIG. 13A-13C). The low-risk subgroup had a 3-year survival rate of 68-85%, while the intermediate- and high-risk subgroups had 3-year survival rates of 35-57% and 7.7-21%, respectively (Table 11).

TABLE 11
Three-year and five-year survival rated of risk groups in four datasets.

| | | | Patient | | | | |
|---|---|---|---|---|---|---|---|
| | | Number | percentage | 3-year | | 5-year | |
| | | of | within | survival | | survival | |
| Groups | Cohorts | patients | cohorts | rates | 95% CI | rates | 95% CI |
| Low-risk | TCGA | 106 | 30% | 86% | 78%-94% | 64% | 53%-76% |
| subgroup | GSE9899 | 79 | 34% | 85% | 76%-95% | 71% | 56%-88% |
| | GSE26712 | 58 | 45% | 80% | 70%-91% | 64% | 51%-79% |
| | GSE13876 | 41 | 26% | 68% | 54%-85% | 56% | 42%-75% |
| Intermed | TCGA | 188 | 54% | 52% | 44%-61% | 12% | 7.3%-21% |
| late-risk | GSE9899 | 130 | 57% | 57% | 49%-68% | 29% | 19%-43% |
| subgroup | GSE26712 | 59 | 45% | 39% | 28%-54% | 21% | 12%-37% |
| | GSE13876 | 90 | 57% | 35% | 26%-47% | 23% | 15%-34% |
| High-risk | TCGA | 56 | 16% | 21% | 12%-39% | 10% | 3.5%-26% |
| subgroup | GSE9899 | 21 | 9% | 8.4% | 1.5%-48% | 0.0% | 0% |
| | GSE26712 | 13 | 10% | 7.7% | 1.2%-51% | 0.0% | 0% |
| | GSE13876 | 26 | 17% | 14.0% | 5.1%-38% | 4.6% | 0.7%-31% |

Note:
The three subgroups from three evaluation datasets (GSE9899, GSE26712 and GSE13876) were predicted by using the prediction model generated from The Cancer Genome Atlas (TCGA) dataset (same gene design and weight).

The 5-year survival rates were 56-71%, 21-29%, and 0-4.6% for three risk subgroups, respectively. This analysis strongly supports our SPS and suggests the potential application of SPS in clinical settings.

### Example 4

### Comparison of Our Patient Subgrouping with Other Clinically or Molecularly Relevant Groupings

Kappa correlation coefficient revealed significant associations between patient subgroupings based on our risk classification and clinical parameters, such as tumor stage (P-value=3E-4), tumor residual size (P-value=0.01), and chemotherapy response (P-value=1E-3). These findings suggest the potential application of our SPS in predicting therapy outcome (Table 12).

TABLE 12
Association between the overall survival profile with clinico-pathologic characteristics or molecular subtypes.

| | | Low Risk | | Intermediate | | High Risk | | Weighted Kappa | |
|---|---|---|---|---|---|---|---|---|---|
| | | (n = 106) | | Risk (n = 188) | | (n = 56) | | Kappa | |
| Characteristic | Subcategory | Number | % | Number | % | Number | % | coefficient | p-value |
| Age at initial | age ≦ 52 | 37 | 34.91 | 47 | 25.00 | 12 | 21.43 | 0.09875 | 6.201E−02 |
| pathological | 53 ≦ age ≦ 66 | 46 | 43.40 | 76 | 40.43 | 29 | 51.79 | | |
| diagnosis | age ≧ 67 | 23 | 21.70 | 64 | 34.04 | 15 | 26.79 | | |
| | ^{*}others/no information | | | 1 | 0.53 | | | | |
| Stage | Stage I-II | 13 | 12.26 | 10 | 5.32 | 1 | 1.79 | 0.1716 | 2.716E−04 |
| | Stage III | 83 | 78.30 | 147 | 78.19 | 40 | 71.43 | | |
| | Stage IV | 10 | 9.43 | 30 | 15.96 | 15 | 26.79 | | |
| | ^{*}others/no information | | | 1 | 0.53 | | | | |
| Grade | Grade 1 | 1 | 0.94 | 1 | 0.53 | 1 | 1.79 | 0.007746 | 6.702E−01 |
| | Grade 2 | 17 | 16.04 | 21 | 11.17 | 7 | 12.50 | | |
| | Grade 3 | 88 | 86.02 | 162 | 86.17 | 45 | 80.36 | | |
| | ^{*}others/no information | | | 4 | 2.13 | 3 | 5.36 | | |
| Tumor | No_Macroscopic_disease | 23 | 21.70 | 31 | 16.49 | 4 | 7.14 | 0.1476 | 1.079E−02 |
| residual | 1-20 mm | 45 | 42.45 | 103 | 54.79 | 30 | 53.57 | | |
| disease | >20_mm | 14 | 13.21 | 34 | 18.09 | 13 | 23.21 | | |
| | ^{*}others/no information | 24 | 22.64 | 20 | 10.64 | 9 | 16.07 | | |
| Primary | Complete response | 75 | 70.75 | 89 | 47.34 | 19 | 33.93 | 0.1795 | 1.025E−03 |
| therapy | Partial response | 6 | 5.66 | 28 | 14.89 | 14 | 25.00 | | |
| outcome | Stable/Progressive | 10 | 9.43 | 29 | 15.43 | 7 | 12.50 | | |
| success | disease | | | | | | | | |
| | ^{*}others/no information | 15 | 14.15 | 42 | 22.34 | 16 | 28.57 | 0.4533 | 1.146E−18 |
| \hat{ }TCGA | Proliferative | 42 | 39.62 | 42 | 22.34 | 1 | 1.79 | | |
| samples by | lmmunoreactive/Differentiated | 56 | 52.83 | 99 | 52.66 | 19 | 33.93 | | |
| molecular | Mesenchymal | 2 | 1.89 | 42 | 22.34 | 33 | 58.93 | | |
| subtypes | ^{*}others/no information | 6 | 5.66 | 5 | 2.66 | 3 | 5.36 | | |
| \hat{ }TCGA | C1 | 69 | 65.09 | 70 | 37.23 | 9 | 16.07 | 0.2557 | 1.349E−06 |
| samples by | C2 | 10 | 9.43 | 62 | 32.98 | 27 | 48.21 | | |
| miRNA | C3 | 21 | 19.81 | 51 | 27.13 | 17 | 30.36 | | |
| clustering | ^{*}others/no information | 6 | 5.66 | 5 | 2.66 | 3 | 5.36 | | |
| ^{#}Classification | Low risk | 51 | 48.11 | 56 | 29.79 | 7 | 12.50 | 0.3344 | 4.640E−11 |
| from 21 | Intermediate risk | 54 | 50.94 | 121 | 64.36 | 33 | 58.93 | | |
| miRNAs | High risk | 1 | 0.94 | 11 | 5.85 | 16 | 28.57 | | |

Note:
Measure of agreement was calculated using weighted kappa and the significance of the agreement was estimated by Mantel-Haenszel (MH) test. Calculations were implemented using StatXact-9 (Computed Weight: Quadratic Difference, Scores: Equally spaced).
^{*}These subcategories were not included in the calculation of Kappa coefficient.
\hat{ }Sample subgroupings were provided by the authors of TCGA paper (TCGA, 2011).
^{#}The 21 miRNAs, correlated with let-7b in the TCGA dataset are assessed for their patient prognostic classification using DDg and SWVg methods.

Also, we compared our patient classification with previously reported subgroupings, where patients were classified based on molecular subtypes such as differentiated-type, immunoreactive-type, mesenchymal-type and proliferative-type (TCGA, 2011). We observed that our low-risk and high-risk patients were significantly correlated with proliferative-type and mesenchymal-type, respectively (P-value=1E-18, Table 12). However, unlike our classification, which significantly stratified patients into three risk subgroups, the subgrouping based on TCGA molecular subtypes did not show prognostic significance (FIG. 11J).

### Example 5

### Selected miRNA and mRNA are Biomarkers Represented by Patho-Biologically Essential Genes Involved in Significant Pathways, that Synergistically Form Classifiers that can Stratify Patients into Different Risk Subgroups

DDG-SWVg was applied to high-grade epithelial ovarian carcinoma (HG-EOC) data from The Cancer Genome Atlas (TCGA) and Australian Ovarian Cancer Study (AOCS) [GEO accession no. GSE27290], where TCGA was used as a training dataset and AOCS as an independent evaluation dataset. For both datasets, data pre-processing was performed, including identification and removal of poor-quality chips, normalization of data across multiple microarray chips and finally batch effect correction as described above. In the TCGA dataset, survival analysis via DDg method of individual members of let-7 family first revealed the clear heterogeneity of let-7 family, where let-7b and let-7c exhibited pro-oncogenic pattern in HG-EOC. Next, expression correlation analysis of individual let-7 members with all mRNAs revealed the distinctly strong correlation pattern of let-7b when compared to the rest of the let-7 members. Pathway enrichment analyses were performed on two lists of genes using MetaCore from GeneGo Inc.: genes positively correlated with let-7b (Kendall-tau measure of correlation, FDR≦0.01) and genes negatively correlated with let-7b (Kendall-tau measure of correlation, FDR≦0.01). Genes that are significantly correlated with let-7b (Kendall-tau measure of correlation, FDR≦0.01) and also involved in the top significant pathway maps (P≦0.001) were extracted. In this example, FIG. 14 illustrates one of the enriched pathway maps related to EMT. The survival significance of each of the extracted genes was evaluated using DDg method. In this example, FIG. 15 illustrates a number of genes where their expressions independently and significantly stratify patients into two subgroup with distinct overall survival risks. Consequently using SWVg method, the top-ranking survival-significant genes were used to generate a final 36-mRNA prognosis signature which can significantly stratify TCGA HG-EOC patients into low-, intermediate- and high-risk subgroups. This analytical approach (i) allows the identification of a key miRNA member within a miRNA family, (ii) reduces potential biomarker space by the selection of genes that are both significantly correlated with the identified key miRNA from (i) and involved in significant pathways, and (iii) selects biologically meaningful and survival significant genes from (ii) that synergistically form a signature or classifier that can stratify patients into different risk subgroups.

### Example 6

### The Let-7b Associated 36-mRNA Prognostic Signature which Includes Transcripts Encoded by Genes Involved in Cell-Adhesion, EMT Pathway, Cell-Cycle, DNA Damage Repair, Immune Response, Methionine Metabolism, can Significantly Classify HG-EOC Patients into Three Molecular Subgroups of Distinct Risk Patterns

The let-7b associated 36 genes are involved in methionine metabolism (DNMT1), immune response (CFD, CD93), cell-adhesion (MMP13, ARPC1B, CD44, PIK3R1, GNG12, CCL2, PLAUR, LAMA4, COL3A1, VCL, CAV2), regulation of epithelial-to-mesenchymal transition (FZD1, CALD1, EDNRA, TGFBR2, PDGFRA, FGFR1, HGF), DNA damage repair (POLR2D, POLR2J, CDK4, CHEK1) and cell-cycle (CCT2, CDC6, TUBB, NCAPD2, NCAPG2, POLA2, MCM2, TCP1, NCAPH, CBX3, MIS12, CDK4, CHEK1). The 36-mRNA prognosis signature can further stratify these patients into three risk subgroups, of which the low-risk subgroup has a relatively good 5-year survival rate of 65%. On the other hand, the intermediate- and high-risk subgroup has a 5-year survival rate of only 20% and 10% respectively. In a test dataset (AOCS), the 36-mRNA prognosis signature could provide similar classification of these independent patients, by using the prediction model constructed from TCGA dataset, into three risk subgroups (p-value=2.54E-17), of which the low-risk subgroup has a relatively good 5-year survival rate of 72%, while the intermediate- and high-risk subgroup has a 5 year survival rate of 35% and 0% respectively. This evaluation analysis could suggest the application of the 36-mRNA prognosis signature in potential clinical settings.

### Example 7

### The Let-7b Associated 21-miRNA Prognostic Signature

The twenty-one miRNAs (miR-107, miR-103, miR-106b, miR-18a, miR-17-5p, miR-20b, miR-183, miR-25, miR-324-5p, miR-517c, miR-200a, miR-429, miR-200b, miR-96, miR-362, miR-127, miR-214, miR-136, miR-22, miR-320 and miR-486) showed strong correlations with all of the let-7 family members, with fourteen of them negatively correlated with let-7b and let-7c, while seven were positively correlated. Both positively and negatively correlated miRNAs contain known oncogene and tumor suppressors. Using DDg and SWVg, it was observed that TOGA HG-EOC patients can be significantly stratify patients diagnosed with HG-EOC into low-, intermediate- and high-risk subgroups, where the 5-year survival rate is 8%, 22% and 53% respectively (p-value=1E-12). This suggests the application of this 21-miRNA signature in potential clinical settings.

### Example 8

Differential expression and gene ontology analysis of the patient subgroups suggest that 26 key genes involved in HG-SOC regulatory programs could be candidate therapeutic targets.

The results of the differential expression analysis revealed a clear dichotomy of gene function enrichments associated with either transition from lower to higher-risk patients or transition from higher to lower-risk patients. Crucially, we observed that gene sets significantly up-regulated (FDR <0.05) in higher-risk patients relative to lower-risk patients were typically enriched in the genes with GO functions related to ECM, response to wounding, cell motion and angiogenesis (Tables 13 to 18), while gene sets significantly up-regulated in lower-risk patients relative to higher-risk patients were enriched in the genes with GO functions including cell cycle, DNA replication, mitosis and DNA repair. Therefore, distinct and specific cellular programs could dominate during transitions between different prognostic risk subgroups as defined by our SPS, and our results suggest that key genes involved in HG-EOC regulatory programs could be candidate therapeutic targets. Specifically, our analysis revealed that 26 of the 36 genes in our SPS were found to be differentially expressed across the three risk subgroups, with pairwise significance as FDR <0.05 (Table 19). The genes include PDGFRA, CAV2, FZD1, EDNRA, MMP13, HGF, PLAUR and COL3A1, which were independently and collectively are strong survival significant, and could be therapeutic targets (FIG. 13D).

Furthermore, results also suggest that within the 36-mRNA prognostic signature, genes associated with regulation of epithelial-to-mesenchymal transition are enriched (Table 20).

TABLE 13
Upregulated in high-with respect to low-risk groups

| | | Fold | |
|---|---|---|---|
| Term | Count | Enrichment | Benjamini |
| GO: 0005576~extracellular region | 476 | 1.58 | 2.28E−30 |
| GO: 0007155~cell adhesion | 241 | 1.99 | 5.16E−28 |
| GO: 0022610~biological adhesion | 241 | 1.99 | 5.16E−28 |
| GO: 0044421~extracellular region part | 313 | 1.77 | 6.85E−28 |
| GO: 0009611~response to wounding | 199 | 2.06 | 4.79E−25 |
| GO: 0005886~plasma membrane | 799 | 1.32 | 1.11E−24 |
| GO: 0031012~extracellular matrix | 140 | 2.30 | 3.57E−24 |
| GO: 0005578~proteinaceous extracellular matrix | 128 | 2.31 | 2.68E−22 |
| GO: 0006954~inflammatory response | 126 | 2.14 | 1.69E−16 |
| GO: 0006952~defense response | 190 | 1.81 | 4.76E−16 |
| GO: 0006955~immune response | 192 | 1.72 | 2.10E−13 |
| GO: 0044459~plasma membrane part | 544 | 1.30 | 1.37E−12 |
| GO: 0001944~vasculature development | 103 | 2.10 | 1.57E−12 |
| GO: 0005615~extracellular space | 208 | 1.60 | 2.16E−12 |
| GO: 0007166~cell surface receptor linked signal transduction | 364 | 1.42 | 4.34E−12 |
| GO: 0001568~blood vessel development | 100 | 2.08 | 5.19E−12 |
| GO: 0032101~regulation of response to external stimulus | 73 | 2.40 | 5.37E−12 |
| GO: 0005509~calcium ion binding | 232 | 1.58 | 8.59E−12 |
| GO: 0051270~regulation of cell motion | 84 | 2.19 | 2.69E−11 |
| GO: 0030334~regulation of cell migration | 76 | 2.24 | 9.08E−11 |
| GO: 0030198~extracellular matrix organization | 52 | 2.67 | 1.90E−10 |
| GO: 0040012~regulation of locomotion | 81 | 2.15 | 2.23E−10 |
| GO: 0048514~blood vessel morphogenesis | 85 | 2.05 | 1.00E−09 |
| GO: 0009986~cell surface | 117 | 1.75 | 3.07E−09 |
| GO: 0043627~response to estrogen stimulus | 51 | 2.53 | 3.63E−09 |
| GO: 0001525~angiogenesis | 64 | 2.26 | 3.94E−09 |
| GO: 0006928~cell motion | 147 | 1.66 | 4.55E−09 |
| GO: 0005201~extracellular matrix structural constituent | 43 | 2.77 | 5.74E−09 |
| GO: 0019838~growth factor binding | 52 | 2.51 | 7.25E−09 |
| GO: 0016337~cell-cell adhesion | 89 | 1.94 | 9.99E−09 |
| GO: 0042060~wound healing | 72 | 2.09 | 1.74E−08 |
| GO: 0050727~regulation of inflammatory response | 39 | 2.80 | 1.97E−08 |
| GO: 0032103~positive regulation of response to external stimulus | 37 | 2.88 | 2.09E−08 |
| GO: 0031589~cell-substrate adhesion | 47 | 2.52 | 2.16E−08 |
| GO: 0042127~regulation of cell proliferation | 222 | 1.47 | 2.16E−08 |
| GO: 0048545~response to steroid hormone stimulus | 75 | 2.01 | 4.34E−08 |
| GO: 0005539~glycosaminoglycan binding | 58 | 2.24 | 6.10E−08 |
| GO: 0001501~skeletal system development | 106 | 1.77 | 6.46E−08 |
| GO: 0051094~positive regulation of developmental process | 98 | 1.81 | 7.12E−08 |
| GO: 0006897~endocytosis | 79 | 1.95 | 7.37E−08 |
| GO: 0010324~membrane invagination | 79 | 1.95 | 7.37E−08 |
| GO: 0001871~pattern binding | 61 | 2.19 | 7.42E−08 |
| GO: 0030247~polysaccharide binding | 61 | 2.19 | 7.42E−08 |
| GO: 0010033~response to organic substance | 204 | 1.46 | 1.84E−07 |
| GO: 0044420~extracellular matrix part | 49 | 2.25 | 2.14E−07 |
| GO: 0030036~actin cytoskeleton organization | 77 | 1.92 | 2.56E−07 |
| GO: 0051272~positive regulation of cell motion | 47 | 2.36 | 2.83E−07 |
| GO: 0030029~actin filament-based process | 81 | 1.88 | 2.87E−07 |
| GO: 0007167~enzyme linked receptor protein signaling pathway | 114 | 1.68 | 3.50E−07 |
| GO: 0031226~intrinsic to plasma membrane | 322 | 1.31 | 5.08E−07 |

TABLE 14
Upregulated in intermediate-with respect to low-risk groups

| Term | Count | Fold Enrichment | Benjamini |
|---|---|---|---|
| GO: 0031012~extracellular matrix | 89 | 4.35 | 2.87E−32 |
| GO: 0005578~proteinaceous extracellular matrix | 85 | 4.56 | 3.68E−32 |
| GO: 0005576~extracellular region | 217 | 2.14 | 1.06E−29 |
| GO: 0044421~extracellular region part | 155 | 2.60 | 4.66E−29 |
| GO: 0022610~biological adhesion | 107 | 2.70 | 9.78E−19 |
| GO: 0007155~cell adhesion | 107 | 2.70 | 9.78E−19 |
| GO: 0044420~extracellular matrix part | 35 | 4.79 | 3.95E−13 |
| GO: 0030198~extracellular matrix organization | 34 | 5.33 | 8.30E−13 |
| GO: 0005201~extracellular matrix structural constituent | 28 | 5.35 | 1.75E−10 |
| GO: 0009611~response to wounding | 77 | 2.44 | 2.35E−10 |
| GO: 0001501~skeletal system development | 55 | 2.80 | 3.48E−09 |
| GO: 0043062~extracellular structure organization | 36 | 3.69 | 8.45E−09 |
| GO: 0005581~collagen | 17 | 6.90 | 1.69E−08 |
| GO: 0005615~extracellular space | 87 | 1.99 | 2.21E−08 |
| GO: 0030247~polysaccharide binding | 34 | 3.63 | 3.51E−08 |
| GO: 0001871~pattern binding | 34 | 3.63 | 3.51E−08 |
| GO: 0005509~calcium ion binding | 96 | 1.94 | 4.34E−08 |
| GO: 0005539~glycosaminoglycan binding | 32 | 3.67 | 5.05E−08 |
| GO: 0030199~collagen fibril organization | 15 | 8.55 | 6.48E−08 |
| GO: 0001944~vasculature development | 45 | 2.80 | 2.32E−07 |
| GO: 0030246~carbohydrate binding | 49 | 2.55 | 3.17E−07 |
| GO: 0019838~growth factor binding | 26 | 3.73 | 1.57E−06 |
| GO: 0005518~collagen binding | 14 | 6.88 | 2.18E−06 |
| GO: 0001568~blood vessel development | 42 | 2.67 | 3.53E−06 |
| GO: 0031589~cell-substrate adhesion | 24 | 3.93 | 5.69E−06 |
| GO: 0005583~fibrillar collagen | 9 | 10.63 | 8.52E−06 |
| GO: 0006928~cell motion | 61 | 2.11 | 1.00E−05 |
| GO: 0048407~platelet-derived growth factor binding | 9 | 11.26 | 1.03E−05 |
| GO: 0005604~basement membrane | 19 | 4.18 | 1.09E−05 |
| GO: 0030323~respiratory tube development | 24 | 3.76 | 1.17E−05 |
| GO: 0007160~cell-matrix adhesion | 22 | 4.02 | 1.27E−05 |
| GO: 0005178~integrin binding | 18 | 4.42 | 2.13E−05 |
| GO: 0030324~lung development | 23 | 3.73 | 2.40E−05 |
| GO: 0060541~respiratory system development | 24 | 3.53 | 3.28E−05 |
| GO: 0007167~enzyme linked receptor protein signaling pathway | 49 | 2.20 | 5.18E−05 |
| GO: 0060348~bone development | 25 | 3.27 | 6.41E−05 |
| GO: 0035295~tube development | 35 | 2.61 | 6.59E−05 |
| GO: 0001503~ossification | 24 | 3.35 | 6.74E−05 |
| GO: 0042060~wound healing | 31 | 2.74 | 9.97E−05 |
| GO: 0008201~heparin binding | 22 | 3.36 | 1.02E−04 |
| GO: 0005886~plasma membrane | 257 | 1.27 | 1.26E−04 |
| GO: 0001525~angiogenesis | 27 | 2.92 | 1.78E−04 |
| GO: 0009986~cell surface | 46 | 2.05 | 1.80E−04 |
| GO: 0048514~blood vessel morphogenesis | 34 | 2.51 | 1.92E−04 |
| GO: 0032101~regulation of response to external stimulus | 28 | 2.81 | 2.08E−04 |
| GO: 0050840~extracellular matrix binding | 11 | 6.31 | 2.17E−04 |
| GO: 0060205~cytoplasmic membrane-bounded vesicle lumen | 14 | 4.13 | 5.86E−04 |
| GO: 0016337~cell-cell adhesion | 35 | 2.33 | 6.65E−04 |
| GO: 0043627~response to estrogen stimulus | 21 | 3.18 | 7.43E−04 |
| GO: 0043588~skin development | 11 | 5.81 | 9.00E−04 |

TABLE 15
Upregulated in high-with respect to intermediate-risk groups

| Term | Count | Fold Enrichment | Benjamini |
|---|---|---|---|
| GO: 0022610~biological adhesion | 171 | 2.49 | 1.23E−28 |
| GO: 0007155~cell adhesion | 171 | 2.49 | 1.23E−28 |
| GO: 0044421~extracellular region part | 218 | 2.10 | 1.77E−27 |
| GO: 0005576~extracellular region | 311 | 1.77 | 2.29E−26 |
| GO: 0031012~extracellular matrix | 103 | 2.89 | 3.06E−23 |
| GO: 0005578~proteinaceous extracellular matrix | 95 | 2.93 | 6.53E−22 |
| GO: 0005886~plasma membrane | 480 | 1.36 | 6.77E−16 |
| GO: 0009611~response to wounding | 117 | 2.13 | 1.05E−12 |
| GO: 0001944~vasculature development | 74 | 2.65 | 1.96E−12 |
| GO: 0001568~blood vessel development | 72 | 2.64 | 4.92E−12 |
| GO: 0005615~extracellular space | 139 | 1.83 | 1.81E−11 |
| GO: 0019838~growth factor binding | 42 | 3.59 | 5.30E−11 |
| GO: 0030198~extracellular matrix organization | 40 | 3.60 | 1.35E−10 |
| GO: 0044420~extracellular matrix part | 41 | 3.23 | 2.58E−10 |
| GO: 0001525~angiogenesis | 49 | 3.04 | 3.28E−10 |
| GO: 0048514~blood vessel morphogenesis | 61 | 2.59 | 9.80E−10 |
| GO: 0030334~regulation of cell migration | 52 | 2.70 | 8.58E−09 |
| GO: 0048545~response to steroid hormone stimulus | 55 | 2.59 | 1.08E−08 |
| GO: 0040012~regulation of locomotion | 55 | 2.56 | 1.58E−08 |
| GO: 0044459~plasma membrane part | 328 | 1.34 | 2.47E−08 |
| GO: 0043627~response to estrogen stimulus | 37 | 3.23 | 2.68E−08 |
| GO: 0051270~regulation of cell motion | 55 | 2.52 | 2.70E−08 |
| GO: 0006955~immune response | 115 | 1.81 | 3.59E−08 |
| GO: 0042060~wound healing | 51 | 2.60 | 3.71E−08 |
| GO: 0005509~calcium ion binding | 141 | 1.70 | 3.78E−08 |
| GO: 0032101~regulation of response to external stimulus | 47 | 2.71 | 3.94E−08 |
| GO: 0005201~extracellular matrix structural constituent | 31 | 3.53 | 9.63E−08 |
| GO: 0001501~skeletal system development | 72 | 2.11 | 1.56E−07 |
| GO: 0030246~carbohydrate binding | 69 | 2.15 | 1.96E−07 |
| GO: 0040017~positive regulation of locomotion | 35 | 3.09 | 2.48E−07 |
| GO: 0005518~collagen binding | 18 | 5.28 | 3.35E−07 |
| GO: 0001871~pattern binding | 42 | 2.67 | 5.38E−07 |
| GO: 0030247~polysaccharide binding | 42 | 2.67 | 5.38E−07 |
| GO: 0005539~glycosaminoglycan binding | 40 | 2.74 | 5.50E−07 |
| GO: 0043062~extracellular structure organization | 44 | 2.60 | 6.50E−07 |
| GO: 0051272~positive regulation of cell motion | 34 | 3.00 | 9.39E−07 |
| GO: 0030335~positive regulation of cell migration | 32 | 3.09 | 1.11E−06 |
| GO: 0030155~regulation of cell adhesion | 40 | 2.69 | 1.13E−06 |
| GO: 0042127~regulation of cell proliferation | 138 | 1.60 | 1.17E−06 |
| GO: 0006952~defense response | 104 | 1.74 | 1.70E−06 |
| GO: 0006928~cell motion | 91 | 1.81 | 1.95E−06 |
| GO: 0009986~cell surface | 74 | 1.89 | 2.22E−06 |
| GO: 0010033~response to organic substance | 128 | 1.61 | 2.95E−06 |
| GO: 0007166~cell surface receptor linked signal transduction | 208 | 1.42 | 2.98E−06 |
| GO: 0009725~response to hormone stimulus | 77 | 1.90 | 3.45E−06 |
| GO: 0009719~response to endogenous stimulus | 83 | 1.84 | 3.56E−06 |
| GO: 0006954~inflammatory response | 67 | 2.00 | 3.68E−06 |
| GO: 0007167~enzyme linked receptor protein signaling pathway | 74 | 1.91 | 4.13E−06 |
| GO: 0016337~cell-cell adhesion | 56 | 2.15 | 4.27E−06 |
| GO: 0005581~collagen | 18 | 4.20 | 4.90E−06 |

TABLE 16
Upregulated in low-with respect to high-risk groups

| | | Fold | |
|---|---|---|---|
| Term | Count | Enrichment | Benjamini |
| GO: 0031981~nuclear lumen | 504 | 2.09 | 3.77E−76 |
| GO: 0070013~intracellular organelle lumen | 574 | 1.94 | 2.91E−74 |
| GO: 0031974~membrane-enclosed lumen | 589 | 1.90 | 1.42E−72 |
| GO: 0043233~organelle lumen | 576 | 1.89 | 3.40E−70 |
| GO: 0005654~nucleoplasm | 346 | 2.28 | 9.51E−61 |
| GO: 0007049~cell cycle | 303 | 2.20 | 1.24E−47 |
| GO: 0000278~mitotic cell cycle | 192 | 2.75 | 4.99E−47 |
| GO: 0005694~chromosome | 196 | 2.71 | 1.35E−46 |
| GO: 0022402~cell cycle process | 244 | 2.40 | 3.67E−46 |
| GO: 0022403~cell cycle phase | 197 | 2.68 | 5.35E−46 |
| GO: 0006259~DNA metabolic process | 216 | 2.48 | 1.98E−43 |
| GO: 0000279~M phase | 162 | 2.87 | 6.21E−43 |
| GO: 0043228~non-membrane-bounded organelle | 613 | 1.56 | 1.72E−40 |
| GO: 0043232~intracellular non-membrane-bounded organelle | 613 | 1.56 | 1.72E−40 |
| GO: 0000087~M phase of mitotic cell cycle | 126 | 3.18 | 1.14E−39 |
| GO: 0007067~mitosis | 124 | 3.20 | 1.78E−39 |
| GO: 0000280~nuclear division | 124 | 3.20 | 1.78E−39 |
| GO: 0048285~organelle fission | 127 | 3.14 | 3.86E−39 |
| GO: 0044427~chromosomal part | 165 | 2.72 | 5.80E−39 |
| GO: 0006396~RNA processing | 219 | 2.32 | 1.69E−38 |
| GO: 0008380~RNA splicing | 143 | 2.84 | 6.07E−37 |
| GO: 0006397~mRNA processing | 150 | 2.65 | 5.74E−34 |
| GO: 0016071~mRNA metabolic process | 165 | 2.51 | 9.02E−34 |
| GO: 0006260~DNA replication | 104 | 3.12 | 2.29E−31 |
| GO: 0000377~RNA splicing, via transesterification reactions | 93 | 3.18 | 8.04E−29 |
| with bulged adenosine as nucleophile | | | |
| GO: 0000375~RNA splicing, via transesterification reactions | 93 | 3.18 | 8.04E−29 |
| GO: 0000398~nuclear mRNA splicing, via spliceosome | 93 | 3.18 | 8.04E−29 |
| GO: 0003677~DNA binding | 508 | 1.54 | 1.11E−28 |
| GO: 0051301~cell division | 130 | 2.55 | 5.43E−27 |
| GO: 0006281~DNA repair | 126 | 2.59 | 5.66E−27 |
| GO: 0003723~RNA binding | 227 | 1.96 | 1.36E−25 |
| GO: 0051276~chromosome organization | 179 | 2.13 | 2.38E−25 |
| GO: 0006974~response to DNA damage stimulus | 151 | 2.28 | 8.96E−25 |
| GO: 0000793~condensed chromosome | 73 | 3.37 | 3.74E−24 |
| GO: 0005730~nucleolus | 217 | 1.91 | 2.18E−23 |
| GO: 0044451~nucleoplasm part | 188 | 2.01 | 3.58E−23 |
| GO: 0000775~chromosome, centromeric region | 65 | 3.41 | 7.40E−22 |
| GO: 0030529~ribonucleoprotein complex | 166 | 2.06 | 1.59E−21 |
| GO: 0005681~spliceosome | 68 | 3.14 | 5.08E−20 |
| GO: 0015630~microtubule cytoskeleton | 167 | 1.99 | 5.93E−20 |
| GO: 0000166~nucleotide binding | 508 | 1.39 | 3.00E−17 |
| GO: 0000785~chromatin | 81 | 2.58 | 8.27E−17 |
| GO: 0006261~DNA-dependent DNA replication | 42 | 3.75 | 2.76E−16 |
| GO: 0000776~kinetochore | 45 | 3.60 | 3.11E−16 |
| GO: 0000779~condensed chromosome, centromeric region | 40 | 3.87 | 3.97E−16 |
| GO: 0007059~chromosome segregation | 49 | 3.35 | 8.02E−16 |
| GO: 0016604~nuclear body | 74 | 2.61 | 1.22E−15 |
| GO: 0033554~cellular response to stress | 180 | 1.79 | 2.15E−15 |
| GO: 0000777~condensed chromosome kinetochore | 37 | 3.97 | 3.66E−15 |
| GO: 0000228~nuclear chromosome | 69 | 2.54 | 1.03E−13 |

TABLE 17
Upregulated in low-with respect to intermediate-risk groups

| | | Fold | |
|---|---|---|---|
| Term | Count | Enrichment | Benjamini |
| GO: 0007049~cell cycle | 151 | 3.40 | 4.50E−41 |
| GO: 0006259~DNA metabolic process | 117 | 4.16 | 4.49E−40 |
| GO: 0022403~cell cycle phase | 106 | 4.46 | 3.83E−39 |
| GO: 0000279~M phase | 92 | 5.06 | 1.54E−38 |
| GO: 0022402~cell cycle process | 121 | 3.69 | 3.40E−36 |
| GO: 0031981~nuclear lumen | 195 | 2.42 | 1.95E−33 |
| GO: 0005694~chromosome | 98 | 4.06 | 1.02E−32 |
| GO: 0000278~mitotic cell cycle | 92 | 4.09 | 2.21E−30 |
| GO: 0000087~M phase of mitotic cell cycle | 69 | 5.40 | 2.54E−30 |
| GO: 0070013~intracellular organelle lumen | 213 | 2.15 | 5.17E−30 |
| GO: 0031974~membrane-enclosed lumen | 219 | 2.11 | 5.87E−30 |
| GO: 0006260~DNA replication | 63 | 5.85 | 7.47E−30 |
| GO: 0000280~nuclear division | 67 | 5.36 | 3.05E−29 |
| GO: 0007067~mitosis | 67 | 5.36 | 3.05E−29 |
| GO: 0048285~organelle fission | 68 | 5.21 | 6.93E−29 |
| GO: 0043228~non-membrane-bounded organelle | 251 | 1.92 | 9.52E−29 |
| GO: 0043232~intracellular non-membrane-bounded organelle | 251 | 1.92 | 9.52E−29 |
| GO: 0043233~organelle lumen | 213 | 2.10 | 1.50E−28 |
| GO: 0005654~nucleoplasm | 134 | 2.64 | 1.19E−25 |
| GO: 0044427~chromosomal part | 79 | 3.90 | 5.08E−25 |
| GO: 0006281~DNA repair | 67 | 4.27 | 9.18E−23 |
| GO: 0051301~cell division | 67 | 4.07 | 1.62E−21 |
| GO: 0006974~response to DNA damage stimulus | 75 | 3.51 | 4.44E−20 |
| GO: 0008380~RNA splicing | 61 | 3.75 | 1.53E−17 |
| GO: 0000377~RNA splicing, via transesterification reactions | 45 | 4.77 | 8.75E−17 |
| with bulged adenosine as nucleophile | | | |
| GO: 0000398~nuclear mRNA splicing, via spliceosome | 45 | 4.77 | 8.75E−17 |
| GO: 0000375~RNA splicing, via transesterification reactions | 45 | 4.77 | 8.75E−17 |
| GO: 0006396~RNA processing | 85 | 2.79 | 2.26E−16 |
| GO: 0000793~condensed chromosome | 38 | 5.26 | 6.21E−16 |
| GO: 0006397~mRNA processing | 62 | 3.40 | 1.25E−15 |
| GO: 0051276~chromosome organization | 77 | 2.84 | 3.90E−15 |
| GO: 0015630~microtubule cytoskeleton | 77 | 2.75 | 9.99E−15 |
| GO: 0000775~chromosome, centromeric region | 34 | 5.35 | 2.26E−14 |
| GO: 0016071~mRNA metabolic process | 65 | 3.07 | 4.04E−14 |
| GO: 0033554~cellular response to stress | 84 | 2.59 | 5.13E−14 |
| GO: 0007059~chromosome segregation | 29 | 6.14 | 1.59E−13 |
| GO: 0006261~DNA-dependent DNA replication | 25 | 6.92 | 7.10E−13 |
| GO: 0005819~spindle | 37 | 4.36 | 1.18E−12 |
| GO: 0005730~nucleolus | 85 | 2.24 | 3.53E−11 |
| GO: 0000226~microtubule cytoskeleton organization | 35 | 4.20 | 5.45E−11 |
| GO: 0007017~microtubule-based process | 46 | 3.35 | 5.50E−11 |
| GO: 0003677~DNA binding | 173 | 1.66 | 4.58E−10 |
| GO: 0000070~mitotic sister chromatid segregation | 18 | 7.62 | 1.14E−09 |
| GO: 0000228~nuclear chromosome | 34 | 3.75 | 1.29E−09 |
| GO: 0000819~sister chromatid segregation | 18 | 7.41 | 2.00E−09 |
| GO: 0007051~spindle organization | 19 | 6.67 | 4.17E−09 |
| GO: 0000776~kinetochore | 22 | 5.27 | 7.09E−09 |
| GO: 0000779~condensed chromosome, centromeric region | 20 | 5.80 | 7.91E−09 |
| GO: 0003723~RNA binding | 80 | 2.18 | 9.12E−09 |
| GO: 0000075~cell cycle checkpoint | 26 | 4.51 | 1.30E−08 |

TABLE 18
Upregulated in intermediate-with respect to high-risk groups

| | | Fold | |
|---|---|---|---|
| Term | Count | Enrichment | Benjamini |
| GO: 0031981~nuclear lumen | 281 | 2.55 | 1.48E−56 |
| GO: 0070013~intracellular organelle lumen | 313 | 2.32 | 1.53E−54 |
| GO: 0043233~organelle lumen | 314 | 2.26 | 2.23E−52 |
| GO: 0031974~membrane-enclosed lumen | 317 | 2.24 | 4.83E−52 |
| GO: 0005654~nucleoplasm | 200 | 2.89 | 8.20E−47 |
| GO: 0022403~cell cycle phase | 127 | 3.79 | 5.84E−40 |
| GO: 0000279~M phase | 109 | 4.24 | 2.06E−39 |
| GO: 0005694~chromosome | 121 | 3.68 | 8.88E−38 |
| GO: 0007049~cell cycle | 174 | 2.78 | 5.97E−36 |
| GO: 0007067~mitosis | 83 | 4.70 | 1.67E−33 |
| GO: 0000280~nuclear division | 83 | 4.70 | 1.67E−33 |
| GO: 0000087~M phase of mitotic cell cycle | 84 | 4.65 | 1.91E−33 |
| GO: 0022402~cell cycle process | 141 | 3.05 | 2.53E−33 |
| GO: 0048285~organelle fission | 84 | 4.55 | 7.84E−33 |
| GO: 0044427~chromosomal part | 101 | 3.65 | 8.40E−31 |
| GO: 0000278~mitotic cell cycle | 109 | 3.43 | 4.15E−30 |
| GO: 0006259~DNA metabolic process | 122 | 3.07 | 6.82E−29 |
| GO: 0043228~non-membrane-bounded organelle | 308 | 1.72 | 5.02E−26 |
| GO: 0043232~intracellular non-membrane-bounded organelle | 308 | 1.72 | 5.02E−26 |
| GO: 0000775~chromosome, centromeric region | 50 | 5.76 | 8.66E−25 |
| GO: 0006396~RNA processing | 120 | 2.79 | 2.90E−24 |
| GO: 0051276~chromosome organization | 111 | 2.90 | 8.73E−24 |
| GO: 0003677~DNA binding | 268 | 1.81 | 2.81E−23 |
| GO: 0008380~RNA splicing | 80 | 3.48 | 4.47E−22 |
| GO: 0051301~cell division | 80 | 3.44 | 1.05E−21 |
| GO: 0006397~mRNA processing | 84 | 3.26 | 4.04E−21 |
| GO: 0006260~DNA replication | 62 | 4.08 | 8.71E−21 |
| GO: 0000793~condensed chromosome | 49 | 4.97 | 9.43E−21 |
| GO: 0003723~RNA binding | 128 | 2.45 | 2.58E−20 |
| GO: 0016071~mRNA metabolic process | 89 | 2.97 | 1.46E−19 |
| GO: 0006974~response to DNA damage stimulus | 88 | 2.91 | 1.12E−18 |
| GO: 0006281~DNA repair | 73 | 3.29 | 1.62E−18 |
| GO: 0044451~nucleoplasm part | 107 | 2.52 | 1.93E−18 |
| GO: 0000377~RNA splicing, via transesterification reactions with | 53 | 3.97 | 5.27E−17 |
| bulged adenosine as nucleophile | | | |
| GO: 0000375~RNA splicing, via transesterification reactions | 53 | 3.97 | 5.27E−17 |
| GO: 0000398~nuclear mRNA splicing, via spliceosome | 53 | 3.97 | 5.27E−17 |
| GO: 0000776~kinetochore | 33 | 5.80 | 4.77E−16 |
| GO: 0007059~chromosome segregation | 35 | 5.25 | 4.07E−15 |
| GO: 0005819~spindle | 46 | 3.98 | 4.22E−15 |
| GO: 0000779~condensed chromosome, centromeric region | 28 | 5.96 | 7.93E−14 |
| GO: 0005730~nucleolus | 111 | 2.15 | 9.63E−14 |
| GO: 0000777~condensed chromosome kinetochore | 26 | 6.12 | 3.61E−13 |
| GO: 0034621~cellular macromolecular complex subunit organization | 74 | 2.61 | 1.34E−12 |
| GO: 0030529~ribonucleoprotein complex | 84 | 2.29 | 1.03E−11 |
| GO: 0016604~nuclear body | 44 | 3.40 | 1.23E−11 |
| GO: 0015630~microtubule cytoskeleton | 84 | 2.20 | 8.25E−11 |
| GO: 0006325~chromatin organization | 71 | 2.45 | 1.26E−10 |
| GO: 0007051~spindle organization | 23 | 5.72 | 2.56E−10 |
| GO: 0051726~regulation of cell cycle | 70 | 2.39 | 5.79E−10 |
| GO: 0000228~nuclear chromosome | 40 | 3.23 | 9.03E−10 |

TABLE 19
Expression levels of signature genes across the SPS-defined risk groups. Differential
expressions were evaluated using a non-parametric Mann-Whitney test. The p-values were
corrected and the false discovery rates (fdr) were calculated using Benjamini-Hochberg step-up method.

| | | | | Log2 | | | |
|---|---|---|---|---|---|---|---|
| | | Log2 | Log2 | fold-change | fdr | | |
| | | fold-change | fold-change | (high-risk/ | (low-risk/ | fdr | fdr |
| | Gene | (intermediate- | (high-risk/ | intermediate- | intermediate- | (low-risk/ | (intermediate- |
| Probe | Symbol | risk/low-risk) | low-risk) | risk) | risk) | high-risk) | risk/high-risk) |
| 200931_s_at | VCL | 1.502E−01 | 3.011E−01 | 1.509E−01 | 8.776E−02 | 9.995E−04 | 3.350E−02 |
| 201091_s_at | CBX3 | −1.422E−01 | −2.976E−01 | −1.554E−01 | 2.626E−02 | 9.430E−04 | 6.903E−02 |
| 201615_x_at | CALD1 | 5.741E−01 | 1.035E+00 | 4.609E−01 | 2.326E−06 | 2.413E−12 | 2.698E−04 |
| 201697_s_at | DNMT1 | −4.000E−01 | −7.317E−01 | −3.317E−01 | 1.179E−05 | 3.473E−09 | 2.154E−03 |
| 201774_s_at | NCAPD2 | −1.624E−01 | −6.141E−01 | −4.516E−01 | 2.437E−01 | 8.303E−06 | 3.955E−04 |
| 201947_s_at | CCT2 | −1.412E−01 | −3.338E−01 | −1.926E−01 | 1.187E−01 | 1.711E−04 | 1.077E−02 |
| 201954_at | ARPC1B | 1.809E−01 | 5.089E−01 | 3.280E−01 | 1.719E−02 | 8.305E−07 | 2.528E−03 |
| 202107_s_at | MCM2 | −3.240E−01 | −8.564E−01 | −5.324E−01 | 6.907E−08 | 1.896E−13 | 5.677E−05 |
| 202202_s_at | LAMA4 | 5.367E−01 | 9.508E−01 | 4.141E−01 | 2.794E−04 | 1.273E−08 | 1.735E−03 |
| 202246_s_at | CDK4 | −2.285E−01 | −5.398E−01 | −3.113E−01 | 9.939E−04 | 5.634E−08 | 2.094E−03 |
| 202877_s_at | CD93 | 1.865E−01 | 5.042E−01 | 3.177E−01 | 6.661E−05 | 1.005E−11 | 4.649E−05 |
| 203131_at | PDGFRA | 7.203E−01 | 1.730E+00 | 1.010E+00 | 4.651E−08 | 3.970E−15 | 6.993E−07 |
| 203323_at | CAV2 | 4.098E−01 | 8.481E−01 | 4.384E−01 | 9.186E−06 | 1.888E−12 | 2.851E−05 |
| 203968_s_at | CDC6 | −1.012E−01 | −2.266E−01 | −1.254E−01 | 6.886E−03 | 3.306E−07 | 2.379E−03 |
| 204441_s_at | POLA2 | −1.701E−01 | −2.658E−01 | −9.575E−02 | 6.891E−05 | 1.198E−07 | 7.325E−03 |
| 204451_at | FZD1 | 4.936E−01 | 1.222E+00 | 7.282E−01 | 3.251E−09 | 6.310E−14 | 2.420E−05 |
| 204464_s_at | EDNRA | 3.870E−01 | 8.869E−01 | 4.998E−01 | 1.330E−05 | 3.801E−10 | 4.138E−04 |
| 205382_s_at | CFD | 2.734E−01 | 7.047E−01 | 4.313E−01 | 2.734E−02 | 9.700E−11 | 4.987E−06 |
| 205393_s_at | CHEK1 | −1.988E−01 | −5.135E−01 | −3.147E−01 | 1.492E−04 | 7.797E−09 | 7.454E−04 |
| 205959_at | MMP13 | 7.030E−02 | 2.681E−01 | 1.978E−01 | 5.311E−04 | 1.967E−10 | 1.567E−04 |
| 207822_at | FGFR1 | 2.130E−01 | 3.198E−01 | 1.068E−01 | 3.842E−02 | 3.060E−03 | 1.894E−01 |
| 208778_s_at | TCP1 | 1.160E−02 | −2.420E−02 | −3.580E−02 | 4.598E−01 | 1.853E−01 | 2.797E−01 |
| 208944_at | TGFBR2 | 4.100E−01 | 8.160E−01 | 4.060E−01 | 4.651E−08 | 2.056E−14 | 7.138E−06 |
| 209026_x_at | TUBB | −1.765E−01 | −5.210E−01 | −3.444E−01 | 3.791E−03 | 3.455E−07 | 1.584E−03 |
| 209960_at | HGF | 6.059E−02 | 1.745E−01 | 1.139E−01 | 4.330E−03 | 1.149E−06 | 4.184E−03 |
| 210845_s_at | PLAUR | 3.496E−01 | 6.870E−01 | 3.375E−01 | 4.185E−03 | 2.690E−08 | 7.092E−04 |
| 212063_at | CD44 | 4.043E−02 | 2.684E−01 | 2.279E−01 | 4.180E−01 | 4.669E−02 | 4.712E−02 |
| 212239_at | PIK3R1 | 2.778E−01 | 4.748E−01 | 1.970E−01 | 1.637E−05 | 1.045E−07 | 3.994E−02 |
| 212294_at | GNG12 | 1.954E−01 | 3.762E−01 | 1.808E−01 | 1.461E−03 | 4.200E−07 | 6.210E−03 |
| 212782_x_at | POLR2J | −7.766E−02 | −1.520E−01 | −7.435E−02 | 1.705E−01 | 2.122E−01 | 4.896E−01 |
| 212949_at | NCAPH | −9.186E−02 | −4.056E−01 | −3.138E−01 | 3.122E−02 | 2.100E−07 | 3.237E−04 |
| 214144_at | POLR2D | −1.162E−01 | −2.103E−01 | −9.415E−02 | 4.013E−03 | 1.141E−06 | 7.424E−03 |
| 215076_s_at | COL3A1 | 1.114E+00 | 1.910E+00 | 7.960E−01 | 1.346E−10 | 1.496E−13 | 2.430E−04 |
| 216598_s_at | CCL2 | 1.730E−01 | 3.726E−01 | 1.996E−01 | 3.505E−01 | 5.121E−02 | 1.179E−01 |
| 219588_s_at | NCAPG2 | −3.039E−01 | −6.294E−01 | −3.255E−01 | 2.878E−04 | 3.121E−10 | 4.185E−04 |
| 221559_s_at | MIS12 | 1.399E−03 | −2.575E−01 | −2.589E−01 | 3.242E−01 | 5.676E−04 | 7.377E−03 |

TABLE 20
Pathway enrichment of genes in the 36-gene signature compared to the background list of 162 genes which are both
significantly correlated with let-7b (FDR < 0.01) and significantly associated with biological pathways (p-value < 0.001).

| | Background = 162 | | | | | |
|---|---|---|---|---|---|---|
| | representative probes | | 36-gene | | Hypergeometric test | |
| | Background | Background | signature | | | fold |
| Significant Pathway (P-value < 0.001) | Count | Ratio | Count | Ratio | P(x >= observed) | enrichment |
| Development_Regulation of epithelial-to-mesenchymal | 19 | 0.117 | 7 | 0.19 | 0.09 | 1.657894737 |
| transition (EMT) | | | | | | |
| Cell adhesion_Chemokines and adhesion | 32 | 0.198 | 10 | 0.28 | 0.13 | 1.40625 |
| Cell cycle_Chromosome condensation in prometaphase | 11 | 0.068 | 3 | 0.08 | 0.46 | 1.227272727 |
| Cell adhesion_ECM remodeling | 22 | 0.136 | 5 | 0.14 | 0.57 | 1.022727273 |
| DNA damage_ATM/ATR regulation of G1/S checkpoint | 10 | 0.062 | 2 | 0.06 | 0.70 | 0.9 |
| Cell cycle_Role of SCF complex in cell cycle regulation | 10 | 0.062 | 2 | 0.06 | 0.70 | 0.9 |
| DNA damage_Role of Brca1 and Brca2 in DNA repair | 10 | 0.062 | 2 | 0.06 | 0.70 | 0.9 |
| Cell cycle_Start of DNA replication in early S phase | 18 | 0.111 | 3 | 0.08 | 0.81 | 0.75 |
| Methionine metabolism | 6 | 0.037 | 1 | 0.03 | 0.78 | 0.75 |
| Apoptosis and survival_DNA-damage-induced apoptosis | 6 | 0.037 | 1 | 0.03 | 0.78 | 0.75 |
| Cell cycle_Role of APC in cell cycle regulation | 19 | 0.117 | 3 | 0.08 | 0.84 | 0.710526316 |
| Cell cycle_The metaphase checkpoint | 16 | 0.099 | 2 | 0.06 | 0.91 | 0.5625 |
| Immune response_Alternative complement pathway | 10 | 0.062 | 1 | 0.03 | 0.93 | 0.45 |
| Immune response_Lectin induced complement pathway | 10 | 0.062 | 1 | 0.03 | 0.93 | 0.45 |
| Immune response_Classical complement pathway | 12 | 0.074 | 1 | 0.03 | 0.96 | 0.375 |
| Cell cycle_Spindle assembly and chromosome | 18 | 0.111 | 1 | 0.03 | 0.99 | 0.25 |
| separation | | | | | | |
| DNA damage_Mismatch repair | 10 | 0.062 | 0 | 0 | 1 | 0 |

Table 20 Pathway enrichment of genes in the 36-gene signature compared to the background list of 162 genes which are both significantly correlated with let-7b (FDR < 0.01) and significantly associated with biological pathways (p-value < 0.001).

### REFERENCES

1. Siegel R, Naishadham D, Jemal A. Cancer statistics, 2012. CA Cancer J Clin 2012; 62:10-29.
2. Cho K R, Shih Ie M. Ovarian cancer. Annu Rev Pathol 2009; 4:287-313.
3. Karst A M, Levanon K, Drapkin R. Modeling high-grade serous ovarian carcinogenesis from the fallopian tube. Proc Natl Acad Sci USA 2011; 108:7547-52.
4. Kim J, Coffey D M, Creighton C J, Yu Z, Hawkins S M, Matzuk M M. High-grade serous ovarian cancer arises from fallopian tube in a mouse model. Proc Natl Acad Sci USA 2012; 109:3921-6.
5. Levanon K, Crum C, Drapkin R. New insights into the pathogenesis of serous ovarian cancer and its clinical impact. J Clin Oncol 2008; 26:5284-93.
6. Shih K K, Qin L X, Tanner E J, Zhou. Q, Bisogna M, Dao F, Olvera N, Viale A, Barakat R R, Levine D A. A microRNA survival signature (MiSS) for advanced ovarian cancer. Gynecol Oncol 2011; 121:444-50.
7. Nam E J, Yoon H, Kim S W, Kim H, Kim Y T, Kim J H, Kim J W, Kim S. MicroRNA expression profiles in serous ovarian carcinoma. Clin Cancer Res 2008; 14:2690-5.
8. Dahiya N, Sherman-Baust C A, Wang T L, Davidson B, Shih le M, Zhang Y, Wood W, 3rd, Becker K G, Morin P J. MicroRNA expression and identification of putative miRNA targets in ovarian cancer. PLoS One 2008; 3:e2436.
9. Zhang L, Volinia S, Bonome T, Calin G A, Greshock J, Yang N, Liu C G, Giannakakis A, Alexiou P, Hasegawa K, Johnstone C N, Megraw M S, et al. Genomic and epigenetic alterations deregulate microRNA expression in human epithelial ovarian cancer. Proc Natl Acad Sci USA 2008; 105:7004-9.
10. Wang Y, Hu X, Greshock J, Shen L, Yang X, Shao Z, Liang S, Tanyi J L, Sood A K, Zhang L. Genomic DNA copy-number alterations of the let-7 family in human cancers. PLoS One 2012; 7:e44399.
11. Vaughan S, Coward J I, Bast R C, Jr., Berchuck A, Berek J S, Brenton J D, Coukos G, Crum C C, Drapkin R, Etemadmoghadam D, Friedlander M, Gabra H, et al. Rethinking ovarian cancer: recommendations for improving outcomes. Nat Rev Cancer 2011; 11:719-25.
12. Tuma R S. Origin of ovarian cancer may have implications for screening. J Natl Cancer Inst 2010; 102:11-3.
13. TCGA. Integrated genomic analyses of ovarian carcinoma. Nature 2011; 474:609-15.
14. Wang V, Li C, Lin M, Welch W, Bell D, Wong Y F, Berkowitz R, Mok S C, Bandera C A. Ovarian cancer is a heterogeneous disease. Cancer Genet Cytogenet 2005; 161:170-3.
15. Helland A, Anglesio M S, George J, Cowin P A, Johnstone C N, House C M, Sheppard K E, Etemadmoghadam D, Melnyk N, Rustgi A K, Phillips W A, Johnsen H, et al. Deregulation of MYCN, LIN28B and LET7 in a molecular subtype of aggressive high-grade serous ovarian cancers. PLoS One 2011; 6:e18064.
16. Calin G A, Croce C M. MicroRNA signatures in human cancers. Nat Rev Cancer 2006; 6:857-66.
17. Chan X H, Nama S, Gopal F, Rizk P, Ramasamy S, Sundaram G, Ow G S, Vladimirovna I A, Tanavde V, Haybaeck J, Kuznetsov V, Sampath P. Targeting Glioma Stem Cells by Functional Inhibition of a Prosurvival OncomiR-138 in Malignant Gliomas. Cell Rep 2012; 2:591-602.
18. Lagos-Quintana M, Rauhut R, Lendeckel W, Tuschl T. Identification of novel genes coding for small expressed RNAs. Science 2001; 294:853-8.
19. Valastyan S, Weinberg R A. Roles for microRNAs in the regulation of cell adhesion molecules. J Cell Sci 2011; 124:999-1006.
20. Reinhart B J, Slack F J, Basson M, Pasquinelli A E, Bettinger J C, Rougvie A E, Horvitz H R, Ruvkun G. The 21-nucleotide let-7 RNA regulates developmental timing in Caenorhabditis elegans. Nature 2000; 403:901-6.
21. Koh W, Sheng C T, Tan B, Lee Q Y, Kuznetsov V, Kiang L S, Tanavde V. Analysis of deep sequencing microRNA expression profile from human embryonic stem cells derived mesenchymal stem cells reveals possible role of let-7 microRNA family in downstream targeting of hepatic nuclear factor 4 alpha. BMC Genomics 2010; 11 Suppl 1:S6.
22. Cancer Genome Atlas Research Network. Comprehensive genomic characterization defines human glioblastoma genes and core pathways. Nature 2008; 455:1061-8.
23. Tothill R W, Tinker A V, George J, Brown R, Fox S B, Lade S, Johnson D S, Trivett M K, Etemadmoghadam D, Locandro B, Traficante N, Fereday S, et al. Novel molecular subtypes of serous and endometrioid ovarian cancer linked to clinical outcome. Clin Cancer Res 2008; 14:5198-208.
24. Bonome T, Levine D A, Shih J, Randonovich M, Pise-Masison C A, Bogomolniy F, Ozbun L, Brady J, Barrett J C, Boyd J, Birrer M J. A gene signature predicting for survival in suboptimally debulked patients with ovarian cancer. Cancer Res 2008; 68:5478-86.
25. Crijns A P, Fehrmann R S, de Jong S, Gerbens F, Meersma G J, Klip H G, Hollema H, Hofstra R M, to Meerman G J, de Vries E G, van der Zee A G. Survival-related profile, pathways, and transcription factors in ovarian cancer. PLoS Med 2009; 6:e24.
26. Hernandez E, Bhagavan B S, Parmley T H, Rosenshein N B. Interobserver variability in the interpretation of epithelial ovarian cancer. Gynecol Oncol 1984; 17:117-23.
27. Johnson W E, Li C, Rabinovic A. Adjusting batch effects in microarray expression data using empirical Bayes methods. Biostatistics 2007; 8:118-27.
28. Kerr M K, Churchill G A. Statistical design and the analysis of gene expression microarray data. Genet Res 2001; 77:123-8.
29. Motakis E, Ivshina A V, Kuznetsov V A. Data-driven approach to predict survival of cancer patients: estimation of microarray genes' prediction significance by Cox proportional hazard regression model. IEEE Eng Med Biol Mag 2009; 28:58-66.
30. Kuznetsov V A S O, Miller L D, Ivshina A V. Statistically Weighted Voting Analysis of Microarrays for Molecular Pattern Selection and Discovery Cancer Genotypes. Intern J of Computer Sciences and Network Security 2006; 6:73-83.
31. McShane L M, Altman D G, Sauerbrei W, Taube S E, Gion M, Clark G M. REporting recommendations for tumour MARKer prognostic studies (REMARK). Br J Cancer 2005; 93:387-91.
32. Antonov A V, Knight R A, Melino G, Barley N A, Tsvetkov P O. MIRUMIR: an online tool to test microRNAs as biomarkers to predict survival in cancer using multiple clinical data sets. Cell Death Differ 2012.
33. Yang H, Kong W, He L, Zhao J J, O'Donnell J D, Wang J, Wenham R M, Coppola D, Kruk P A, Nicosia S V, Cheng J Q. MicroRNA expression profiling in human ovarian cancer: miR-214 induces cell survival and cisplatin resistance by targeting PTEN. Cancer Res 2008; 68:425-33.
34. Xu C X, Xu M, Tan L, Yang H, Permuth-Wey J, Kruk P A, Wenham R M, Nicosia S V, Lancaster J M, Sellers T A, Cheng J O. MicroRNA miR-214 regulates ovarian cancer cell sternness by targeting p53/Nanog. J Biol Chem 2012; 287:34970-8.
35. Xu D, Takeshita F, Hino Y, Fukunaga S, Kudo Y, Tamaki A, Matsunaga J, Takahashi R U, Takata T, Shimamoto A, Ochiya T, Tahara H. miR-22 represses cancer progression by inducing cellular senescence. J Cell Biol 2011; 193:409-24.
36. Ahmed N, Abubaker K, Findlay J, Quinn M. Epithelial mesenchymal transition and cancer stem cell-like phenotypes facilitate chemoresistance in recurrent ovarian cancer. Curr Cancer Drug Targets 2010; 10:268-78.
37. Marchini S, Fruscio R, Clivio L, Beltrame L, Porcu L, Nerini I F, Cavalieri D, Chiorino G, Cattoretti G, Mangioni C, Milani R, Torri V, et al. Resistance to platinum-based chemotherapy is associated with epithelial to mesenchymal transition in epithelial ovarian cancer. Eur J Cancer 2012.
38. Yang D, Sun Y, Hu L, Zheng H, Ji P, Pecot Chad V, Zhao Y, Reynolds S, Cheng H, Rupaimoole R, Cogdell D, Nykter M, et al. Integrated Analyses Identify a Master MicroRNA Regulatory Network for the Mesenchymal Subtype in Serous Ovarian Cancer. Cancer Cell 2013; 23:186-99.
39. Alvero A B, Chen R, Fu H H, Montagna M, Schwartz P E, Rutherford T, Silasi D A, Steffensen K D, Waldstrom M, Visintin I, Mor G. Molecular phenotyping of human ovarian cancer stem cells unravels the mechanisms for repair and chemoresistance. Cell Cycle 2009; 8:158-66.
40. Yin G, Chen R, Alvero A B, Fu H H, Holmberg J, Glackin C, Rutherford T, Mor G. TWISTing stemness, inflammation and proliferation of epithelial ovarian cancer cells through MI R199A2/214. Oncogene 2010; 29:3545-53.
41. Matei D, Emerson R E, Lai Y C, Baldridge L A, Rao J, Yiannoutsos C, Donner D D. Autocrine activation of PDGFRaIpha promotes the progression of ovarian cancer. Oncogene 2006; 25:2060-9.
42. Huber-Keener K J, Liu X, Wang Z, Wang Y, Freeman W, Wu S, Planas-Silva M D, Ren X, Cheng Y, Zhang Y, Vrana K, Liu C G, et al. Differential gene expression in tamoxifen-resistant breast cancer cells revealed by a new analytical model of RNA-Seq data. PLoS One 2012; 7:e41333.
43. Flahaut M, Meier R, Coulon A, Nardou K A, Niggli F K, Martinet D, Beckmann J S, Joseph J M, Muhlethaler-Mottet A, Gross N. The Wnt receptor FZD1 mediates chemoresistance in neuroblastoma through activation of the Wnt/beta-catenin pathway. Oncogene 2009; 28:2245-56.
44. Zhang H, Zhang X, Wu X, Li W, Su P, Cheng H, Xiang L, Gao P, Zhou G. Interference of Frizzled 1 (FZD1) reverses multidrug resistance in breast cancer cells through the Wnt/beta-catenin pathway. Cancer Lett 2012; 323:106-13.
45. Rosano L, Cianfrocca R, Spinella F, Di Castro V, Nicotra M R, Lucidi A, Ferrandina G, Natali P G, Bagnato A. Acquisition of chemoresistance and EMT phenotype is linked with activation of the endothelin A receptor pathway in ovarian carcinoma cells. Clin Cancer Res 2011; 17:2350-60.
46. Zhou H Y, Pon Y L, Wong A S. HGF/MET signaling in ovarian cancer. Curr Mol Med 2008; 8:469-80.
47. Gutova M, Najbauer J, Gevorgyan A, Metz M Z, Weng Y, Shih C C, Aboody K S. Identification of uPAR-positive chemoresistant cells in small cell lung cancer. PLoS One 2007; 2:e243.
48. Heileman J, Jansen M P, Span P N, van Staveren I L, Massuger L F, Meijer-van Gelder M E, Sweep F C, Ewing P C, van der Burg M E, Stoter G, Nooter K, Berns E M. Molecular profiling of platinum resistant ovarian cancer. Int J Cancer 2006; 118:1963-71.
49. Katsetos C D, Draber P. Tubulins as therapeutic targets in cancer: from bench to bedside. Current pharmaceutical design 2012; 18:2778-92.
50. De Donato M, Mariani M, Petrella L, Martinelli E, Zannoni G F, Vellone V, Ferrandina G, Shahabi S, Scambia G, Ferlini C. Class III beta-tubulin and the cytoskeletal gateway for drug resistance in ovarian cancer. Journal of cellular physiology 2012; 227:1034-41.
51. Barretina J, Caponigro G, Stransky N, Venkatesan K, Margolin A A, Kim S, Wilson C J, Lehar J, Kryukov G V, Sonkin D, Reddy A, Liu M, et al. The Cancer Cell Line Encyclopedia enables predictive modelling of anticancer drug sensitivity. Nature 2012; 483:603-7.
52. Heise C, Ganly I, Kim Y T, Sampson-Johannes A, Brown R, Kim D. Efficacy of a replication-selective adenovirus against ovarian carcinomatosis is dependent on tumor burden, viral replication and p53 status. Gene therapy 2000; 7:1925-9.
53. Behrens B C, Hamilton T C, Masuda H, Grotzinger K R, Whang-Peng J, Louie K G, Knutsen T, McKoy W M, Young R C, Ozols R F. Characterization of a cis-diamminedichloroplatinum(II)-resistant human ovarian cancer cell line and its use in evaluation of platinum analogues. Cancer Res 1987; 47:414-8.
54. Orlov Y L, Zhou J, Lipovich L, Shahab A, Kuznetsov V A. Quality assessment of the Affymetrix U133A&B probesets by target sequence mapping and expression data analysis. In Silico Biol 2007, 7(3):241-260.
55. Huang da W, Sherman B T, Lempicki R A: Systematic and integrative analysis of large gene lists using DAVID bioinformatics resources. Nat Protoc 2009, 4(1):44-57.
56. Kuznetsov V A, Ivshina A V, Sen'ko O V, Kuznetsova A V: Syndrome approach for computer recognition of fuzzy systems and its application to immunological diagnostics and prognosis of human cancer. Mathematical and Computer Modelling 1996, 23(6):95-119.
57. Agresti A: An Introduction to Categorical Data Analysis, 2nd Edition: Wiley; 2007

1-39. (canceled)

40. A method for the prognosis of overall survival or prediction of therapeutic outcome for a patient suffering from high-grade epithelial ovarian cancer (HG-EOC), comprising:
a. providing a sample from the patient,
b. determining the expression level of microRNA family member lethal-7b (let-7b) in the sample;
c. using the expression level of the let-7b to obtain the prognosis of overall survival or prediction of therapeutic outcome for the patient;
wherein the method comprises comparing the expression level of let-7b to an expression cutoff level of let-7b in HG-SOC patients in a comparison population, whereby a higher expression level of let-7b in the sample relative to the expression cutoff level is indicative of less favorable prognosis of overall survival or less favorable therapeutic outcome for the patient than the comparison population.

41. The method according to claim 40, wherein the cancer is high-grade serous epithelial ovarian cancer (HG-SOC).

42. The method according to claim 40, further comprising an operation of determining the expression level of at least one let-7 family member selected from the group consisting of let-7a, let-7c, let-7d, let-7e, let-7f, let-7g, let-7i, and miR-98 and further using the expression level of said at least one let-7 family member to obtain the prognosis of overall survival or prediction of therapeutic outcome for the patient.

43. The method according to claim 42, wherein the let-7a is selected from the group consisting of let-7a-1, let-7a-2, and let-7a-3.

44. The method according to claim 42, wherein the let-7f is selected from the group consisting of let-7f-1 and let-7f-2.

45. The method according to claim 40, further comprising the operation of determining the expression level of at least one microRNA associated with let-7b and/or at least one gene associated with let-7b and further using the expression level of the let-7b associated microRNA and/or let-7b associated gene to obtain the prognosis of an outcome or assessing the risk for the patient.

46. The method according to claim 45, wherein the expression level is compared to expression levels of the corresponding microRNA or gene in the HG-EOC patients in the comparison population to obtain the prognosis or risk assessment.

47. The method according to claim 45, wherein the microRNA is selected from the group consisting of miR-17-5p, miR-183, miR-96, miR-107, miR-106b, miR-25, miR-324-5p, miR-517c, miR-103, miR-362, miR-136, miR-320, and miR-486.

48. The method according to claim 45, wherein the gene is selected from the group consisting of DNMT1, CD93, ARPC1B, CD44, PIK3R1, GNG12, CCL2, PLAUR, LAMA4, VCL, FZD1, CALD1, EDNRA, TGFBR2, FGFR1, POLR2D, POLR2J, CDK4, CHEK1, CCT2, CDC6, TUBB, NCAPD2, NCAPG2, POLA2, TCP1, NCAPH, CBX3, and MIS12.

49. The method according to claim 46, wherein the expression level of let-7b, the expression level(s) of the microRNA(s) associated with let-7b and/or the expression level(s) of the gene(s) associated with let-7b stratify the comparison population into a plurality of subgroups with prognosis of different outcomes.

50. A method of treating high-grade epithelial ovarian cancer (HG-EOC) in a patient, the method comprising administering at least one agent capable of modulating the expression of let-7b and/or at least one gene associated with let-7b based on results of a method for the prognosis of overall survival or prediction of therapeutic outcome for a patient suffering from high-grade epithelial ovarian cancer (HG-EOC), comprising:
a. providing a sample from the patient,
b. determining the expression level of microRNA family member lethal-7b (let-7b) in the sample;
c. using the expression level of the let-7b to obtain the prognosis of overall survival or prediction of therapeutic outcome for the patient;
wherein the method comprises comparing the expression level of let-7b to an expression cutoff level of let-7b in HG-SOC patients in a comparison population, whereby a higher expression level of let-7b in the sample relative to the expression cutoff level is indicative of less favorable prognosis of overall survival or less favorable therapeutic outcome for the patient than the comparison population.

51. The method according to claim 50, wherein the gene is selected from the group consisting of DNMT1, CD93, ARPC1B, CD44, PIK3R1, GNG12, CCL2, PLAUR, LAMA4, VCL, FZD1, CALD1, EDNRA, TGFBR2, FGFR1, POLR2D, POLR2J, CDK4, CHEK1, CCT2, CDC6, TUBB, NCAPD2, NCAPG2, POLA2, TCP1, NCAPH, CBX3, and MIS12.

52. The method according to claim 50, wherein the agent is a polynucleotide and/or polypeptide capable of increasing or decreasing the expression of let-7b and/or the gene associated with let-7b.

53. A method for the prognosis of overall survival or prediction of therapeutic outcome for a patient suffering from high-grade epithelial ovarian cancer (HG-EOC), comprising:
a. providing a sample from the patient,
b. determining the expression level of at least one gene selected from the group consisting of DNMT1, CD93, ARPC1B, CD44, PIK3R1, GNG12, CCL2, PLAUR, LAMA4, VCL, FZD1, CALD1, EDNRA, TGFBR2, FGFR1, POLR2D, POLR2J, CDK4, CHEK1, CCT2, CDC6, TUBB, NCAPD2, NCAPG2, POLA2, TCP1, NCAPH, CBX3, and MIS12 in the sample;
c. using the expression level of the gene to obtain the prognosis of overall survival or prediction of therapeutic outcome for the patient.

54. A method for the prognosis of overall survival or prediction of therapeutic outcome for a patient suffering from high-grade epithelial ovarian cancer (HG-EOC), comprising:
a. providing a sample from the patient,
b. determining the expression level of genes PDGFRA, CAV2, FZD1, EDNRA, MMP13, HGF, PLAUR and COL3A1 in the sample; and
c. using the expression level of the genes to obtain the prognosis of overall survival or prediction of therapeutic outcome for the patient.

55. The method according to claim 54, wherein the expression level of the or each gene is compared to expression levels of the one or more genes in HG-EOC patients in a comparison population to obtain the prognosis of overall survival or prediction of therapeutic outcome.

56. The method according to claim 55, comprising providing threshold data which, for each gene, represent one or more expression level thresholds, the expression level thresholds stratifying the comparison population into a plurality of subgroups; and comparing the expression level of the one or more genes in the patient to the one or more expression level thresholds for respective genes to classify the patient into one of the subgroups, to thereby obtain the prognosis of overall survival or prediction of therapeutic outcome.

57. The method according to claim 56, wherein a prognosis or prediction is determined for each one of a plurality of the group of genes, and further comprising generating a consensus prognosis or prediction from the individual prognoses or predictions.

58. A method for the prognosis of overall survival or prediction of therapeutic outcome for a patient suffering from high-grade epithelial ovarian cancer (HG-EOC), comprising:
a. providing a sample from the patient,
b. determining the expression level of at least one microRNA selected from the group consisting of miR-17-5p, miR-183, miR-96, miR-107, miR-106b, miR-25, miR-324-5p, miR-517c, miR-103, miR-362, miR-136, miR-320, and miR-486 in the sample;
c. using the expression level of the microRNA to obtain the prognosis of overall survival or prediction of therapeutic outcome.

59. The method according to claim 58, wherein the expression level of the one or more microRNAs is compared to expression levels of the one or more microRNAs in HG-EOC patients in a comparison population to obtain the prognosis of overall survival or prediction of therapeutic outcome.

60. The method according to claim 59, comprising providing threshold data which, for each microRNA, represent one or more expression level thresholds, the expression level thresholds stratifying the comparison population into a plurality of subgroups; and comparing the expression level of the one or more microRNAs in the patient to the one or more expression level thresholds for respective microRNAs to classify the patient into one of the subgroups, to thereby obtain the prognosis of overall survival or prediction of therapeutic outcome.

61. The method according to claim 60, wherein a prognosis or prediction is determined for each one of a plurality of the group of microRNAs, and further comprising generating a consensus prognosis or prediction from the individual prognoses or predictions.
